(19) United States
(12) Patent Application Publication
(10) Pub. No.: US 2023/0122229 A1
(43) Pub. Date: Apr. 20, 2023

(54) USE OF PULSED FOCUSED ULTRASOUND THERAPY IN COMBINATION WITH MESENCHYMAL STROMAL CELLS OR MESENCHYMAL STROMAL CELL-DERIVED EXTRACELLULAR VESICLES FOR REGENERATION OF KIDNEY TISSUE

(71) Applicant: The Board o fTrustees of the Leland Stanford Junior University, Stanford, CA (US)

(72) Inventors: Avnesh S. Thakor, Palo Alto, CA (US); Mujib Ullah, Redwood City, CA (US)

(21) Appl. No.: 17/905,402

(22) PCT Filed: Mar. 4, 2021
(86) PCT No.: PCT/US2021/020835

§ 371 Date: Aug. 31, 2022

Related U.S. Application Data
(60) Provisional application No. 62/986,435, filed on Mar. 6, 2020.

(51) Int. Cl. A61N 7/00; A61K 35/28; C12N 5/0775; C12Q 1/6809

(52) U.S. Cl. CPC A61N 7/00; A61K 35/28; C12N 5/0663; C12Q 1/6809; A61B 2090/374

(57) ABSTRACT

Methods for treating kidney damage are provided. The methods utilize a combination of therapies, including the administration of pulsed focused ultrasound (pFUS) therapy with mesenchymal stromal cells (MSCs) and/or MSC-derived extracellular vesicles. Additionally, methods are provided for screening candidate therapeutic agents for treating kidney damage that have the ability to increase expression of HSP20 or HSP40, decrease expression of HSP70 or HSP90, enhance activation of PI3K/Akt signaling, or suppress the NLRP3 inflammasome and inflammasome-mediated inflammation.

### BACKGROUND OF THE INVENTION

Acute kidney injury (AKI) is characterized by a sudden decline in renal function. It is a common complication of conditions like chronic hypertension, heart failure, and kidney ischemia, and frequently affects hospitalized patients following surgical procedures or administration of nephrotoxic drugs (Levey et al. (2017) Ann Intern Med 167, ITC66-ITC80). In recent years, there has been a substantial increase in hospitalizations for AKI. The Centers for Disease Control and Prevention (CDC) estimates that in the United States, AKI hospitalizations increased from around 1 million in 2000 to nearly 4 million in 2014 (Pavkov et al. (2018) MMWR Morb Mortal Wkly Rep 67, 289-293). Similar trends have been found in other countries, including a 3-fold increase in Denmark (Carlson et al. (2016) PLoS One 11, e0159944) and a 13-fold increase in England (Kolhe et al. (2015) Kidney Int 88, 1161-1169) during similar time frames. AKI is an independent risk factor for end-stage renal disease (ESRD) and death (Coca et al. (2012) Kidney Int 81, 442-448). Moreover, multiple studies have also shown that AKI can trigger the onset of chronic kidney disease (CKD), or exacerbate it when already present (Coca et al. (2012), supra; Chawla et al. (2012) Kidney Int 82, 516-524; Chawla et al. (2014) N Engl J Med 371, 58-66; Pannu et al. (2013) Curr Opin Nephrol Hypertens 22, 351-356; Coca et al. (2011) Nephron Clin Pract 119 Suppl 1, c19-24; Hsu et al. (2016) Semin Nephrol 36, 283-292). Given that there are currently no approved therapies for reversing kidney damage, the standard approach to AKI is mostly supportive in nature: maintaining blood volume through intravenous (IV) fluids or diuretics, balancing electrolyte concentrations, and initiating dialysis if necessary. However, even with supportive treatment, AKI is associated with significantly increased morbidity and mortality, with substantially increased costs and hospital stay lengths (Chertow et al. (2005) J Am Soc Nephrol 16, 3365-3370; Leither et al. (2019) Nephrol Dial Transplant 34, 493-501). Thus, there is an unmet need for therapies that can promote the repair and regeneration of injured kidneys and prevent the progression to worsening kidney disease.

Stem cell therapy is a promising approach in regenerative medicine that has been shown to have significant potential for the repair of damaged kidney tissue. Whereas traditional pharmaceutical approaches target only one aspect of the complex pathophysiology of AKI, it has been proposed that stem cells might work through multiple mechanisms to effect tissue repair (Liu et al. (2008) Crit Care Med 36, S187-192). Mesenchymal stromal cells (MSCs) have been one of the most popular platforms for stem cell therapy. MSCs are multipotent cells with potent angiogenic and immunomodulatory properties (Bruno et al. (2014) Stem Cells Transl Med 3, 1451-1455). In animal studies, it has been shown that intravenously (IV) infused bone marrow-derived MSCs (BM- MSCs) home to the kidney and stimulate repair and regeneration following AKI (Herrera et al. (2004) Int J Mol Med 14, 1035-1041; Morigi et al. (2014) Nephron Exp Nephrol 126, 59). These MSCs exert their therapeutic effects not by differentiating into new tissue, but rather by acting as a mobile reservoir of regenerative molecules (Caplan et al. (2009) J Pathol 217, 318-324). These soluble factors are able to stimulate regeneration through pro-survival, mitogenic, anti-inflammatory, and angiogenic effects, which not only protect surviving kidney cells, but also promote their proliferation (Morigi et al. (2014), supra). Although MSC therapy for AKI has shown promising effects in preclinical settings, many hurdles still limit its efficacy, the most pressing of which is the inefficiency of MSC homing. Since such therapies appear to depend on the paracrine action of MSCs, the proximity of infused MSCs to damaged tissue is thus essential. However, many studies have shown that IV infused BM-MSCs are largely trapped in the lung microvasculature, in what is known as the pulmonary first-pass effect (Schrepfer et al. (2007) Transplant Proc 39, 573-576; Santeramo et al. (2017) Stem Cells Transl Med 6, 1373-1384; Leibacher et al. (2016) Stem Cell Res Ther 7, 7; Eggenhofer et al. (2012) Front Immunol 3, 297; Fischer et al. (2009) Stem Cells Dev 18, 683-692).

Thus, there remains a need for better methods of treating AKI and regenerating and repairing kidney tissue.

### SUMMARY OF THE INVENTION

Safe and efficacious methods for treating kidney damage are provided. The methods utilize a combination of therapies, including the administration of pulsed focused ultrasound (pFUS) therapy with mesenchymal stromal cells (MSCs) and/or MSC-derived extracellular vesicles (e.g., exosomes or microvesicles). Pulsed focused ultrasound is a non-invasive technology that utilizes sound waves that penetrate through the body and, in the methods described herein, is used to target the kidney to alter the microenvironment to facilitate tissue regeneration either directly (i.e., affect the intrinsic regenerative and protective capacity of the tissue) or indirectly (e.g., homing of MSCs). Additionally, methods are provided for screening candidate therapeutic agents for treating kidney damage that have the ability to increase expression of HSP20 or HSP40, decrease expression of HSP70 or HSP90, enhance activation of PI3K/Akt signaling, or suppress the NLRP3 inflammasome and inflammasome-mediated inflammation.

In one aspect, a method of treating damaged kidney tissue in a subject is provided, the method comprising locally administering to the damaged kidney tissue a therapeutically effective amount of pulsed focused ultrasound (pFUS) therapy in combination with a therapeutically effective amount of MSCs or MSC-derived extracellular vesicles.

In certain embodiments, the MSCs or the MSC-derived extracellular vesicles are administered locally to the damaged kidney tissue intra-arterially via a renal artery.

In certain embodiments, the pFUS is administered with an ultrasound frequency ranging from about 20 kHz to about 5.0 MHz, about 0.7 MHz to about 3.0 MHz, or about 1.0 MHz to about 1.1 MHz, including any ultrasound frequency within these ranges, such as 0.2, 0.4, 0.6, 0.8, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 3.0, 3.2, 3.4, 3.6, 3.8, 4.0, 4.2, 4.4, 4.6, 4.8, or 5.0 MHz.

In certain embodiments, the pFUS is administered with a pulse repetition frequency (PRF) ranging from 0.1 Hz to 1000 Hz, 1 Hz to 100 Hz, or about 5 Hz to 20 Hz, or any PRF with these ranges, such as 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, or 1000 Hz. In certain embodiments, the pFUS is administered with a PRF of about 5 Hz.

In certain embodiments, the pFUS is administered with an ultrasound duty cycle ranging from 0.01% to 100% or 1% to 20%, including any ultrasound duty cycle within these ranges such as 0.01%, 0.1 %, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 90%, 95%, or 100%. In some embodiments, the pFUS is administered with an ultrasound duty cycle of about 5%. In some embodiments, the pFUS therapy is administered with an ultrasound duty cycle of less than 1%.

In certain embodiments, the pFUS is administered with a negative peak pressure (NPP) ranging from 0.1 MPa to 10 MPa, including any NPP within this range such as 0.1, 0.2, 0.3, 0.4, 0.5, 0.6, 0.7, 0.8, 0.9, 1, 2, 3, 4, 5, 6, 7, 8, 9, or 10 MPa. In some embodiments, the pFUS is administered with a negative peak pressure (NPP) of up to 3 MPa. In some embodiments, the pFUS is administered with a negative peak pressure (NPP) of up to 3 MPa. In some embodiments, the NPP is about 2.9 MPa.

In certain embodiments, the pFUS is administered to the subject for a time ranging from about 20 seconds to about 7 minutes, including any amount of time within this range, such as 20 seconds, 25 seconds, 30 seconds, 35 seconds, 40 seconds, 45 seconds, 50 seconds, 55 seconds, 1 minute, 1.25 minutes, 1.5 minutes, 1.75 minutes, 2 minutes, 2.25 minutes, 2.5 minutes, 2.75 minutes, 3 minutes, 3.25 minutes, 3.5 minutes, 3.75 minutes, 4 minutes, 4.25 minutes, 4.5 minutes, 4.75 minutes, 5 minutes, 5.25 minutes,, 5.5 minutes, 5.75 minutes, 6 minutes, 6.25 minutes, 6.5 minutes, 6.75 minutes, or 7 minutes. In some embodiments, the pFUS therapy is administered to the subject for at least 20 seconds. In some embodiments, the pFUS therapy is administered to the subject for a period ranging from about 1 minute to about 5 minutes. In one embodiment, the pFUS therapy is administered to the subject for about 160 seconds.

In certain embodiments, the pFUS is administered with a spatial average pulse average intensity (Iₛₐₚₐ) of about 272 W/cm².

In certain embodiments, the pFUS therapy is administered at a single location or at multiple locations in the kidney. In some embodiments, some or all of the multiple locations are overlapping.

In certain embodiments, the method further comprises imaging the damaged kidney tissue, for example, by ultrasound or a non-ultrasound imaging method such as, but not limited to, magnetic resonance imaging (MRI), computed tomography (CT), or scintigraphy.

In certain embodiments, the MSCs are from bone marrow or adipose tissue.

In certain embodiments, the kidney tissue is damaged from an acute kidney injury (e.g., such as caused by chemotherapy, a chemical exposure, surgery, or a traumatic physical injury) or chronic kidney disease (e.g., such as caused by high blood pressure, diabetes, glomerulonephritis, or polycystic kidney disease).

In certain embodiments, the MSC-derived extracellular vesicles are exosomes or microvesicles. In some embodiments, the MSC-derived exosomes comprise one or more surface markers selected from the group consisting of CD9, CD63, and TSG101. In some embodiments, the MSC-derived exosomes comprise the surface markers: CD9, CD63, and TSG101.

In certain embodiments, the MSC-derived extracellular vesicles have diameters ranging from about 20 nm to 180 nm, including any size within this range such as 20, 30 40, 50, 60, 70, 80, 90, 100, 110, 120, 130, 140, 150, 160, 170, or 180 nm. In some embodiments, the MSC-derived extracellular vesicles have a mean diameter of 118 nm.

In certain embodiments, multiple cycles of treatment are administered to the subject.

In certain embodiments, the treatment suppresses NLRP3 inflammasome-mediated inflammation.

In another aspect, a method of suppressing NLRP3 inflammasome-mediated inflammation in a subject is provided, the method comprising locally administering to damaged kidney tissue an effective amount of pulsed focused ultrasound (pFUS) in combination with an effective amount of mesenchymal stromal cells (MSCs) or MSC-derived extracellular vesicles.

In another aspect, a method of screening a candidate agent for treating kidney damage is provided, the method comprising: a) contacting a test population of kidney cells or kidney tissue from a damaged kidney with the candidate agent; and b) measuring expression of HSP20, HSP40, HSP70, or HSP90, PI3K/Akt signaling, or NLRP3 inflammasome activity in the test population of kidney cells or kidney tissue, wherein increased expression of HSP20 or HSP40, decreased expression of HSP70 or HSP90, increased activation of PI3K/Akt signaling, or decreased NLRP3 inflammasome activity in the test population of kidney cells or kidney tissue compared to that in a negative control population of kidney cells or kidney tissue that are not contacted with the candidate agent indicates that the candidate agent may be useful for treating kidney damage.

In certain embodiments, the test population of kidney cells or kidney tissue is obtained from a kidney damaged by an acute kidney injury or chronic kidney disease.

Candidate agents may include, without limitation, small molecules, peptides, proteins, aptamers, antibodies, antibody mimetics, transcription factors, hormones, nucleic acids, or clustered regularly interspaced short palindromic repeats (CRISPR) systems that increase HSP20 or HSP40 biological activity or expression, decrease HSP70 or HSP90 biological activity or expression, activate PI3K/Akt signaling, or decrease NLRP3 inflammasome biological activity or inflammasome-mediated inflammatory responses.

In certain embodiments, the candidate agent is a CRISPR system that targets a HSP20 gene, a HSP40 gene, a HSP70 gene, or a HSP90 gene; or a HSP20 RNA transcript, a HSP40 RNA transcript, a HSP70 RNA transcript, or a HSP90 RNA transcript; or makes epigenetic changes that increase expression of HSP20 or HSP40 or decrease expression of HSP70 or HSP90. In some embodiments, the CRISPR system comprises Cas9, Cas12a, Cas12d, Cas13a, Cas13b, Cas13d, or a dead Cas9 (dCas9).

In some embodiments, the candidate agent is an antibody, wherein the antibody is selected from the group consisting of a polyclonal antibody, a monoclonal antibody, a chimeric antibody, a humanized antibody, a F(ab) fragment, a F(ab')2 fragment, a Fᵥ fragment, and a nanobody.

In another aspect, a kidney therapeutic agent identified by the screening methods described herein is provided. In certain embodiments, the kidney therapeutic agent is provided in a pharmaceutical formulation suitable for administration to a patient. Formulations of interest include, without limitation, formulations for local or systemic administration, including oral or parenteral administration. In some embodiments, the composition comprises a pharmaceutically acceptable excipient. In some embodiments, the composition further comprises a pharmaceutically acceptable carrier including, without limitation, a cream, emulsion, gel, liposome, nanoparticle, or ointment.

Such agents identified by screening, as described herein, may be useful in treating a damaged kidney, including, without limitation, damage caused by an acute kidney injury or chronic kidney disease.

### BRIEF DESCRIPTION OF THE DRAWINGS

FIGS. 1A-1C. Physical parameters and biochemistry of AKI mice following cisplatin injection. (FIG. 1A) Animal body weight. (FIG. 1B) Plasma creatinine levels in animals. (FIG. 1C) Blood urea nitrogen levels in animals. Each group has n=6 except in AKI where n=5. Significant difference ^{a}P < 0.05: relative to untreated control; ^{b}P < 0.05: relative to AKI; ^{c}P < 0.05: relative to AKI+BM-MSCs; ^{d}P < 0.05: relative to AKI+BM-MSCs+pFUS; *P < 0.05: all time points relative to day 0.

FIGS. 2A-2D. Homing of BM-MSCs. (FIG. 2A) Homing of BM-MSCs, shown by green color due to expression of GFP in kidney sections. Each group has n=6 except AKI where n=5 mice, 20 images per animal were taken. (FIG. 2B) Quantification of GFP signal by flow cytometry in kidney homogenized cells. Each group has n=6 except in AKI where n=5. (FIG. 2C) PCR showing the presence of human GAPDH in treated kidneys. Each group has n=5 mice. (FIG. 2D) Cytokine expression within kidney lysates samples of kidneys treated with pFUS, measured 2 days post-sonication. Each group has n=2 mice. Scale bar represents 100 µm. Significant difference ^{a}P < 0.05: relative to untreated control; ^{b}P < 0.05: relative to AKI; ^{c}P < 0.05: relative to AKI+BM-MSCs; ^{d}P < 0.05: relative to AKI+BM-MSCs+pFUS.

FIGS. 3A-3B. Histological analysis of kidneys from untreated and treated AKI mice. (FIG. 3A) Hematoxylin and eosin staining showing the different level of injury, cast formation and fibrosis across different experimental groups. Scale bar represents 100 µm. Each group has n=6 except AKI where n=5, average of 20 images per animal were taken. (FIG. 3B) Quantification of histological data. Each group has n=6 except in AKI where n=5. Significant difference ^{a}P < 0.05: relative to untreated control; ^{b}P < 0.05: relative to AKI; ^{c}P < 0.05: relative to AKI+BM-MSCs; ^{d}P < 0.05: relative to AKI+BM-MSCs+pFUS.

FIGS. 4A-4D. Immunohistochemistry and assessment of inflammation in kidneys from untreated and treated AKI mice. (FIG. 4A) Immunohistochemical staining of inflammatory markers: TNFα, IL-6, and MCP-1 in AKI mice which are untreated or treated with BM-MSCs alone or BM-MSCs + pFUS. (FIG. 4B) Serum cytokine measurement of inflammatory markers: TNFα, IL-6, and MCP-1 in AKI mice which are untreated or treated with BM-MSCs alone or BM-MSCs + pFUS. (FIG. 4C) qPCR, gene expression of inflammatory markers: TNFα, IL-6, and IGF-1 in AKI mice which are untreated or treated with BM-MSCs alone or BM-MSCs + pFUS. (FIG. 4D) Relative Lactate dehydrogenase (LDH) expression compared to isotype control measured by flow cytometry in AKI mice which are untreated or treated with BM-MSCs alone or BM-MSCs + pFUS. Scale bar represents 100 µm. Each group has n=6 except in AKI where n=5. Significant difference ^{a}P < 0.05: relative to untreated control; ^{b}P < 0.05: relative to AKI; ^{c}P < 0.05: relative to AKI+BM-MSCs; ^{d}P < 0.05: relative to AKI+BM-MSCs+pFUS.

FIGS. 5A-5D. Apoptosis, cell death and protein arrays in kidneys from untreated and treated AKI mice. (FIG. 5A) Western blot showing the expression of Bax, PARP, Bcl2, caspase-3, and beta actin. (FIG. 5B) qPCR showing the expression of Bax, PARP, Bcl2, and caspase-3, normalized to GAPDH. (FIG. 5C) Western blot showing the expression of p-ERK1/2, p-AKT, and p-AMPK (targets of HSP20). (FIG. 5D) Quantification of western blot showing the expression of p-ERK1/2, p-AKT, and p-AMPK (targets of HSP20). Each group has n=6 except in AKI where n=5. Significant difference ^{a}P < 0.05: relative to untreated control; ^{b}P < 0.05: relative to AKI; ^{c}P < 0.05: relative to AKI+BM-MSCs; ^{d}P < 0.05: relative to AKI+BM-MSCs+pFUS.

FIGS. 6A-6D. Protein arrays to identify the mechanistic targets in kidneys from untreated and treated AKI mice. (FIG. 6A) Quantification of protein arrays showing the comparative expression of various heat shock proteins taken from the protein array data. (FIG. 6B) Blot of protein arrays showing the expression of various heat shock proteins in the form of expression dots in AKI mice group compared to treatment with BM-MSCs alone or BM-MSCs + pFUS. (FIGS. 6C, 6D) Validation and Quantification of heat shock protein by western blot, showing the expression of HSP-20 in AKI mice group compared to treatment with BM-MSCs alone or BM-MSCs + pFUS. Each group has n=6 except in AKI where n=5. Significant difference ^{a}P < 0.05: relative to untreated control; ^{b}P < 0.05: relative to AKI; ^{c}P < 0.05: relative to AKI+BM-MSCs; ^{d}P < 0.05: relative to AKI+BM-MSCs+pFUS.

FIGS. 7A-7D. Validation of HSP20 and its targets in cultured human embryonic kidney cells (HEK293) and HEK293- SiRNA HSP20-knockdown cells. (FIG. 7A) Western blot of HSP20 and HSP40 in cultured normal HEK293 cells, and HEK293 siRNA HSP20-knockdown cells. (FIG. 7B) Western blot of p-AKT in cultured HEK293 cells and HEK293 siRNA HSP20-knockdown cells. (FIG. 7C) qRT-PCR of HSP20, HSP40, and p-AKT in HEK293 cells and HEK293 siRNA HSP20-knockdown cells. (FIG. 7D) Beta galactosidase staining, blue color showing cell senescence in cultured normal HEK293 cells, and HEK293 siRNA HSP20-knockdown cells. Significant difference ^{a}P < 0.05: HEK293 vs. HEK293-SiRNA cells.

FIGS. 8A-8C. Effects of p-FUS on HEK293 cells. (FIG. 8A) Western blot of HSP20 and HSP40 in cultured HEK293 cells, untreated or induced with pFUS. (FIG. 8B) RT-PCR of HSP20 in cultured HEK293 cells, untreated or induced with pFUS. (FIG. 8C) Western blot of p-AKT in cultured HEK293 cells, untreated or induced with pFUS.

FIGS. 9A-9G. Characterization of BM-MSCs. (FIG. 9A) Study protocol. (FIG. 9B) Morphology of BM-MSCs. (FIG. 9C) Surface markers to validate identity of BM-MSCs. (FIG. 9D) Growth curve of BM-MSCs. (FIG. 9E) Colony formation assay for BM-MSCs. (FIG. 9F) Population doubling time for BM-MSCs. Scale bar represents 200 µm.

FIGS. 10A-10F. Selective ligation allows for selective kidney labeling. (FIG. 10A) Normal anatomy of the mouse abdominal aorta, showing origin of the celiac trunk (CT), superior mesenteric artery (SMA), renal arteries, and kidneys. (FIG. 10B) Suture ligation sites, including the proximal aorta between the origins of the CT and SMA, the SMA, and the distal aorta. A metal clip is placed temporarily on the left renal artery to allow delivery of therapeutics first to the right renal artery. (FIG. 10C) Selective ligation and clip placement shown inside the mouse abdomen. (FIG. 10D) Result of tattoo dye injection into the distal aorta without described ligation technique. (FIGS. 10E, 10F) Result of tattoo dye injection with our ligation and clipping technique.

FIGS. 11A-11F. Physiological and molecular markers of kidney injury. (FIG. 11A) Study protocol. (FIG. 11B) Gross appearance of kidney. (FIG. 11C) Percent survival, animal body weight, and kidney weight. (FIG. 11D) Serum concentration of blood urea nitrogen (BUN), creatinine, and NGAL, as measured by serum ELISA. (FIG. 11E) mRNA expression of KIM-1, TIMP-1, and NGAL in kidney lysates, as measured by qRT-PCR. (FIG. 11F) Urine concentrations of KIM-1, TIMP-1, and NGAL, as measured by urine ELISA. Measurements were taken at day 12. Each group has n = 8 mice, except for survival data for which n = 10 for untreated controls and n = 20 mice for other groups. Significant difference ^{a}P < 0.05: relative to untreated control group; ^{b}P < 0.05: relative to AKI+IA saline group.

FIGS. 12A-12D. Inflammatory cytokines (FIG. 12A) Hematoxylin & eosin (H&E) staining and immunohistochemistry (IHC) of kidney tissue, stained for TNF-α and NF-_{K}B, and quantification of tubular casts. (FIG. 12B) Serum concentrations of TNF-α and IL-6, as measured by serum ELISA. (FIG. 12C) Western blot and quantification of NF-_{K}B and β-actin from kidney lysate. (FIG. 12D) mRNA expression of NF-_{K}B, as measured by qRT-PCR. Measurements were taken at day 12. Each group has n = 5 mice for TNF-α and IL-6 measurements and n = 3 mice for NF-_{K}B measurements. Significant difference ^{a}P < 0.05: relative to untreated control group; ^{b}P < 0.05: relative to AKI+IA saline group.

FIGS. 13A-13C. Proliferation and regeneration markers (FIG. 13A) Immunohistochemistry (IHC) of FGF2 and Ki67 in kidney tissue, and quantification of Ki67+ cells. (FIG. 13B) Western blot and quantification of Ki67, FGF2, FGF23, and β-actin from kidney lysate. (FIG. 13C) Western blot and quantification of pAMPK, pERK, and β-actin from kidney lysate. Measurements were taken at day 12. Each group has n= 3 pooled mice. Significant difference ^{a}P < 0.05: relative to untreated control group; ^{b}P < 0.05: relative to AKI+IA saline group.

FIGS. 14A-14B. Exosome Characterization (FIG. 14A) Transmission electron microscopy (TEM) of exosomes, and distribution of exosome size measured by nanoparticle tracking analysis. (FIG. 14B) Validation of exosome surface markers CD9, CD63, and TSG101 by Western blot.

FIGS. 15A-15D. Physiological and biochemical parameters. FIG. 15A. Characterization of EVs by transmission electron microscopy (left), size distribution measured by Nanosight tracking analysis (middle), and Western blot confirmation of EV markers CD9, CD63, and TSG101, normalized to CD81. Scale bar 200 nm. FIG. 15B. Study protocol and treatment plan. FIG. 15C. Survival rate at day 12 and kidney weight after cisplatin injection. FIG. 15D. ELISA for blood urea nitrogen, creatinine, and NGAL level measured in blood plasma. Each group has n = 6 mice except for survival data, where n = 10. Significant difference ap < 0.05: relative to untreated control; ^{b}p < 0.05: relative to AKI; ^{c}p < 0.05: relative to AKI-EV; ^{d}p < 0.05: relative to AKI-EV-pFUS.

FIGS. 16A-16B. EV homing and kidney histology. FIG. 16A. Homing of GFP-labeled EVs to the kidney, as measured by immunofluorescence (left) and flow cytometry in homogenized kidney samples (right). Scale bar represents 50 µm. FIG. 16B. Hematoxylin and eosin staining of kidney sections, showing various levels of injury and cast formation (left), and quantification of injury score (right). Scale bar represents 100 µm. Each group has n = 6 mice. Significant difference ^{a}p < 0.05: relative to untreated control; ^{b}p < 0.05: relative to AKI; ^{c}p < 0.05: relative to AKI-EV; ^{d}p < 0.05: relative to AKI-EV-pFUS.

FIGS. 17A-17D. Molecular markers of kidney injury. FIG. 17A. Immunohistochemical staining showing expression of injury markers KIM-1 and TIMP-1. Scale bar represents 100 µm. FIG. 17B. Western blot for KIM-1, NGAL, TIMP-1, and β-actin (left), and their respective quantification (right). FIG. 17C. Quantitative real-time PCR for Kim1, Ngal, and Timp1 in the kidney tissue. FIG. 17D. ELISA for KIM-1, NGAL, and TIMP-1 measured in the urine. Each group has n = 5 mice. Significant difference ^{a}p < 0.05: relative to untreated control; ^{b}p < 0.05: relative to AKI; ^{c}p < 0.05: relative to AKI-EV; ^{d}p < 0.05: relative to AKI-EV-pFUS.

FIGS. 18A-18D. Inflammatory cytokines. FIG. 18A. Immunohistochemistry staining for inflammatory markers TNE-α and IL-6 in kidney tissue. FIG. 18B. Western blot on kidney tissue measuring inflammatory markers TNE-α and NE-KB (left), alongside their quantification (right). FIG. 18C. Quantitative real-time PCR on kidney tissue measuring inflammatory markers Nfkb, IL6, and TNFα. FIG. 18D. ELISA measurement of blood serum concentrations of cytokines IL-1β, IL-6, and TNF-α. Each group has n = 5 mice. Significant difference ^{a}p < 0.05: relative to untreated control; ^{b}p < 0.05: relative to AKI; ^{c}p < 0.05: relative to AKI-EV; dp < 0.05: relative to AKI-EV-pFUS.

FIGS. 19A-19D. Proliferation and apoptosis. FIG. 19A. Immunohistochemical staining for proliferation marker Ki67 in kidney tissue (left), alongside quantification of the percentage of Ki67-positive cells (right). FIG. 19B. Western blot on kidney tissue measuring proliferation markers PCNA, VEGF, survivin, and β-actin (left) alongside quantification (right). FIG. 19C. Immunohistochemical staining for apoptosis marker Caspase-3 in kidney tissue. FIG. 19D. Western blot on kidney tissue measuring apoptosis markers BAX, Bcl-2, and Caspase-3 (left) alongside quantification (right). Each group has n = 3 mice. Significant difference ^{a}p < 0.05: relative to untreated control; ^{b}p < 0.05: relative to AKI; ^{c}p < 0.05: relative to AKI-EV; ^{d}p < 0.05: relative to AKI-EV-pFUS.

FIGS. 20A-20C. Signaling pathway analysis. FIG. 20A. Western blot and protein quantification showing the expression of p-ERK, ERK, p-MEK, MEK, and β-actin in kidney tissue. FIG. 20B. Western blot and protein quantification showing the expression of p-Akt, Akt, and β-actin in kidney tissue. FIG. 20C. Western blot and protein quantification showing the expression of SIRT3, eNOS, and β-actin in kidney tissue. Each group has n = 3 mice. Significant difference ^{a}p < 0.05: relative to untreated control; ^{b}p < 0.05: relative to AKI; ^{c}p < 0.05: relative to AKI-EV; ^{d}p < 0.05: relative to AKI-EV-pFUS.

FIGS. 21A-21C. Physiological and biochemical measures of kidney function. (FIG. 21A) Animal body weight and kidney weight. (FIG. 21B) ELISA of blood urea nitrogen (BUN) and serum creatinine. (FIG. 21C) ELISA of molecular kidney injury markers KIM-1 and NGAL. Measurements were taken 9 days following cisplatin treatment. Each group has n = 5 mice. Significant difference ^{a} p < 0.05 relative to untreated control; ^{b} p < 0.05 relative to AKI; ^{c} p < 0.05 relative to AKI + EVs; ^{d} p < 0.05 relative to AKI + EVs + pFUS.

FIGS. 22A-22D. HSP70 regulation of the NLRP3 inflammasome. (FIG. 22A) Western blot and quantification showing the expression of HSP70, HSP90, NLRP3, and β-actin in kidney tissue. (FIG. 22B) Immunohistochemical staining for NLRP3 in kidney tissue. (FIG. 22C) qRT-PCR measurement of inflammasome components NLRP3, ASC, and Caspase-1 expression in kidney tissue, normalized to GAPDH. (FIG. 22D) Western blot and protein quantification showing expression of HSP70, HSP90, NLRP3, and β-actin in human embryonic epithelial (HEK) cells treated with DMSO (control), an HSP70-siRNA, or 7BIO. Each group has n = 3 mice. Significant difference ^{a} p < 0.05 relative to untreated control; ^{b} p < 0.05 relative to AKI; ^{c} p < 0.05 relative to AKI + EVs; ^{d}p < 0.05 relative to AKI + EVs + pFUS; * p < 0.05 relative to DMSO control; ^{#} p < 0.05 relative to HSP70-siRNA.

FIGS. 23A-23C. Inflammatory cytokines. (FIG. 23A) Immunohistochemical staining for IL-1 β and IL-18 on kidney sections. (FIG. 23B) Western blot and quantification of IL-1 β and IL-18, n = 3 for each group. (FIG. 23C) ELISA quantification of serum NLRP3, IL-6 and TNF-α, n = 5 for each group. Significant difference ^{a} p < 0.05 relative to untreated control; ^{b} p < 0.05 relative to AKI; ^{c} p < 0.05 relative to AKI + EVs; ^{d} p < 0.05 relative to AKI + EVs + pFUS.

FIG. 24. Proposed mechanism of NLRP3 inflammasome suppression. Schematic showing the proposed mechanism by which pFUS and EVs suppress HSP70, the latter of which acts as a positive regulator of the NLRP3 inflammasome.

### DETAILED DESCRIPTION

Methods for treating kidney damage are provided. The methods utilize a combination of therapies, including the administration of pulsed focused ultrasound (pFUS) therapy with mesenchymal stromal cells (MSCs) and/or MSC-derived exosomes. Additionally, methods are provided for screening candidate therapeutic agents for treating kidney damage that have the ability to increase expression of HSP20 or HSP40 or enhance activation of PI3K/Akt signaling.

Before the treatment and screening methods are further described, it is to be understood that this invention is not limited to a particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited.

It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As used herein the singular forms “a”, “an”, and “the” include plural referents unless the context clearly dictates otherwise. Thus, for example, reference to “a cell” includes a plurality of such cells and reference to “the activator” includes reference to one or more activators and equivalents thereof, e.g., agonists, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The term “about,” particularly in reference to a given quantity, is meant to encompass deviations of plus or minus five percent.

As used herein, the terms “mesenchymal stromal cells” and “mesenchymal stem cells” are used interchangeably and refer to multipotent cells derived from connective tissue. The terms encompass MSCs derived from various sources including, without limitation, bone marrow, adipose tissue, umbilical cord tissue, molar tooth bud tissue, and amniotic fluid.

The term “administering” is intended to include routes of administration which allow a therapeutic agent or combination therapy with pFUS and MSCs and/or MSC-derived extracellular vesicles to perform the intended function of promoting regeneration and/or restoring function of damaged kidney tissue. Additionally, administering a treatment, as described herein, may reduce levels of inflammatory cytokines, decrease apoptosis, increase cell survival and proliferation in kidney tissue, upregulate expression of heat shock proteins such as HSP20 and HSP40, downregulate expression of heat shock proteins such as HSP70 and HSP90, promote activation of PI3K/Akt signaling or homing of MSCs to damaged kidney tissue, or suppress NLRP3 inflammasome activity and inflammasome-mediated inflammation. Examples of routes of administration which can be used include local administration to damaged kidney tissue, injection (e.g., intravenous, intra-arterial (e.g., via renal artery), subcutaneous, parenteral, etc.), or surgical transplantation at the site or adjacent to the site of damaged kidney tissue. Injections can be administered as bolus injections or by continuous infusion. Depending on the route of administration, an agent can be coated with or disposed in a selected material to protect it from natural conditions which may detrimentally affect its ability to perform its intended function. An agent may be administered alone, or in conjunction with a pharmaceutically acceptable carrier. Further, an agent may be coadministered with a pharmaceutically acceptable carrier. An agent also may be administered as a prodrug, which is converted to its active form in vivo.

As used herein, the term “determining” refers to both quantitative and qualitative determinations and as such, the term “determining” is used interchangeably herein with “assaying,” “measuring,” and the like.

“Substantially purified” generally refers to isolation of a substance (e.g., compound, polynucleotide, protein, polypeptide, antibody, aptamer) such that the substance comprises the majority percent of the sample in which it resides. Typically in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

By “isolated” is meant, when referring to a polypeptide or peptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

By “therapeutically effective dose or amount” of pulsed focused ultrasound (pFUS) therapy, or mesenchymal stromal cells (MSCs) or MSC-derived extracellular vesicles is intended an amount that, when the pFUS and MSCs and/or MSC-derived extracellular vesicles are administered in combination, or when, in addition, a kidney therapeutic agent is administered, as described herein, brings about a positive therapeutic response, such as promoting regeneration or repair of damaged kidney tissue. Additionally, administering a treatment, as described herein, may reduce levels of inflammatory cytokines, decrease apoptosis, increase cell survival and proliferation in kidney tissue, upregulate expression of heat shock proteins such as HSP20 and HSP40, and/or decrease expression of HSP70 or HSP90, and/or promote activation of PI3K/Akt signaling and/or homing of MSCs to damaged kidney tissue, and/or suppress NLRP3 inflammasome activity and inflammasome-mediated inflammation. A therapeutically effective dose or amount can be administered in one or more administrations

An “effective amount” of an agent (e.g., small molecule, protein, polypeptide, peptide, fusion protein, hormone, transcription factor, nucleic acid, antibody, antibody mimetic, aptamer, or CRISPR system targeting, e.g., the HSP20 or HSP40 genes or genes involved in PI3K/Akt signaling (e.g., Cas9, Cas12a), RNA (Cas13), or the epigenome (dCas9 fusion protein) is an amount sufficient to increase expression of HSP20 or HSP40 and/or decrease expression of HSP70 or HSP90 and/or promote activation of PI3K/Akt signaling, and/or suppress NLRP3 inflammasome activity and inflammasome-mediated inflammation. An effective amount can be administered in one or more administrations, applications, or dosages.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

By “subject” is meant any member of the subphylum chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like.

The term “antibody” encompasses polyclonal antibodies, monoclonal antibodies as well as hybrid antibodies, altered antibodies, chimeric antibodies, and humanized antibodies. The term antibody includes: hybrid (chimeric) antibody molecules (see, for example, Winter et al. (1991) Nature 349:293-299; and U.S. Pat. No. 4,816,567); F(ab′)₂ and F(ab) fragments; Fᵥ molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (scFv) (see, e.g., Huston et al. (1988) Proc Natl Acad Sci USA 85:5879-5883); nanobodies or single-domain antibodies (sdAb) (see, e.g., Wang et al. (2016) Int J Nanomedicine 11 :3287-3303, Vincke et al. (2012) Methods Mol Biol 911 :15-26; dimeric and trimeric antibody fragment constructs; minibodies (see, e.g., Pack et al. (1992) Biochem 31 :1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

The phrase “specifically (or selectively) binds” with reference to binding of an antibody to an antigen refers to a binding reaction that is determinative of the presence of the antigen in a heterogeneous population of proteins and other biologics. Thus, under designated immunoassay conditions, the specified antibodies bind to a particular antigen at least two times the background and do not substantially bind in a significant amount to other antigens present in the sample. Specific binding to an antigen under such conditions may require an antibody that is selected for its specificity for a particular antigen. For example, antibodies raised to an antigen from specific species such as rat, mouse, or human can be selected to obtain only those antibodies that are specifically immunoreactive with the antigen and not with other proteins, except for polymorphic variants and alleles. This selection may be achieved by subtracting out antibodies that cross-react with molecules from other species. A variety of immunoassay formats may be used to select antibodies specifically immunoreactive with a particular antigen. For example, solid-phase ELISA immunoassays are routinely used to select antibodies specifically immunoreactive with a protein (see, e.g., Harlow & Lane. Antibodies, A Laboratory Manual (1988), for a description of immunoassay formats and conditions that can be used to determine specific immunoreactivity). Typically, a specific or selective reaction will be at least twice background signal or noise and more typically more than 10 to 100 times background.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms will be used interchangeably. Thus, these terms include, for example, 3'-deoxy-2',5'-DNA, oligodeoxyribonucleotide N3' P5' phosphoramidates, 2'-O-alkyl-substituted RNA, double-and single-stranded DNA, as well as double- and single-stranded RNA, microRNA, DNA:RNA hybrids, and hybrids between PNAs and DNA or RNA, and also include known types of modifications, for example, labels which are known in the art, methylation, “caps,” substitution of one or more of the naturally occurring nucleotides with an analog (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. The term also includes locked nucleic acids (e.g., comprising a ribonucleotide that has a methylene bridge between the 2'-oxygen atom and the 4'-carbon atom). See, for example, Kurreck et al. (2002) Nucleic Acids Res. 30: 1911-1918; Elayadi et al. (2001) Curr. Opinion Invest. Drugs 2: 558-561; Orum et al. (2001) Curr. Opinion Mol. Ther. 3: 239-243; Koshkin et al. (1998) Tetrahedron 54: 3607-3630; Obika et al. (1998) Tetrahedron Lett. 39: 5401-5404.

The term “transfection” is used to refer to the uptake of foreign DNA or RNA by a cell. A cell has been “transfected” when exogenous DNA or RNA has been introduced inside the cell membrane. A number of transfection techniques are generally known in the art. See, e.g., Graham et al. (1973) Virology, 52:456, Sambrook et al. (2001) Molecular Cloning, a laboratory manual, 3rd edition, Cold Spring Harbor Laboratories, New York, Davis et al. (1995) Basic Methods in Molecular Biology, 2nd edition, McGraw-Hill, and Chu et al. (1981) Gene 13:197. Such techniques can be used to introduce one or more exogenous DNA or RNA moieties into suitable host cells.

A “CRISPR system” refers collectively to transcripts and other elements involved in the expression of or directing the activity of CRISPR-associated (“Cas”) genes. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence.

The term “Cas9” as used herein encompasses type II clustered regularly interspaced short palindromic repeats (CRISPR) system Cas9 endonucleases from any species, and also includes biologically active fragments, variants, analogs, and derivatives thereof that retain Cas9 endonuclease activity (i.e., catalyze site-directed cleavage of DNA to generate double-strand breaks).

A Cas9 endonuclease binds to and cleaves DNA at a site comprising a sequence complementary to its bound guide RNA (gRNA). For purposes of Cas9 targeting, a gRNA may comprise a sequence “complementary” to a target sequence (e.g., major or minor allele), capable of sufficient base-pairing to form a duplex (i.e., the gRNA hybridizes with the target sequence). Additionally, the gRNA may comprise a sequence complementary to a PAM sequence, wherein the gRNA also hybridizes with the PAM sequence in a target DNA.

By “selectively binds” with reference to a guide RNA is meant that the guide RNA binds preferentially to a target sequence of interest or binds with greater affinity to the target sequence than to other genomic sequences. For example, a gRNA will bind to a substantially complementary sequence and not to unrelated sequences. A gRNA that “selectively binds” to a particular allele, such as a particular mutant allele (e.g., allele comprising a substitution, insertion, or deletion), denotes a gRNA that binds preferentially to the particular target allele, but to a lesser extent to a wild-type allele or other sequences. A gRNA that selectively binds to a particular target DNA sequence will selectively direct binding of Cas9 to a substantially complementary sequence at the target site and not to unrelated sequences.

The term “donor polynucleotide” refers to a polynucleotide that provides a sequence of an intended edit to be integrated into the genome at a target locus by homology directed repair (HDR).

A “target site” or “target sequence” is the nucleic acid sequence recognized (i.e., sufficiently complementary for hybridization) by a guide RNA (gRNA) or a homology arm of a donor polynucleotide. The target site may be allele-specific (e.g., a major or minor allele).

By “homology arm” is meant a portion of a donor polynucleotide that is responsible for targeting the donor polynucleotide to the genomic sequence to be edited in a cell. The donor polynucleotide typically comprises a 5' homology arm that hybridizes to a 5' genomic target sequence and a 3' homology arm that hybridizes to a 3' genomic target sequence flanking a nucleotide sequence comprising the intended edit to the genomic DNA. The homology arms are referred to herein as 5' and 3' (i.e., upstream and downstream) homology arms, which relates to the relative position of the homology arms to the nucleotide sequence comprising the intended edit within the donor polynucleotide. The 5' and 3' homology arms hybridize to regions within the target locus in the genomic DNA to be modified, which are referred to herein as the “5' target sequence” and “3' target sequence,” respectively. The nucleotide sequence comprising the intended edit is integrated into the genomic DNA by HDR or recombineering at the genomic target locus recognized (i.e., sufficiently complementary for hybridization) by the 5' and 3' homology arms.

“Administering” a nucleic acid to a cell comprises transducing, transfecting, electroporating, translocating, fusing, phagocytosing, shooting or ballistic methods, etc., i.e., any means by which a nucleic acid can be transported across a cell membrane.

### Administration of pFUS in Combination With MSCs or MSC-Derived Extracellular Vesicles

At least one therapeutically effective dose of pFUS therapy in combination with MSCs and/or MSC-derived extracellular vesicles will be administered to damaged kidney tissue. By “therapeutically effective dose or amount” of each of these agents is intended an amount that when administered in combination with the other agents, brings about a positive therapeutic response with respect to treatment of an individual for kidney damage, such as caused by an acute kidney injury or chronic kidney disease. For example, pFUS therapy in combination with MSCs and/or MSC-derived extracellular vesicles may be used to treat an acute kidney injury including, without limitation, kidney damage caused by chemotherapy, a chemical exposure to nephrotoxic agents, surgery, ischemia, a urinary tract obstruction, or a traumatic physical injury or chronic kidney disease including, without limitation, kidney damage such as caused by high blood pressure, diabetes, glomerulonephritis, or polycystic kidney disease. Of particular interest is an amount of pFUS therapy that improves kidney function, promotes regeneration of damaged kidney tissue, and/or suppresses NLRP3 inflammasome-mediated inflammation. Additionally, administering pFUS, as described herein, may be used to modulate gene expression and/or paracrine secretion (e.g., alter levels of pro-inflammatory cytokines, anti-inflammatory cytokines, growth factors, angiogenic factors, cell adhesion factors, and the like) or for homing of stem cells.

The MSCs may be derived from any source including, without limitation, bone marrow, adipose tissue, umbilical cord tissue, molar tooth bud tissue, and amniotic fluid. The MSCs may be obtained directly from the patient to be treated, a donor, a culture of cells from a donor, or from established cell culture lines.

The MSC-derived extracellular vesicles may include, without limitation, exosomes, ectosomes, microvesicles, or orther microparticles derived from the plasma membrane of MSCs.

The MSCs and/or MSC-derived extracellular vesicles may be administered in accordance with any medically acceptable method known in the art. For example, intra-arterial administration of MSC-derived extracellular vesicles via the renal artery can be performed as described in Example 2. Alternatively, MSCs and/or MSC-derived extracellular vesicles can be injected or surgically transplanted at a site of kidney damage or adjacent to a site of kidney damage.

The pFUS therapy can be administered prior to, concurrent with, or subsequent to the MSCs and/or MSC-derived extracellular vesicles. Thus, the three agents, or two of the three agents can be presented to the individual by way of concurrent therapy. By “concurrent therapy” is intended administration to a human subject such that the therapeutic effect of the combination is caused in the subject undergoing therapy. For example, concurrent therapy may be achieved by administering at least one therapeutically effective dose of pFUS and at least one therapeutically effective dose of a pharmaceutical composition comprising MSCs and/or MSC-derived extracellular vesicles. Similarly, the MSCs and/or MSC-derived extracellular vesicles, can be administered in at least one therapeutic dose. Administration of the pFUS and the MSCs and/or MSC-derived extracellular vesicles can be at the same time (i.e., simultaneously) or at different times (i.e., sequentially, in either order, on the same day, or on different days), as long as the therapeutic effect of the combination of these agents is caused in the subject undergoing therapy.

In certain embodiments, multiple therapeutically effective doses of pFUS therapy in combination with the MSCs and/or MSC-derived extracellular vesicles will be administered to the damaged kidney tissue. For example, a therapeutically effective dose can be administered, one day a week, two days a week, three days a week, four days a week, or five days a week, and so forth. By “intermittent” administration is intended the therapeutically effective dose can be administered, for example, every other day, every two days, every three days, and so forth. For example, in some embodiments, pFUS will be administered twice-weekly or thrice-weekly for an extended period of time, such as for 1, 2, 3, 4, 5, 6, 7, 8...10...15...24 weeks, and so forth. By “twice-weekly” or “two times per week” is intended that two therapeutically effective doses of pFUS is administered to the subject within a 7-day period, beginning on day 1 of the first week of administration, with a minimum of 72 hours, between doses and a maximum of 96 hours between doses. By “thrice weekly” or “three times per week” is intended that three therapeutically effective doses are administered to the subject within a 7-day period, allowing for a minimum of 48 hours between doses and a maximum of 72 hours between doses. For purposes of the present disclosure, this type of dosing is referred to as “intermittent” therapy. In accordance with the methods of the present invention, a subject can receive intermittent therapy (i.e., twice-weekly or thrice-weekly administration of a therapeutically effective dose) for one or more weekly cycles until the desired therapeutic response is achieved.

Multiple cycles of the combination therapy may be performed on a single region of the kidney or two or more different regions of the kidney. For example, multiple overlapping or non-overlapping regions in the kidney can be treated with the pFUS in combination with the MSCs and/or MSC-derived extracellular vesicles. In some embodiments, non-overlapping adjacent regions in the kidney are treated with the pFUS in combination with the MSCs and/or MSC-derived extracellular vesicles. In some embodiments, the combination therapy is administered for at least 1 week, at least 2 weeks, at least 3 weeks, or at least 4 weeks or longer until damaged kidney tissue is regenerated and/or kidney function is restored.

The combination therapy may be coupled with imaging guidance (e.g., ultrasound or magnetic resonance imaging) to correctly position the delivery of sound waves and the MSCs and/or MSC-derived extracellular vesicles and to avoid causing effects on intervening tissues. In some embodiments, imaging is used to focus sound waves within a relatively small focal zone (e.g., typically 1 mm × 1 mm × 10 mm) to treat particular cells (e.g., glomerulus parietal cells, glomerulus podocytes, proximal tubule brush border cells, loop of Henle thin segment cells, thick ascending limb cells, distal tubule cells, collecting duct principal cells, collecting duct intercalated cell, interstitial kidney cells) or structures within the kidney. For example, imaging can be used to focus sound waves at sites of kidney damage in need of treatment.

In certain embodiments, the pFUS is administered with an ultrasound frequency ranging from about 20 kHz to about 5.0 MHz, about 0.7 MHz to about 3.0 MHz, or about 1.0 MHz to about 1.1 MHz, including any ultrasound frequency within these ranges, such as 0.2, 0.4, 0.6, 0.8, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 3.0, 3.2, 3.4, 3.6, 3.8, 4.0, 4.2, 4.4, 4.6, 4.8, or 5.0 MHz.

In certain embodiments, the pFUS is administered with a pulse repetition frequency (PRF) ranging from 0.1 Hz to 1000 Hz, 1 Hz to 100 Hz, or about 5 Hz to 20 Hz, or any PRF with these ranges, such as 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, or 1000 Hz. In certain embodiments, the pFUS is administered with a PRF of about 5 Hz.

In certain embodiments, the pFUS is administered with an ultrasound duty cycle ranging from 0.01% to 100% or 1% to 20%, including any ultrasound duty cycle within these ranges such as 0.01%, 0.1 %, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 90%, 95%, or 100%. In some embodiments, the pFUS is administered with an ultrasound duty cycle of about 5%. In some embodiments, the pFUS therapy is administered with an ultrasound duty cycle of less than 1%.

In certain embodiments, the pFUS is administered with a negative peak pressure (NPP) ranging from 0.1 MPa to 10 MPa, including any NPP within this range such as 0.1, 0.2, 0.3, 0.4, 0.5, 0.6, 0.7, 0.8, 0.9, 1, 2, 3, 4, 5, 6, 7, 8, 9, or 10 MPa. In some embodiments, the pFUS is administered with a negative peak pressure (NPP) of up to 3 MPa. In some embodiments, the pFUS is administered with a negative peak pressure (NPP) of up to 3 MPa. In some embodiments, the NPP is about 2.9 MPa.

In certain embodiments, the pFUS is administered to the subject for a time ranging from about 20 seconds to about 7 minutes, including any amount of time within this range, such as 20 seconds, 25 seconds, 30 seconds, 35 seconds, 40 seconds, 45 seconds, 50 seconds, 55 seconds, 1 minute, 1.25 minutes, 1.5 minutes, 1.75 minutes, 2 minutes, 2.25 minutes, 2.5 minutes, 2.75 minutes, 3 minutes, 3.25 minutes, 3.5 minutes, 3.75 minutes, 4 minutes, 4.25 minutes, 4.5 minutes, 4.75 minutes, 5 minutes, 5.25 minutes,, 5.5 minutes, 5.75 minutes, 6 minutes, 6.25 minutes, 6.5 minutes, 6.75 minutes, or 7 minutes. In some embodiments, the pFUS therapy is administered to the subject for at least 20 seconds. In some embodiments, the pFUS therapy is administered to the subject for a period ranging from about 1 minute to about 5 minutes. In one embodiment, the pFUS therapy is administered to the subject for about 160 seconds.

In certain embodiments, the pFUS is administered with a spatial average pulse average intensity (Iₛₐₚₐ) of about 272 W/cm².

In certain embodiments, pFUS is administered with a pulse length of about 10 milliseconds.

### Screening Agents for Treating Kidney Damage

Screening methods are provided for identifying candidate agents that increase expression of HSP20 or HSP40, decrease expression of HSP70 or HSP90, increase activation of PI3K/Akt signaling, or decrease NLRP3 inflammasome activity, which may be useful in treating kidney damage. Candidate agents include, without limitation, small molecules, i.e., drugs, genetic constructs that increase or decrease expression of an RNA of interest, anti-sense nucleic acids, siRNAs, miRNAs, CRISPR systems, anti-inflammatory agents, transcription factors, hormones, hormone antagonists, and the like. A variety of assays may be used for this purpose, and in many embodiments, a candidate agent will be tested in different assays to confirm efficacy in treating kidney damage. For example, biochemical assays may determine the ability of an agent to inhibit or activate biological activity or decrease or increase gene expression. In addition, cell-based assays may be used, for example, for testing for gene expression or growth or proliferation of kidney cells in the absence or presence of a candidate agent.

Candidate agents of interest are biologically active agents that encompass numerous chemical classes, primarily organic molecules, which may include organometallic molecules, inorganic molecules, genetic sequences, etc. In particular, candidate drugs, select therapeutic antibodies and protein-based therapeutics with preferred biological response functions can be evaluated. Candidate agents comprise functional groups necessary for structural interaction with proteins, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups.

Candidate agents are also found among biomolecules, including peptides, polynucleotides, saccharides, fatty acids, steroids, purines, pyrimidines, derivatives, structural analogs or combinations thereof. Assays may further include suitable controls (e.g., a sample in the absence of the test agent). Generally, a plurality of assay mixtures is run in parallel with different agent concentrations to obtain a differential response to the various concentrations. Typically, one of these concentrations serves as a negative control, i.e., at zero concentration or below the level of detection.

A variety of other reagents may be included in the screening assay. These include reagents like salts, neutral proteins, e.g., albumin, detergents, etc., including agents that are used to facilitate optimal binding activity and/or reduce non-specific or background activity. Reagents that improve the efficiency of the assay, such as protease inhibitors, nuclease inhibitors, antimicrobial agents, etc. may be used. The components of the assay mixture are added in any order that provides for the requisite activity. Incubations are performed at any suitable temperature, typically between 4° C. and 40° C. Incubation periods are selected for optimum activity but may also be optimized to facilitate rapid high-throughput screening. In some embodiments, between 0.1 hour and 1 hour, between 1 hour and 2 hours, or between 2 hours and 4 hours, will be sufficient.

Test agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Moreover, screening may be directed to known pharmacologically active compounds and chemical analogs thereof, or to new agents with unknown properties such as those created through rational drug design.

In some embodiments, test agents are synthetic compounds. A number of techniques are available for the random and directed synthesis of a wide variety of organic compounds and biomolecules, including expression of randomized oligonucleotides. See for example WO 94/24314, hereby expressly incorporated by reference, which discusses methods for generating new compounds, including random chemistry methods as well as enzymatic methods.

In another embodiment, the test agents are provided as libraries of natural compounds in the form of bacterial, fungal, plant and animal extracts that are available or readily produced. Additionally, natural or synthetically produced libraries and compounds are readily modified through conventional chemical, physical and biochemical means. Known pharmacological agents may be subjected to directed or random chemical modifications, including enzymatic modifications, to produce structural analogs.

In some embodiments, the test agents are organic moieties. In this embodiment, test agents are synthesized from a series of substrates that can be chemically modified. “Chemically modified” herein includes traditional chemical reactions as well as enzymatic reactions. These substrates generally include, but are not limited to, alkyl groups (including alkanes, alkenes, alkynes and heteroalkyl), aryl groups (including arenes and heteroaryl), alcohols, ethers, amines, aldehydes, ketones, acids, esters, amides, cyclic compounds, heterocyclic compounds (including purines, pyrimidines, benzodiazepins, beta-lactams, tetracylines, cephalosporins, and carbohydrates), steroids (including estrogens, androgens, cortisone, ecodysone, etc.), alkaloids (including ergots, vinca, curare, pyrollizdine, and mitomycines), organometallic compounds, hetero-atom bearing compounds, amino acids, and nucleosides. Chemical (including enzymatic) reactions may be done on the moieties to form new substrates or candidate agents which can then be tested using the present invention.

In some embodiments test agents are assessed for any cytotoxic activity it may exhibit toward a living eukaryotic cell, using well-known assays, such as trypan blue dye exclusion, an MTT (3-(4,5-dimethylthiazol-2-yl)-2,5-diphenyl-2 H-tetrazolium bromide) assay, and the like. Agents that do not exhibit significant cytotoxic activity are considered candidate agents.

In some embodiments, a test agent that that increases expression of HSP20 or HSP40, decreases expression of HSP70 or HSP90, increases activation of PI3K/Akt signaling, or decreases NLRP3 inflammasome activity is further tested for its ability to promote kidney regeneration in a cell-based or tissue assay. In these embodiments, a test agent of interest is contacted with the kidney cells or tissue; and the effect, if any, of the test agent on the kidney cells or tissue is determined.

For example, a population of kidney cells can be cultured in vitro in the presence of an effective dose of an agent. The effect on growth, proliferation, and/or tissue regeneration may be assayed. The agent is added to the culture medium, and the culture medium is maintained under conventional conditions suitable for growth of the prostate cancer cells. Various commercially available systems have been developed for the growth of mammalian cells to provide for removal of adverse metabolic products, replenishment of nutrients, and maintenance of oxygen. By employing these systems, the medium may be maintained as a continuous medium, so that the concentrations of the various ingredients are maintained relatively constant or within a prescribed range.

In addition, an agent that increases expression of HSP20 or HSP40, decreases expression of HSP70 or HSP90, increases activation of PI3K/Akt signaling, or decreases NLRP3 inflammasome activity, identified as described herein, can be tested in an animal model to determine its efficacy in promoting kidney regeneration and/or restoring kidney function as well as the toxicity or side effects of treatment with such an agent. Alternatively, an agent identified, as described herein, can be used in an animal model to determine the mechanism of action of such an agent. Monitoring the efficacy of agents (e.g., drugs) in treating kidney damage can be applied not only in basic drug screening, but also in clinical trials. Furthermore, this disclosure pertains to uses of novel agents identified by the above-described screening assays for treatment of kidney damage.

Candidate agents that increase expression of HSP20 or HSP40 or decrease expression of HSP70 or HSP90 can be identified by contacting a cell with a candidate compound and measuring the expression of HSP20, HSP40, HSP70 or HSP90 as determined by e.g., mRNA or polypeptide levels. The level of expression of mRNA or protein in the presence of the candidate compound is compared to the level of expression of mRNA or protein in the absence of the candidate compound. The candidate compound can then be identified based on this comparison. For example, when expression of mRNA or protein in cells is decreased (statistically significantly less) in the presence of the candidate compound than in its absence, the candidate compound is identified as an agent that inhibits gene expression. Alternatively, when expression of mRNA or protein is increased (statistically significantly more) in the presence of the candidate compound than in its absence, the candidate compound is identified as an agent that activates gene expression.

Any convenient protocol may be used for evaluating gene expression by detecting protein or mRNA levels in the presence or absence of a candidate agent. For measuring protein levels in a sample, various antibody-based methods, including without limitation immunoassays, e.g., enzyme-linked immunosorbent assays (ELISAs), immunohistochemistry, and flow cytometry (FACS) may be used. Any convenient antibody can be used that specifically binds to the protein. The terms “specifically binds” or “specific binding” as used herein refer to preferential binding to a molecule relative to other molecules or moieties in a solution or reaction mixture (e.g., an antibody specifically binds to a particular polypeptide or epitope relative to other available polypeptides or epitopes). In some embodiments, the affinity of one molecule for another molecule to which it specifically binds is characterized by a K_{d} (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less). By “Affinity” it is meant the strength of binding, increased binding affinity being correlated with a lower K_{d}.

While a variety of different manners of assaying for protein levels are known in the art, one representative and convenient type of protocol for assaying protein levels is the enzyme-linked immunosorbent assay (ELISA). In ELISA and ELISA-based assays, one or more antibodies specific for the proteins of interest may be immobilized onto a selected solid surface, preferably a surface exhibiting a protein affinity such as the wells of a polystyrene microtiter plate. After washing to remove incompletely adsorbed material, the assay plate wells are coated with a non-specific “blocking” protein that is known to be antigenically neutral with regard to the test sample such as bovine serum albumin (BSA), casein or solutions of powdered milk. This allows for blocking of non-specific adsorption sites on the immobilizing surface, thereby reducing the background caused by non-specific binding of antigen onto the surface. After washing to remove unbound blocking protein, the immobilizing surface is contacted with the sample to be tested under conditions that are conducive to immune complex (antigen/antibody) formation. Such conditions include diluting the sample with diluents such as BSA or bovine gamma globulin (BGG) in phosphate buffered saline (PBS)/Tween or PBS/Triton-X 100, which also tend to assist in the reduction of nonspecific background, and allowing the sample to incubate for about 2-4 hours at temperatures on the order of about 25°-27° C. (although other temperatures may be used). Following incubation, the antisera-contacted surface is washed so as to remove non-immunocomplexed material. An exemplary washing procedure includes washing with a solution such as PBS/Tween, PBS/Triton-X 100, or borate buffer. The occurrence and amount of immunocomplex formation may then be determined by subjecting the bound immunocomplexes to a second antibody having specificity for the target that differs from the first antibody and detecting binding of the second antibody. In certain embodiments, the second antibody will have an associated enzyme, e.g., urease, peroxidase, or alkaline phosphatase, which will generate a color precipitate upon incubating with an appropriate chromogenic substrate. For example, a urease or peroxidase-conjugated anti-human IgG may be employed, for a period of time and under conditions which favor the development of immunocomplex formation (e.g., incubation for 2 hours at room temperature in a PBS-containing solution such as PBS/Tween). After such incubation with the second antibody and washing to remove unbound material, the amount of label is quantified, for example by incubation with a chromogenic substrate such as urea and bromocresol purple in the case of a urease label or 2,2'-azino-di-(3-ethyl-benzthiazoline)-6-sulfonic acid (ABTS) and H₂O₂, in the case of a peroxidase label. Quantitation is then achieved by measuring the degree of color generation, e.g., using a visible spectrum spectrophotometer. The preceding format may be altered by first binding the sample to the assay plate. Then, primary antibody is incubated with the assay plate, followed by detecting of bound primary antibody using a labeled second antibody with specificity for the primary antibody.

The solid substrate upon which the antibody or antibodies are immobilized can be made of a wide variety of materials and in a wide variety of shapes, e.g., microtiter plate, microbead, dipstick, resin particle, etc. The substrate may be chosen to maximize signal to noise ratios, to minimize background binding, as well as for ease of separation and cost. Washes may be effected in a manner most appropriate for the substrate being used, for example, by removing a bead or dipstick from a reservoir, emptying or diluting a reservoir such as a microtiter plate well, or rinsing a bead, particle, chromatographic column or filter with a wash solution or solvent.

Alternatively, non-ELISA based-methods for measuring the levels of a protein in a sample may be employed, and any convenient method may be used. Representative examples known to one of ordinary skill in the art include but are not limited to other immunoassay techniques such as radioimmunoassays (RIA), sandwich immunoassays, fluorescent immunoassays, enzyme multiplied immunoassay technique (EMIT), capillary electrophoresis immunoassays (CEIA), and immunoprecipitation assays; mass spectrometry, or tandem mass spectrometry, proteomic arrays, xMAP microsphere technology, western blotting, immunohistochemistry, flow cytometry, cytometry by time-of-flight (CyTOF), multiplexed ion beam imaging (MIBI), and detection in body fluid by electrochemical sensor. In, for example, flow cytometry methods, the quantitative level of gene products of the one or more genes of interest are detected on cells in a cell suspension by lasers. As with ELISAs and immunohistochemistry, antibodies (e.g., monoclonal antibodies) that specifically bind the polypeptides encoded by the genes of interest are used in such methods.

As another example, electrochemical sensors may be employed. In such methods, a capture aptamer or an antibody that is specific for a target protein (the “analyte”) is immobilized on an electrode. A second aptamer or antibody, also specific for the target protein, is labeled with, for example, pyrroquinoline quinone glucose dehydrogenase ((PQQ)GDH). The sample of body fluid is introduced to the sensor either by submerging the electrodes in body fluid or by adding the sample fluid to a sample chamber, and the analyte allowed to interact with the labeled aptamer/antibody and the immobilized capture aptamer/antibody. Glucose is then provided to the sample, and the electric current generated by (PQQ)GDH is observed, where the amount of electric current passing through the electrochemical cell is directly related to the amount of analyte captured at the electrode.

Flow cytometry can be used to distinguish subpopulations of cells expressing different cellular markers and to determine their frequency in a population of cells. Typically, whole cells are incubated with antibodies that specifically bind to the cellular markers. The antibodies can be labeled, for example, with a fluorophore, isotope, or quantum dot to facilitate detection of the cellular markers. The cells are then suspended in a stream of fluid and passed through an electronic detection apparatus. In addition, fluorescence-activated cell sorting (FACS) can be used to sort a heterogeneous mixture of cells into separate containers. (See, e.g., Shapiro Practical Flow Cytometry, Wiley-Liss, 4^{th} edition, 2003; Loken Immunofluorescence Techniques in Flow Cytometry and Sorting, Wiley, 2^{nd} edition, 1990; Flow Cytometry: Principles and Applications, (ed. Macey), Humana Press 1^{st} edition, 2007; herein incorporated by reference in their entireties.)

In other embodiments, the amount or level in the sample of mRNA encoded by a gene is determined. Any convenient method for measuring mRNA levels in a sample may be used, e.g., hybridization-based methods, e.g., northern blotting and in situ hybridization (Parker & Barnes, Methods in Molecular Biology 106:247-283 (1999)), RNase protection assays (Hod, Biotechniques 13:852-854 (1992)), and PCR-based methods (e.g., reverse transcription PCR (RT-PCR) (Weis et al., Trends in Genetics 8:263-264 (1992)).

For measuring mRNA levels, the starting material may be total RNA, i.e., unfractionated RNA, or poly A+ RNA isolated from a suspension of cells (e.g., prostate cancer cells). General methods for mRNA extraction are well known in the art and are disclosed in standard textbooks of molecular biology, including Ausubel et al., Current Protocols of Molecular Biology, John Wiley and Sons (1997). RNA isolation can also be performed using a purification kit, buffer set and protease from commercial manufacturers, according to the manufacturer’s instructions. For example, RNA from cell suspensions can be isolated using Qiagen RNeasy mini-columns, and RNA from cell suspensions or homogenized tissue samples can be isolated using the TRIzol reagent-based kits (Invitrogen), MasterPure Complete DNA and RNA Purification Kit (EPICENTRE, Madison, Wis.), Paraffin Block RNA Isolation Kit (Ambion, Inc.) or RNA Stat-60 kit (Tel-Test).

The mRNA levels may be measured by any convenient method. Examples of methods for measuring mRNA levels may be found in, e.g., the field of differential gene expression analysis. One representative and convenient type of protocol for measuring mRNA levels is array-based gene expression profiling. Such protocols are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively.

Specific hybridization technology which may be employed in the subject methods includes that described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the phenotype determinative genes whose expression is being assayed is contacted with target nucleic acids as described above. Contact is carried out under hybridization conditions, e.g., stringent hybridization conditions, and unbound nucleic acid is then removed. The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

The resultant pattern of hybridized nucleic acid provides information regarding expression for each of the genes that have been probed, where the expression information is in terms of whether or not the gene is expressed and, typically, at what level, where the expression data, i.e., expression profile (e.g., in the form of a transcriptosome), may be both qualitative and quantitative.

Additionally or alternatively, non-array-based methods for quantitating the level of one or more nucleic acids in a sample may be employed. These include those based on amplification protocols, e.g., Polymerase Chain Reaction (PCR)-based assays, including quantitative PCR, reverse-transcription PCR (RT-PCR), real-time PCR, and the like, e.g., TaqMan, RT-PCR, MassARRAY System, BeadArray technology, and Luminex technology; and those that rely upon hybridization of probes to filters, e.g., Northern blotting and in situ hybridization. Serial Analysis Gene Expression (SAGE) can also be used to determine RNA abundances in a cell sample. See, e.g., Velculescu et al., 1995, Science 270:484-7; Carulli, et al., 1998, Journal of Cellular Biochemistry Supplements 30/31 :286-96; herein incorporated by reference in their entireties. SAGE analysis does not require a special device for detection, can be used for simultaneously detecting the expression of large numbers of transcription products.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-52 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. A method of treating damaged kidney tissue in a subject, the method comprising locally administering to the damaged kidney tissue a therapeutically effective amount of pulsed focused ultrasound (pFUS) therapy in combination with a therapeutically effective amount of mesenchymal stromal cells (MSCs) or MSC-derived extracellular vesicles.
2. The method of aspect 1, wherein the MSCs or the MSC-derived extracellular vesicles are administered locally to the damaged kidney tissue intra-arterially via a renal artery.
3. The method of aspect 1 or 2, wherein the pFUS therapy is administered with an ultrasound frequency ranging from 20 kHz to 5.0 MHz.
4. The method of aspect 3, wherein the pFUS therapy is administered with an ultrasound frequency ranging from 0.7 MHz to 3.0 MHz.
5. The method of aspect 4, wherein the ultrasound frequency ranges from about 1.0 MHz to about 1.1 MHz.
6. The method of any one of aspects 1 to 5, wherein the pFUS therapy is administered with a pulse repetition frequency (PRF) of about 5 Hz.
7. The method of any one of aspects 1 to 6, wherein the pFUS therapy is administered with an ultrasound duty cycle ranging from 0.01% to 100%.
8. The method of aspect 7, wherein the pFUS therapy is administered with an ultrasound duty cycle ranging from 1% to 20%.
9. The method of aspect 8, wherein the pFUS therapy is administered with an ultrasound duty cycle of about 5%.
10. The method of any one of aspects 1 to 9, wherein the pFUS therapy is administered with a negative peak pressure (NPP) ranging from 0.1 MPa to 10 MPa.
11. The method of aspect 10, wherein the NPP is about 2.9 MPa.
12. The method of any one of aspects 1 to 11, wherein the pFUS therapy is administered with a spatial average pulse average intensity (Iₛₐₚₐ) of about 272 W/cm².
13. The method of any one of aspects 1 to 12, wherein the pFUS therapy is administered to the subject for at least 30 seconds.
14. The method of aspect 13, wherein the pFUS therapy is administered to the subject for a period ranging from about 1 minute to about 5 minutes.
15. The method of any one of aspects 1 to 14, wherein the pFUS therapy is administered at a single location or at multiple locations in the kidney.
16. The method of any one of aspects 1 to 15, further comprising imaging the damaged kidney tissue.
17. The method of aspect 16, wherein said imaging is performed by ultrasound, magnetic resonance imaging (MRI), computed tomography (CT), or scintigraphy.
18. The method of any one of aspects 1 to 17, wherein the MSCs are from bone marrow or adipose tissue.
19. The method of any one of aspects 1 to 18, wherein the kidney tissue is damaged from an acute kidney injury or chronic kidney disease.
20. The method of aspect 19, wherein the acute kidney injury is caused by chemotherapy, a chemical exposure, surgery, or a traumatic physical injury.
21. The method of any one of aspects 1 to 20, wherein the MSC-derived extracellular vesicles are exosomes or microvesicles.
22. The method of aspect 21, wherein the MSC-derived exosomes comprise one or more surface markers selected from the group consisting of CD9, CD63, and TSG101.
23. The method of aspect 22, wherein the MSC-derived exosomes comprise the surface markers: CD9, CD63, and TSG101.
24. The method of any one of aspects 1 to 23, wherein the MSC-derived extracellular vesicles have diameters ranging from about 20 nm to 180 nm.
25. The method of aspect 24, wherein the MSC-derived extracellular vesicles have a mean diameter of 118 nm.
26. The method of any one of aspects 1 to 25, wherein multiple cycles of treatment are administered to the subject.
27. A method of suppressing NLRP3 inflammasome-mediated inflammation in a subject, the method comprising locally administering to damaged kidney tissue an effective amount of pulsed focused ultrasound (pFUS) in combination with an effective amount of mesenchymal stromal cells (MSCs) or MSC-derived extracellular vesicles.
28. The method of aspect 27, wherein the MSCs or the MSC-derived extracellular vesicles are administered locally to the damaged kidney tissue intra-arterially via a renal artery.
29. The method of aspect 27 or 28, wherein the pFUS is administered with an ultrasound frequency ranging from 20 kHz to 5.0 MHz.
30. The method of aspect 29, wherein the pFUS is administered with an ultrasound frequency ranging from 0.7 MHz to 3.0 MHz.
31. The method of aspect 30, wherein the ultrasound frequency ranges from about 1.0 MHz to about 1.1 MHz.
32. The method of any one of aspects 27 to 31, wherein the pFUS is administered with a pulse repetition frequency (PRF) of about 5 Hz.
33. The method of any one of aspects 27 to 32, wherein the pFUS is administered with an ultrasound duty cycle ranging from 0.01% to 100%.
34. The method of aspect 33, wherein the pFUS is administered with an ultrasound duty cycle ranging from 1% to 20%.
35. The method of aspect 34, wherein the pFUS is administered with an ultrasound duty cycle of about 5%.
36. The method of any one of aspects 27 to 35, wherein the pFUS is administered with a negative peak pressure (NPP) ranging from 0.1 MPa to 10 MPa.
37. The method of aspect 36, wherein the NPP is about 2.9 MPa.
38. The method of any one of aspects 27 to 37, wherein the pFUS is administered with a spatial average pulse average intensity (Iₛₐₚₐ) of about 272 W/cm².
39. The method of any one of aspects 27 to 38, wherein the pFUS is administered to the subject for at least 30 seconds.
40. The method of aspect 39, wherein the pFUS is administered to the subject for a period ranging from about 1 minute to about 5 minutes.
41. The method of any one of aspects 27 to 40, wherein the pFUS therapy is administered at a single location or at multiple locations in the kidney.
42. A method of screening a candidate agent for treating kidney damage, the method comprising:
   a) contacting a test population of kidney cells or kidney tissue from a damaged kidney with the candidate agent; and
   b) measuring expression of HSP20, HSP40, HSP70, or HSP90, PI3K/Akt signaling, or NLRP3 inflammasome activity in the test population of kidney cells or kidney tissue, wherein increased expression of HSP20 or HSP40, decreased expression of HSP70 or HSP90, increased activation of PI3K/Akt signaling, or decreased NLRP3 inflammasome activity in the test population of kidney cells or kidney tissue compared to that in a negative control population of kidney cells or kidney tissue that are not contacted with the candidate agent indicates that the candidate agent may be useful for treating the kidney damage.
43. The method of aspect 42, wherein the test population of kidney cells or kidney tissue is obtained from a kidney damaged by an acute kidney injury or chronic kidney disease.
44. The method of aspect 42 or 43, wherein the candidate agent is a small molecule, a peptide, a protein, an aptamer, an antibody, an antibody mimetic, a transcription factor, a hormone, a nucleic acid, or a clustered regularly interspaced short palindromic repeats (CRISPR) system.
45. The method of aspect 44, wherein the CRISPR system targets a HSP20 gene, a HSP40 gene, a HSP70 gene, or a HSP90 gene; or a HSP20 RNA transcript, a HSP40 RNA transcript, a HSP70 RNA transcript, or a HSP90 RNA transcript; or makes epigenetic changes that increase expression of HSP20 or HSP40 or decrease expression of HSP70 or HSP90.
46. The method of aspect 45, wherein the CRISPR system comprises Cas9, Cas12a, Cas12d, Cas13a, Cas13b, Cas13d, or a dead Cas9 (dCas9).
47. A kidney therapeutic agent identified by the method of any one of aspects 42 to 46.
48. A composition comprising the kidney therapeutic agent of aspect 47 and a pharmaceutically acceptable excipient.
49. The composition of aspect 48, further comprising a pharmaceutically acceptable carrier selected from the group consisting of a cream, emulsion, gel, liposome, nanoparticle, or ointment.

### Experimental

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1 Reversing Acute Kidney Injury Following Pulsed Focused Ultrasound and Mesenchymal Stromal Cell Therapy: A Role for Heat Shock Protein-Mediated Pi3k/Akt Signaling

### Introduction

One promising method for improving the homing of MSCs, and potentiating their effect, is the use of pulsed focused ultrasound (pFUS). pFUS non-destructively sonicates target tissues with short-duration but high-intensity bursts of sound waves, eliciting a range of biological effects. Interestingly, pFUS can promote a transient local increase in cytokines, chemokines and trophic factors in the sonicated tissue,²³ some of which act as a beacon for circulating MSCs. In keeping with this mechanism, studies have shown that pFUS can enhance MSC homing to sonicated tissues, in both healthy murine skeletal muscle ²⁴ and the kidney.²⁵ Indeed, in a mouse model of cisplatin-induced AKI, pFUS enhanced the therapeutic effect of BM-MSCs (i.e. by improving renal function and mouse survival), by enhancing their homing, permeability, and retention in the damaged kidney.²⁶ Further studies then suggested that pFUS upregulated IFN-y in the injured kidney, which assists in BM-MSC migration and stimulates BM-MSCs to produce the anti-inflammatory cytokine IL-10.²⁷

However, other work has suggested that homing of BM-MSCs to the kidneys may not be necessary for improving kidney repair, as MSCs may act from distant sites via an endocrine mechanism of action, wherein they secrete factors that limit apoptosis and enhance the proliferation of the endogenous tubular cells.²⁸ Hence, the exact mechanism(s) by which BM-MSCs or pFUS can modulate kidney repair in the setting of AKI are not definitively known. In this study, we therefore induced AKI in mice by administering cisplatin and then examined the effect of BM-MSCs alone, pFUS alone, or combined pFUS + BM-MSCs treatment in order to compare the effectiveness of these three approaches in reversing renal damage and restoring renal function.

### Results

### Characterization of BM-MSCs

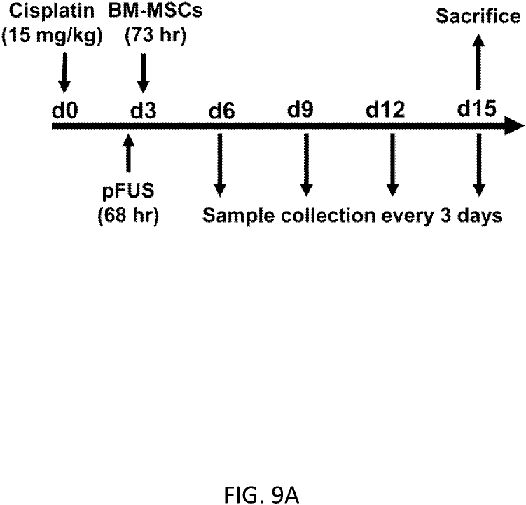

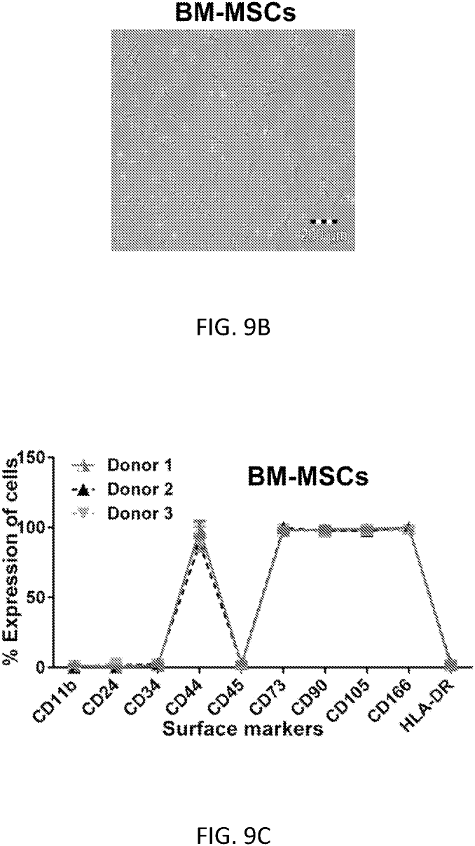

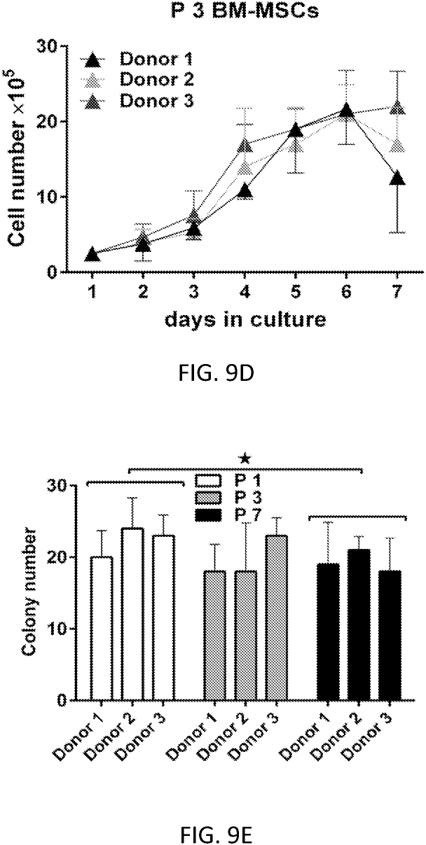

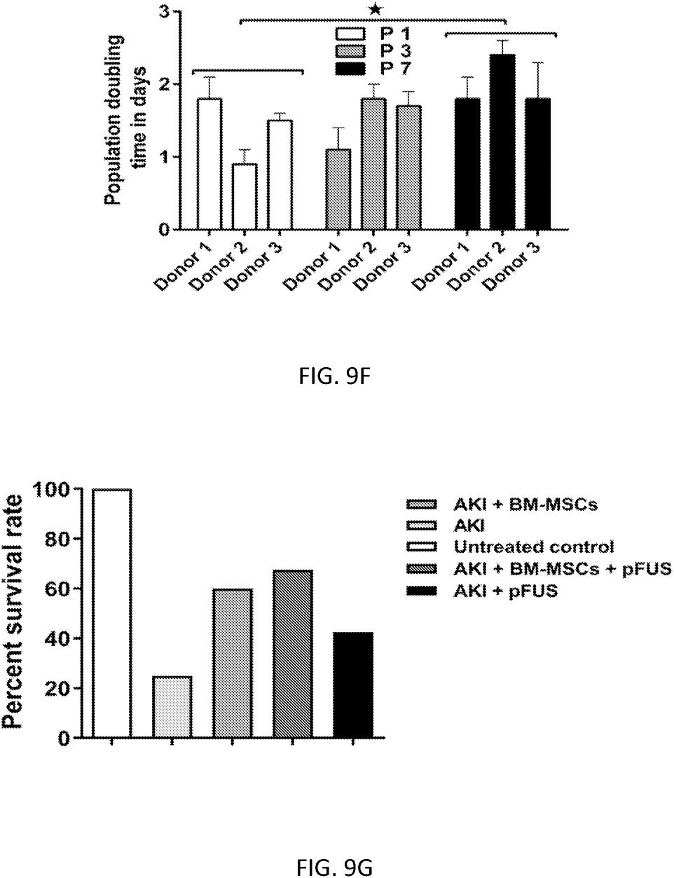

In the present study, we used a pooled sample of BM-MSCs collected from 3 different human donors. These human BM-MSCs exhibited mesenchymal stromal cell morphology with positive expression of the following surface markers: CD44, CD73, CD90, CD105 and CD166 and negative expression of CD11 b, CD24 and CD45 (FIGS. 9B, 9C). BM-MSCs from passage number 3 (P3) were used for all studies, based on their doubling time and colony formation rate (FIGS. 9D-9F).

### The Biological Effects of BM-MSCs and pFUS in the Setting of AKI

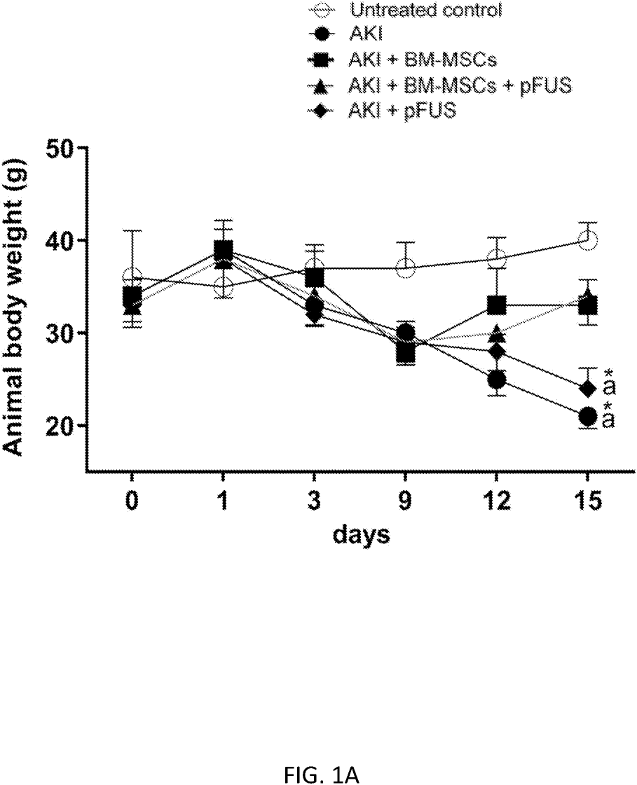

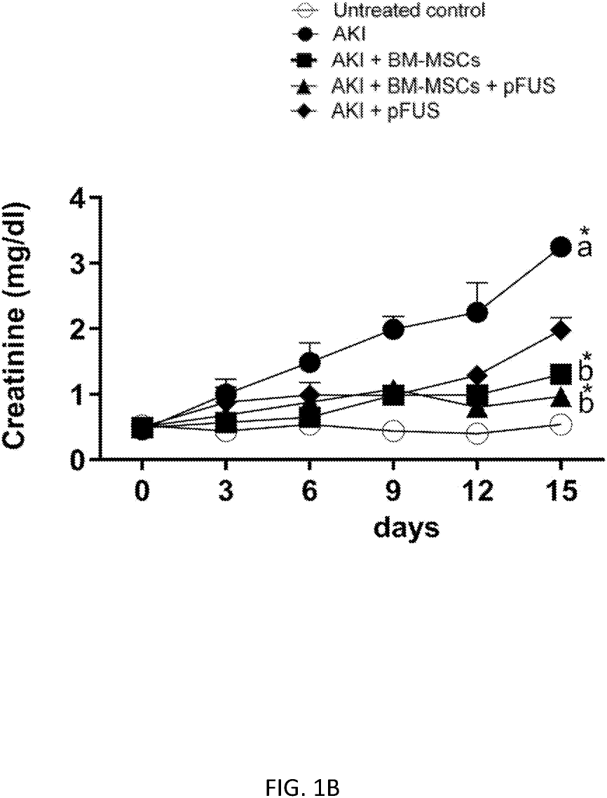

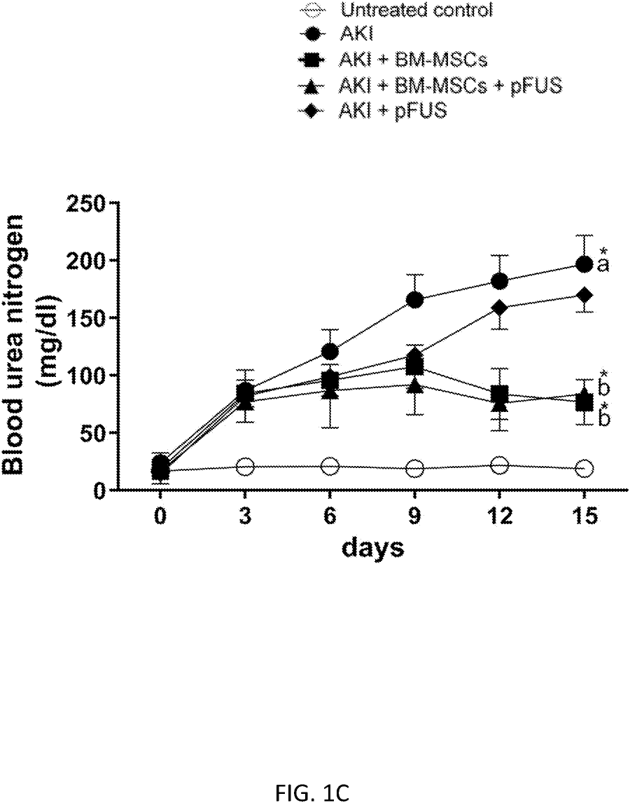

Following administration of cisplatin, there was a significant reduction in body weight in animals in the AKI group, compared to untreated controls; however, this effect was ameliorated in those animals which received treatment with either BM-MSCs alone or pFUS + BM-MSCs. pFUS alone did not improve body weight reduction (FIG. 1A). By day 3, there was a rise in blood creatinine levels in all animals following cisplatin administration. By day 15, animals in the AKI group showed significantly increased levels of blood creatinine compared to untreated controls (AKI: 3.25±0.08 vs. 0.53±0.11 mg/dl, p<0.05). Treatment with pFUS alone demonstrated decreased blood creatinine levels when compared to AKI group animals, though it did not reach statistical significance (pFUS: 1.98±0.19 vs. 3.25±0.08 mg/dl, p>0.05). In contrast, animals with AKI that were treated with either BM-MSCs alone or combined pFUS + BM-MSCs demonstrated a significant decrease in blood creatinine levels compared to the AKI group (BM-MSCs: 1.30±0.13 vs. 3.25±0.08 mg/dl, p<0.05; pFUS + BM-MSCs: 0.96±0.12 vs. 3.25±0.08, p>0.05) (FIG. 1B). Furthermore, at day 3, there was a significant rise in blood urea nitrogen (BUN) levels in all animals treated with cisplatin compared to the untreated control group (AKI: 196.77±35.47 vs. 18.79±1.67 mg/dL, p<0.05). While pFUS alone was able to somewhat decrease BUN compared to the AKI group, it did not reach statistical significance (169.88±7.99 vs. 196.77±35.47 mg/dL, p>0.05). Treatment with BM-MSCs in animals with AKI demonstrated a significant decrease in BUN levels compared to the AKI group (76.77±9.53 vs. 196.77±35.47, p<0.05) and a similar trend was also seen in those animals with AKI treated with pFUS + BM-MSCs (83.77±12.82 vs. 196.77±35.47 mg/dl, p<0.05). However, these values were still significantly higher when compared to untreated control animals (18.79±1.67 mg/dl) (FIG. 1C).

### Homing of BM-MSCs to the Kidney in the Setting of AKI

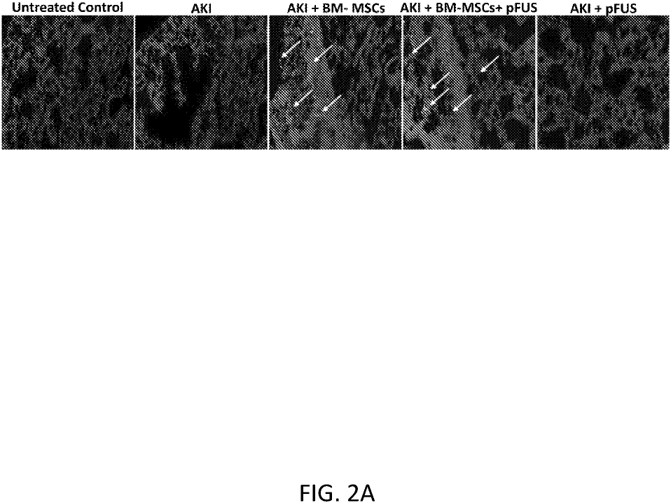

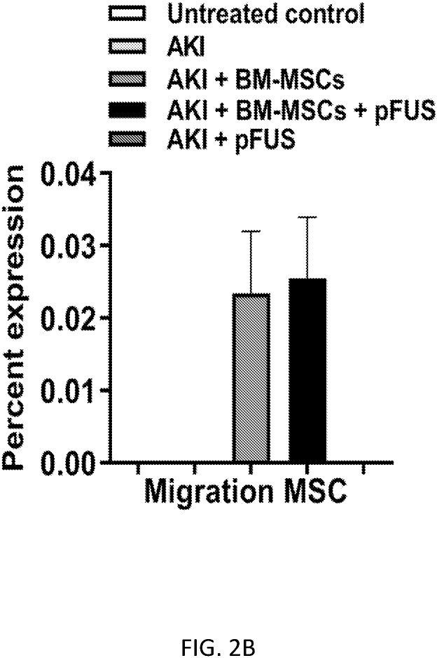

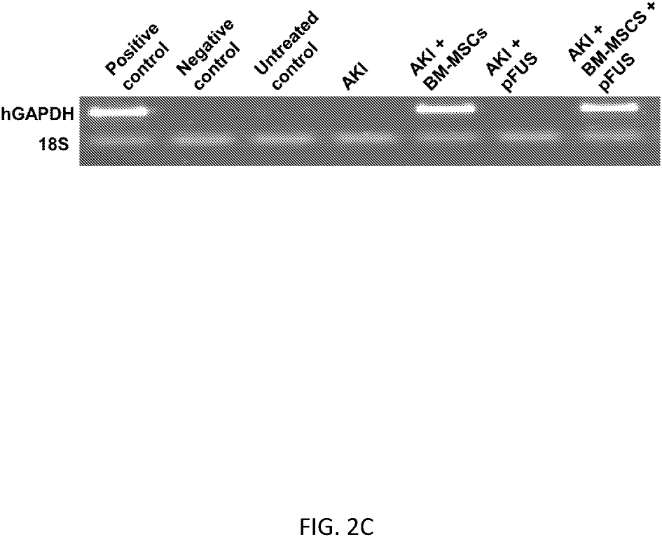

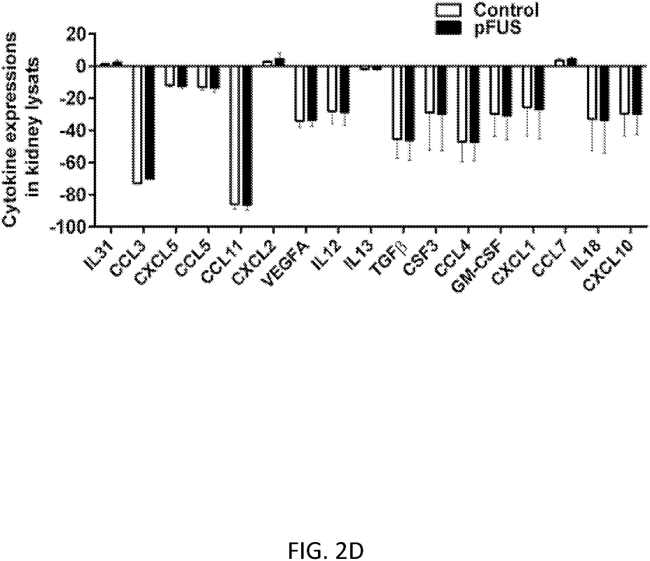

Following the development of AKI in mice, IV-administered GFP-labelled BM-MSCs homed to the injured kidney, as detected by immunofluorescence (IF) (FIG. 2A), flow cytometry (FIG. 2B), and qPCR for human GAPDH (FIG. 2C). However, there was no significant increase in the amount of BM-MSCs in the kidney in those animals treated with BM-MSCs + pFUS as demonstrated by only a minimal increase in the number of GFP-labelled BM-MSCs visible on IF, (688±50 vs. 710±80 per field, p>0.05) (FIG. 2A), percentage of GFP-positive cells detected on flow cytometry (0.023±0.009 vs. 0.025±0.009, p>0.05) (FIG. 2B) and human GAPDH expression by qPCR (FIG. 2C). Furthermore, there was no difference in the cytokine/chemoattractant expression between control kidneys and kidneys treated with pFUS, measured 48 hours after sonication (FIG. 2D).

### Histological and Structural Remodeling of the Acutely Injured Kidney Following BM-MSC and pFUS Treatment

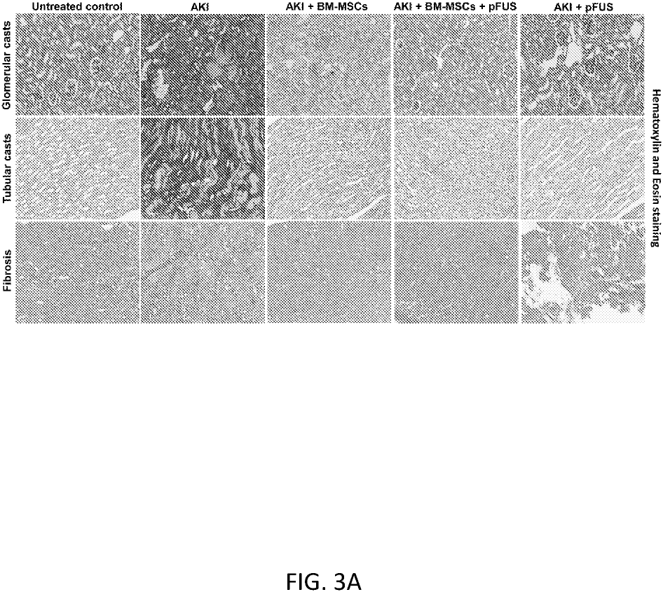

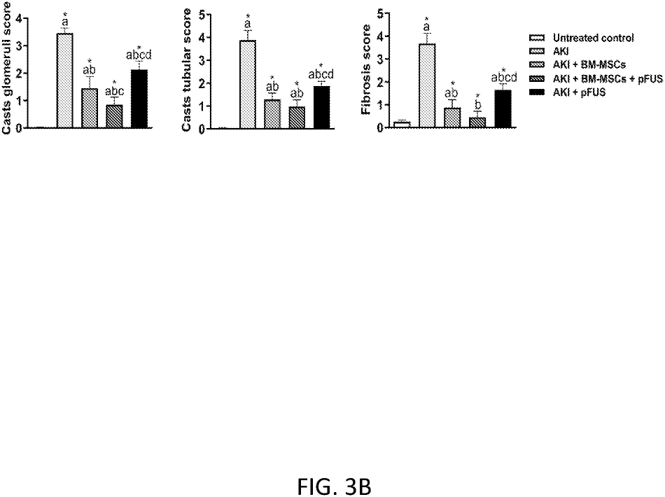

Histological evaluation of kidney tissue demonstrated that cisplatin induced a significant increase in indices of kidney injury, such as glomerular casts, tubular casts, and fibrosis, compared to untreated controls (FIGS. 3A, 3B). When AKI animals were treated with BM-MSCs alone, there was a significant reduction in all these indices compared to AKI group (Tubular Casts:
1.29±0.28 vs. 3.88±0.43, p<0.05; Glomerular Casts: 1.45±0.42 vs. 3.46±0.18, p<0.05; Fibrosis Score: 0.88±0.34 vs. 3.68±0.44, p<0.05). Treatment with pFUS alone also yielded some improvement compared to AKI group (Tubular Casts: 1.89±0.20 vs. 3.88±0.43, p<0.05;
Glomerular Casts: 2.13±0.31 vs. 3.46±0.18, p<0.05; Fibrosis Score: 1.65±0.28 vs. 3.68±0.44, p<0.05). However, animals in the combined pFUS + BM-MSC treatment group showed less histological evidence of kidney damage than those in BM-MSCs alone group (Tubular Casts:
0.98±0.28 vs. 1.29±0.28, p>0.05; Glomerular Casts: 0.85±0.28 vs. 1.45±0.42, p<0.05; Fibrosis Score: 0.46±0.26 vs. 0.88±0.34, p>0.05). (FIGS. 3A, 3B).

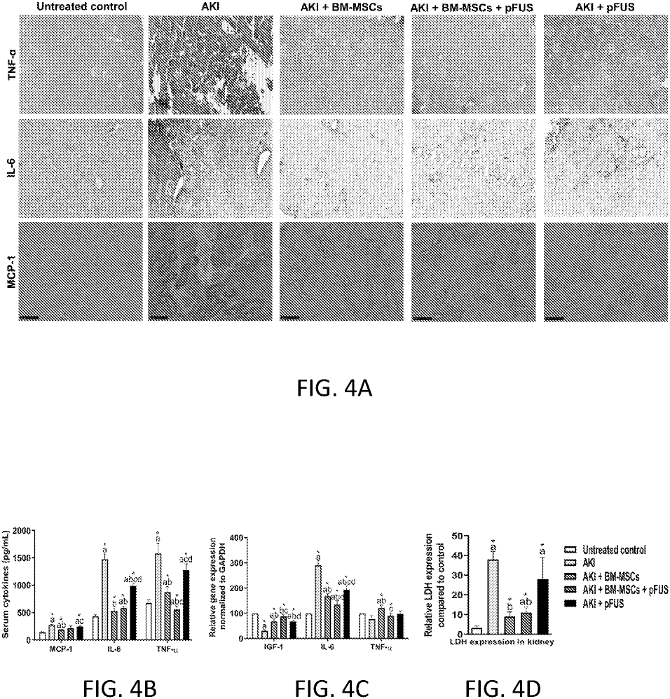

Immunohistochemical evaluation of kidney tissue demonstrated that AKI increased the expression of inflammation markers, including tumor necrosis factor-alpha (TNF-α), interleukin-6 (IL-6) and monocyte chemoattractant protein-1 (MCP-1) (FIG. 4A). While treatment of animals with either BM-MSCs alone or pFUS alone reduced the amount of staining of all these markers compared to AKI group, the most significant reduction was seen in the group of animals which received treatment with both pFUS + BM-MSCs (FIG. 4A).

Similar results were seen when examining the serum levels of these inflammatory cytokines. Our data showed that serum cytokines were significantly increased in animals with AKI compared to untreated controls, both for TNF-α (1,579±189 vs. 676±59 pg/mL, p<0.05) and IL-6 (1,477±92 vs. 433±26 pg/mL, p< 0.05) levels. This effect was attenuated when animals with AKI were treated with BM-MSCs alone (TNF-α: 873±100 pg/mL, p<0.05 and IL-6: 534±39 pg/mL; p<0.05; (FIG. 4B), or with both pFUS + BM-MSCs (TNF-α: 558±189 pg/mL, p<0.05 and IL-6: 587±28 pg/mL, p<0.05; FIG. 4B); in fact, the combined treatment of BM-MSCs and pFUS resulted in a greater suppression of TNF-α than did either treatment alone. There was no significant change in serum MCP-1 levels across all groups. Both TNF-α and IL-6 demonstrated a similar pattern when their gene expression was assessed at the RNA level by qRT-PCR (FIG. 4C). Lactate dehydrogenase (LDH), a marker for renal damage, was significantly upregulated in the kidney for animals with AKI, compared to untreated controls (38.69±3.99 vs. 3.30±1.03 relative units, p<0.05). pFUS alone did not significantly reduce LDH levels compared to the AKI group (28.00±11.00 vs. 38.69±3.99 relative units, p>0.05). However, LDH levels were significantly reduced by both BM-MSCs alone (38.69±3.99 vs. 9.00±2.35 relative units, p<0.05) and the combined pFUS + BM-MSCs treatment (38.69±3.99 vs. 10.70±2.68 relative units, p<0.05) (FIG. 4D).

### The Molecular Effect of BM-MSCs and pFUS on the Kidney in the Setting of AKI

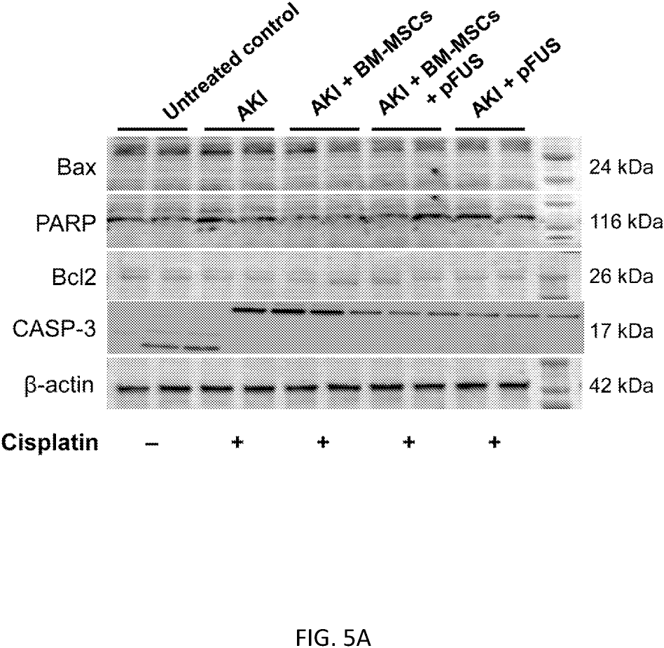

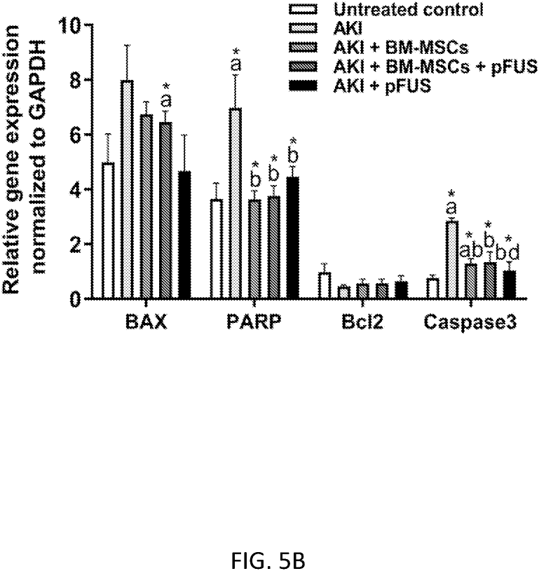

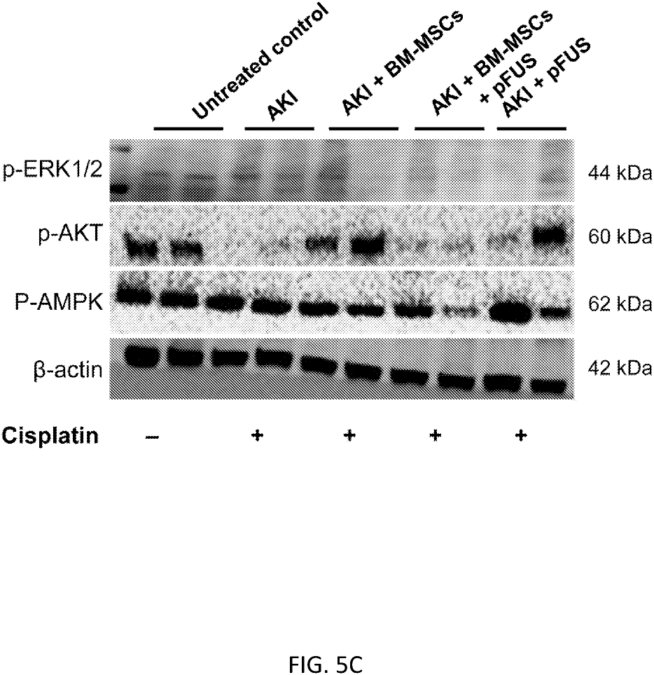

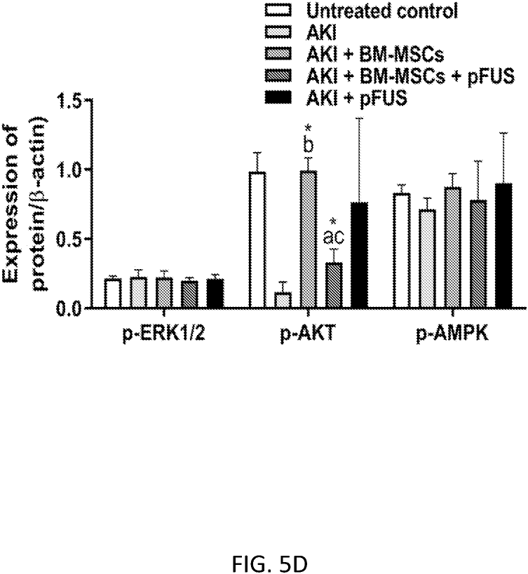

We measured the expression of pro-apoptotic and anti-apoptotic proteins, including Bax (pro-apoptotic), Bcl-2 (anti-apoptotic), caspase-3 (an executioner caspase), and PARP (contributes to both apoptosis and DNA repair) in all collected kidney samples. Western blot analysis revealed cleaved caspase-3 bands in all mice which suffered AKI following cisplatin administration, thereby indicating apoptotic activity (FIG. 5A). RT-PCR levels normalized to GADPH found that Bcl-2 activity was low across all treatment groups (FIG. 5B). However, the expression of Bax, PARP and caspase-3 were all elevated in animals in the AKI group compared to the untreated control group (Bax: 7.99±1.37 vs. 4.99±1.04, p>0.05; PARP: 6.98±1.20 vs. 3.65±0.59, p<0.05; Bcl2: 0.45±0.06 vs. 0.98±0.31, p>0.05; Caspase-3: 2.85±0.10 vs. 0.77±1.11, p<0.05). Treatment with BM-MSCs alone, pFUS alone, and pFUS + MSCs were all able to reduce the expression of these pro-apoptotic genes, often to levels comparable to untreated controls.

We then measured the protein expression of p-ERK1/2 (extracellular signal-regulated kinase ½), p-AKT (phosphorylated/activated Akt) and p-AMPK (phosphorylated/activated adenosine 5' monophosphate-activated protein kinase) in kidney lysates (FIGS. 5C and 5D). Levels of p-ERK1/2 and p-AMPK were unchanged among all treatment groups. We found that p-AKT was reduced in the AKI group, compared to untreated controls (0.12±0.07 vs 0.99±0.14 arbitrary units, p>0.05). In animals with AKI, treatment with either BM-MSCs alone or pFUS alone was able to restore p-AKT levels to levels comparable to untreated controls (0.99±0.09 arbitrary units, p<0.05; 0.77±0.60 arbitrary units, p<0.05, respectively). The combined pFUS + BM-MSC treatment was also able to increase p-AKT levels compared to AKI, though the change was not statistically significant (0.33±0.09 relative units, p>0.05).

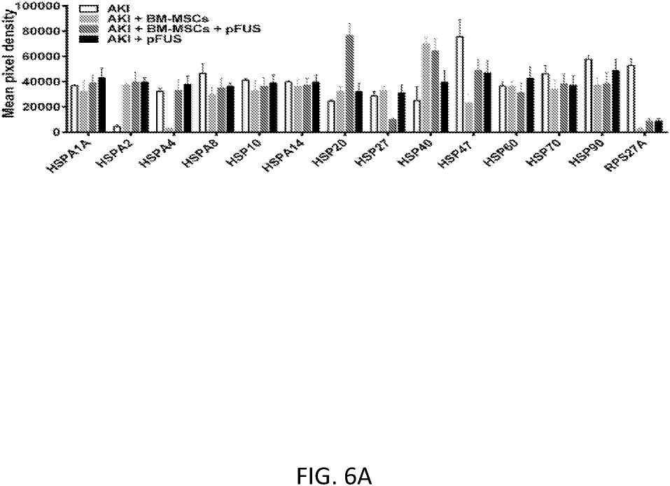

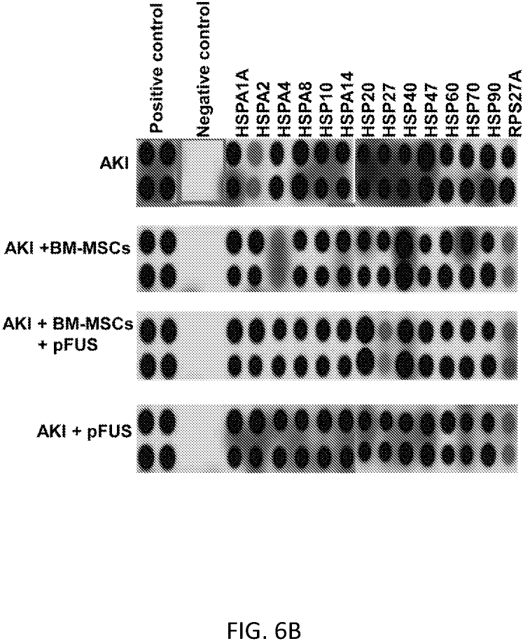

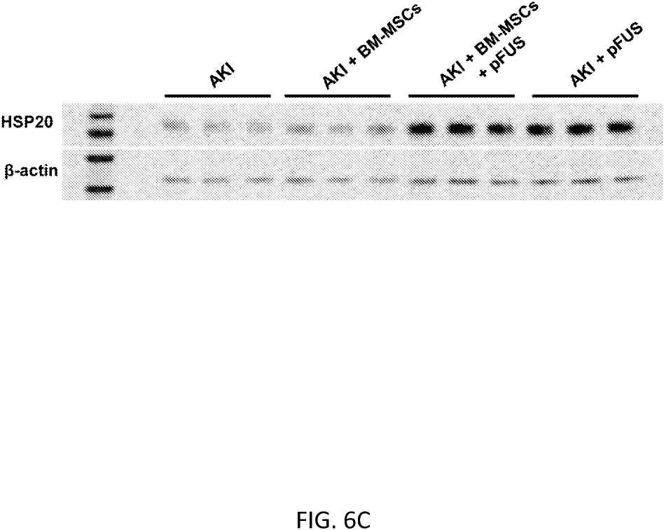

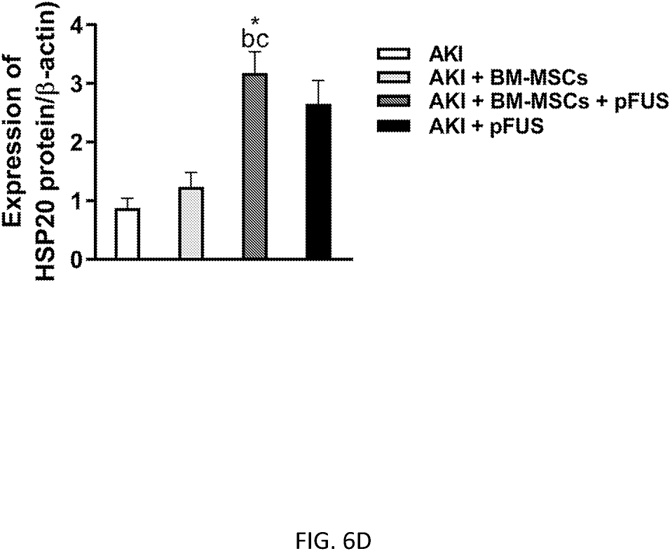

Next, we evaluated the protein expression of heat shock proteins (HSPs) in all collected kidney samples. In the initial protein array screen, HSP20 and HSP40 were found to be significantly elevated in mice treated with BM-MSCs, compared to the AKI group (FIGS. 6A, 6B). While the level of HSP20 increased only moderately in mice treated with BM-MSCs alone compared to AKI group (from 24,567±987 to 32,097±4,098 mean pixel density units), this value was greatly increased in those animals treated with both pFUS + BM-MSCs (76,542±9,765 mean pixel density units). For HSP40, the value was significantly increased relative to AKI animals in both the BM-MSCs alone and pFUS + BM-MSCs groups (from 25,098±10,876 to 69,872±5,672 and 64,351±9,876 mean pixel density units, respectively). To validate this significant increase in HSP20 in the AKI animals treated with BM-MSCs and pFUS, we performed western blot analysis and quantification which confirmed the same pattern (FIG. 6C).

### The Interaction Between HSP20 and HSP40 and Embryonic Kidney Cells

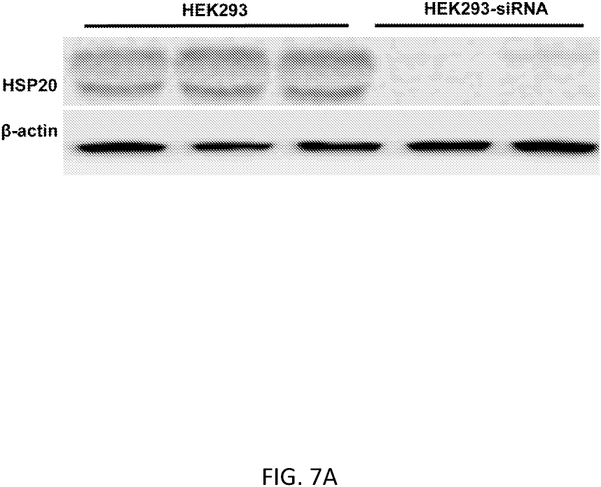

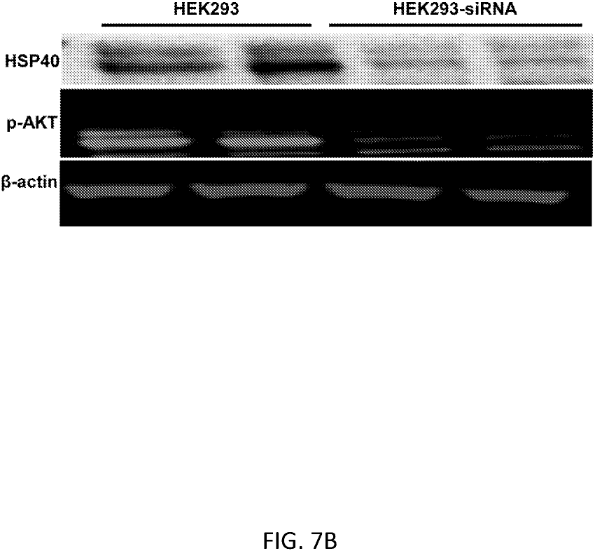

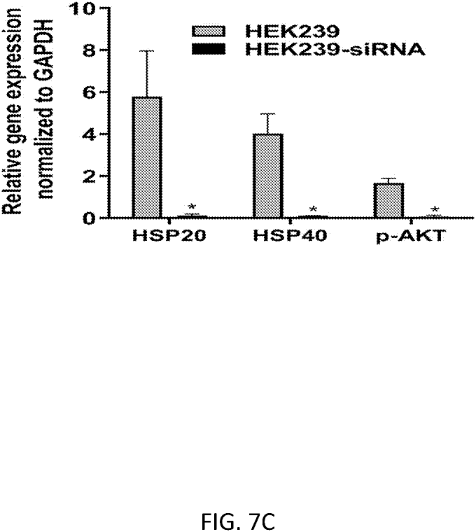

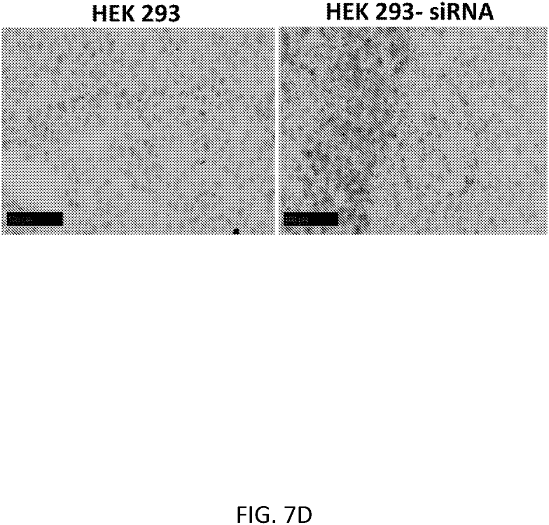

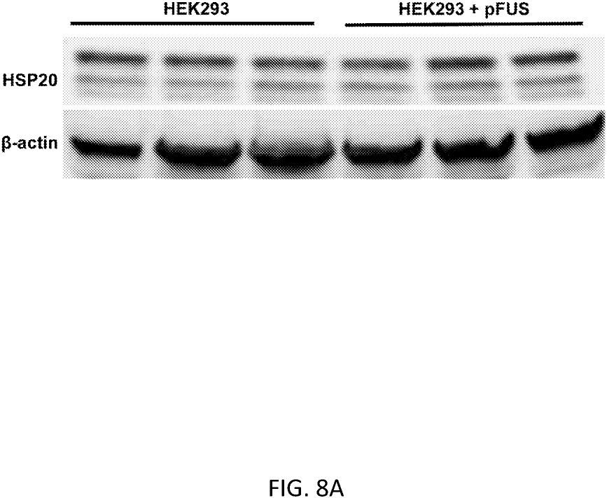

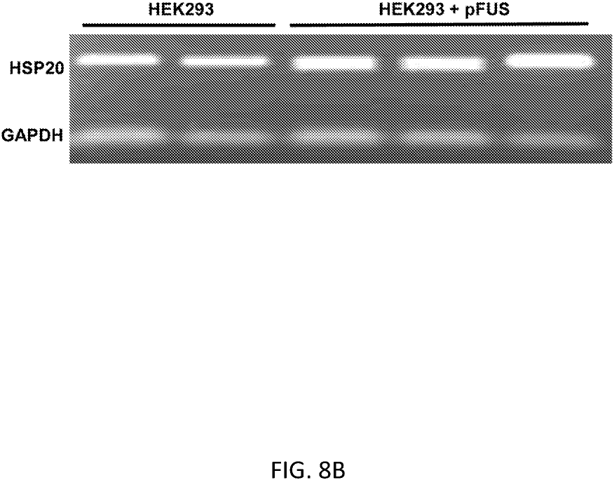

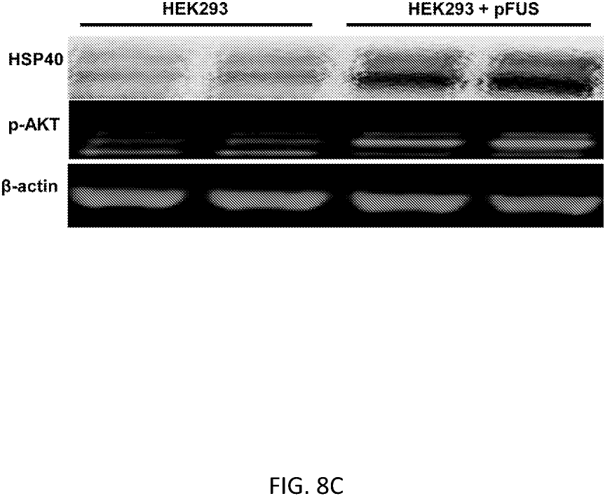

To study the relevance of HSP20 and HSP40, we constructed an siRNA to knock down HSP20 in human embryonic kidney cells (HEK293). Successful knockdown of HSP20 was confirmed by Western blot (FIG. 7A). HSP20 knockdown also resulted in a reduction in expression of HSP40 and p-AKT levels (FIGS. 7A, 7B). These results were further confirmed at the mRNA level via qRT-PCR analysis, which demonstrated a significant decrease in gene expression between HEK293 and HEK293-siRNA groups (HSP20: 5.79±2.19 vs. 0.12±0.08, p<0.05; HSP40: 4.03±0.93 vs. 0.11 ±0.01, p<0.05; p-AKT: 1.68±0.20 vs. 0.08±0.06, p<0.05) (FIG. 7C). Finally, HSP20 knockdown induced cell senescence, as measured by beta galactosidase staining (FIG. 7D). When HEK293 cells were treated with pFUS, they expressed increased levels of HSP20 and HSP40, as confirmed by Western Blot and qRT-PCR (FIGS. 8A, 8B). This also resulted in an increase in expression in p-AKT levels (FIG. 8C).

### Discussion

In this study, we have demonstrated that BM-MSCs and pFUS play a role in ameliorating the damage caused by cisplatin-induced AKI. This regeneration was mediated by a reduction of inflammatory cytokines, decreased apoptosis, and increased cell survival and proliferation in the kidney tissue. These effects were shown to be linked to upregulation of heat shock proteins HSP20 and HSP40, and subsequent activation of PI3K/Akt signaling, a classical signaling pathway for cell survival and growth. Although combined pFUS + BM-MSC treatment was not consistently superior to the individual treatments in animals with AKI, there were some areas where there was a suggestion of a synergistic effect. For instance, our histological data showed that combined pFUS + BM-MSCs yielded less kidney injury, demonstrated by glomerular casts, tubular casts, and fibrosis, compared to either BM-MSCs alone or pFUS alone. Given the short time course of this study (15 days), this histological evidence may be a more important prognostic factor for long-term kidney function than transient inflammatory or apoptosis markers.

Several results in our study differ from previous reports, particularly in three areas: (1) the generation of a cytokine gradient following pFUS, (2) the effect of pFUS on BM-MSC homing, and (3) the therapeutic effect of pFUS alone in the setting of AKI. Ziadloo et al. report that sonication of kidney tissue with pFUS induces a transient increase in cytokines, growth factors, and cell adhesion molecules, lasting for at least 3 days.²⁵ This local gradient is believed to serve as a beacon to increase BM-MSC homing. Similar to our study, Burks et al. tested the effect of pFUS on MSC homing in the context of cisplatin-induced AKI.^{26,27} They report that pFUS increased MSC homing around 1.4-fold, and significantly improved renal function and regeneration compared to MSC treatment alone. Furthermore, they reported that pFUS alone had no effect on renal function, apoptosis, or proliferation. However, we observed neither the transient cytokine gradient 2 days post-sonication, nor the resulting increase in MSC homing. Instead, we did observe that pFUS alone has a beneficial therapeutic effect, reducing histological evidence of injury, attenuating inflammation and apoptosis, and promoting proliferation through PI3K/Akt signaling. These discrepancies are probably best explained by differences in sonication parameters; in our study, although we used the same duty cycle, we used a lower pressure (i.e., acoustic energy) of 3 MPa compared to that previously reported at 8.9 MPa. Clearly, different sonication parameters will result in different biological effects,²⁹ and in the present study the sonication parameters used appears to upregulate heat shock proteins rather than cytokines and growth factors that facilitate BM-MSC homing. More direct comparisons between our studies and previous literature are made difficult by discrepancies in methodological reporting, making exact replication of sonication parameters difficult.

Instead, our findings suggest that these protective effects may be exerted via protein intermediaries. Using western blot and real-time PCR analysis, we found that BM-MSCs and pFUS, both alone and in combination, result in a significant upregulation of heat shock proteins HSP20 and HSP40, as well as activation of PI3K/Akt signaling. In general, heat shock proteins are upregulated in response to stress conditions, whether environmental, metabolic or pathophysiological.³⁰ HSP20 (also called HSPB6) is a member of the heat shock protein family called small heat shock proteins which, acting as ATP-independent molecular chaperones, inhibit or modulate protein aggregation and improve disaggregation processes.³¹ Working in conjunction with other heat shock protein families, they thus provide protection against the protein aggregates that could interfere with cellular processes and result in depletion of the factors that are required for protein homeostasis.^{32,33} Members of the small heat shock protein family are known to have anti-apoptotic, anti-inflammatory, and pro-angiogenic properties.³⁰ HSP20 is known to be expressed in kidney tissue, as well as in the liver, brain, lung, stomach, blood, smooth muscle, skeletal muscle, and cardiac tissue.³⁴ In addition to kidney injury, increased HSP20 levels are associated with cell protection in MSC transplantation,³⁵ cardiac ischemia/reperfusion injury,³⁶ doxorubicin-induced cardiomyopathy,³⁷ and sepsis-triggered myocardial dysfunction.³⁸ In the present study, we hypothesize that the increase in HSP20 may play a role in kidney tissue regeneration and repair which we observed. It is also possible that the increase in HSP40 plays an important role. HSP40 is a downstream target of HSP20 which is known to deliver unfolded or newly-synthesized amino acid chains to HSP70 which, in turn, assists them in folding into their proper functional forms.³⁹

Upregulation of p-AKT is likely another important component of pFUS and MSC therapy, since it is known to be involved in the regeneration and repair processes of damaged kidney tissue.^{40,41} PI3K/AKT signaling is a classical pathway for cell proliferation and survival,⁴² and there is evidence that it is activated by HSPs, as our knockdown experiments have confirmed. Rat MSCs genetically engineered to overexpress HSP20 demonstrated better survival in response to oxidative stress both in vitro and in vivo, and was better able to improve cardiac function in a rat model of myocardial infarction compared to control MSCs; the beneficial effects of HSP20 overexpression were associated with increased Akt phosphorylation and increased secretion of growth factors.³⁵ HSP20 transgenic mice are also more resistant to doxorubicin-induced cell death, with improved cardiac function and prolonged survival after chronic doxorubicin administration; again, the beneficial effects of HSP20 overexpression appeared to be dependent on direct interaction with p-AKT.³⁷ Here, we verified the same associations between HSP20, HSP40 and p-AKT in vitro that we observed in vivo. Given our results, we propose a model of AKI repair whereby HSP20/40 upregulation activates p-AKT, thus regulating anti-apoptotic activity through caspase-3 (considered the most important of the executioner caspases), Bcl-2 (an anti-apoptotic protein), Bax (a pro-apoptotic protein) and PARP (poly [ADP-ribose] polymerase) which plays a dual role in both DNA repair and apoptosis.⁴³ Activation of caspase-3 and induction of PARP, Bcl-2 and Bax are crucial mediators of programmed cell death.

Another possible repair mechanism is that BM-MSCs counter inflammation by reducing multiple inflammatory cytokines. The significant increases in serum TNF-α and IL-6 that we observed in AKI animals compared to untreated controls were substantially suppressed in animals treated with BM-MSCs alone, pFUS alone, or both. Indeed, the anti-inflammatory effects of BM-MSCs is believed to be a major mechanism by which they protect against renal damage.⁴⁴

Several factors will influence the clinical adoption of BM-MSCs and pFUS therapy for treating AKI. On a positive note, there are already Food and Drug Administration (FDA)-approved uses of image-guided therapeutic ultrasound in several areas of medicine, including in the treatment of cardiovascular diseases, tumor ablation, and neurosurgery.⁴⁵ In addition, therapeutic ultrasound of the kidneys could be easily carried out at the bedside with the right equipment, easing the adoption of such technologies.⁴⁶ Other researchers have noted that there are also pharmacological approaches to increasing HSP20 that might be considered.⁴⁷ For example, both sildenafil and vasoactive intestinal peptide (VIP) have been shown to induce substantial HSP20 phosphorylation in animal models.^{48,49} However, these medicinal approaches lack the precision and local targeting made possible by therapeutic ultrasound. Systemic activation of HSP20 and thus PI3K/AKT signaling may carry the risk of tumorigenesis; indeed, inappropriate PI3K/AKT signaling is associated with many cancers, and there is ongoing research aimed at developing inhibitors of AKT and its downstream target mTOR.⁴² Presuming that additional work confirms our findings, further research would be required to confirm that the positive regenerative effects of HSP20/p-AKT upregulation do not come at the cost of significant safety concerns.

One limitation of this study is that there are many types of cell types within the kidney, and it is unknown which are the exact targets of BM-MSC-mediated regeneration. Our in vitro studies on the interaction between HSP20/40 and p-AKT used human embryonic kidney (HEK), but the lineal identity of this cell line is poorly-defined.⁵⁰ Further in vivo studies involving HSP inhibitors and knockout mouse models are thus needed to understand the molecular mechanism through which HSP20 might improve kidney tissue repair, and to assess more fully how pFUS and BM-MSCs interact. Thus, while we cannot currently make the strong claim that HSP20/40 is directly mediating the therapeutic effect of pFUS and MSCs, our study is the first to link the two, prompting further experiments to elucidate the exact molecular mechanisms. It will also be important to optimize sonication parameters; discrepancies between our studies and previous ones clearly demonstrate that varying the sonication intensity changes the resulting bioeffects. Understanding the relationship between sonication parameters and bioeffects will be essential for determining the best outcomes for clinical translation.

In conclusion, we have shown that both pFUS and BM-MSCs independently result in improved kidney function and regeneration in a mouse model of cisplatin-induced AKI. The beneficial effects appear to be associated with upregulation of HSP20 and p-AKT, reduction of inflammatory cytokines, and increased secretion of growth/regeneration-promoting factors.

### Material and Methods

### Mice and Animal Models

Eight-week-old female wild-type CD1 mice (34±1.22 g total body weight), were acquired from Charles River Laboratories (Wilmington, Massachusetts, USA) and maintained under standard conditions in a pathogen-free facility. Food and water were available to mice throughout the study. Experiments began 2 weeks after animal arrival and all in vivo procedures were carried out according to approved guidelines of the institution Administrative Panel on Laboratory Animal Care (APLAC). To induce AKI, cisplatin (Sigma Aldrich Corporation, St. Louis, Missouri, USA) was dissolved in saline and further diluted in phosphate buffer saline (PBS); this cisplatin mixture (15 mg/kg) was then injected intraperitoneally into mice via a single dose.

To investigate the protective effect of BM-MSCs and pFUS in cisplatin-induced AKI mice, BM-MSCs and pFUS were used either alone or in combination (FIG. 9A). Five groups were used in this study: Group 1: healthy untreated controls; Group 2: AKI, which received no treatment; Group 3: AKI and BM-MSCs alone (IV injection of 1×10⁶ BM-MSCs via a tail vein injection); Group 4: AKI and pFUS + BM-MSCs, which received pFUS 4 h before 1×10⁶ BM-MSCs IV; Group 5: AKI and pFUS alone. All treatments were given on day 3 post-cisplatin injection and mice were then observed for another 12 days. At the end of the experimental protocol (day 15), mice were humanely euthanized and their blood, urine and kidneys collected.

### Pulsed Focused Ultrasound (pFUS)

A 1.1 MHz central frequency custom focused ultrasound therapy transducer (H-102NRE, Sonic Concepts, Bothell, Washington, USA) with a 49 mm central opening was used for this study. The ultrasound transducer was then calibrated in a water tank filled with degassed and deionized water. The transducer was driven by an Agilent 33250A function generator (Agilent Technologies, Santa Clara, California, USA) and connected to a 50 dB ENI 525LA linear power amplifier (ENI Technology, Inc., Rochester, New York, USA) and an impedance matching circuit (Sonic Concepts, Bothell, Washington, USA). The transducer was excited at a central 1.1 MHz frequency with 20 cycles at 100 Hz pulse repetition frequency (PRF) in a “burst” mode. A hydrophone (HNR-0500, Onda Corporation, Sunnyvale, California, USA) was placed in the focal spot of the transducer (55 mm away from its surface) and an Acoustic Intensity Measurement System (AIMS III, Onda Corporation, Sunnyvale, CA, USA) was used for precise movement and positioning of the hydrophone and digitizing the waveforms. The measured beam profile (full width half-maximum area for pressure) at the focal area was 10 mm in length and 1.5 mm in diameter. The intensity and pressure measurements were performed for negative peak pressures (NPP) up to 3 MPa in order to reduce risks of hydrophone damage. The transducer was used with the following parameters: 5% duty cycle, 5 Hz pulse repetition frequency (PRF), 2.9 MPa NPP, and 272 W/cm² spatial average pulse average intensity (Iₛₐₚₐ).

For image-guided kidney therapy, a setup of co-aligned transducers was used. The imaging transducer (Siemens Acuson S2000 14L5 sp, Siemens Corporation, Washington, D.C., USA) was placed in the central opening of the focused ultrasound transducer. Both transducers were aligned and fixed with a custom-made 3D-printed holder. The focused ultrasound transducer’s focal spot was fixed at 55 mm axial and 0 mm lateral distance from the central point of the imaging transducer. Any misalignment of the focused ultrasound and imaging beam was checked several times in the water tank with the hydrophone and oscilloscope by assembling and disassembling the 3D-printed holder. The measured beam misalignment was less than 200 µm. The ultrasound guidance of the in vivo kidney therapy was done using a Siemens S2000 scanner (Siemens Medical Solutions, Issaquah, Washington, USA). Mice were kept under anesthesia and submerged vertically in the water tank with their head kept above the water surface. The assembled holder with the focused ultrasound and imaging transducers was then connected to a translation stage and kept in the water at approximately 50 mm axial distance from the mouse. The mouse’s kidney was identified on the Siemens S2000 scanner and placed at the desired location, 55 mm axially, 0 mm laterally from the central point of the imaging transducer. To make treatment uniform 8 non-overlapping regions in the kidneys were selected and treated for 30 seconds each, with a total pFUS therapy time of 4 minutes for each mouse kidney. The holder was moved from one spot to another using the translation stage so the distance between 2 adjacently treated spots was 2-3 mm.

### Cell Culture of the Bone Marrow-Derived Mesenchymal Stem Cells

Human BM-MSCs were isolated from 3 donors, labeled with green florescence protein (GFP, ATCC, USA), grown in culture and characterized. A frozen vial (1×10⁶ MSCs) was thawed at 37° C. and plated in complete culture medium consisting of α-minimum essential medium (α-MEM; Gibco, USA), 20% (vol/vol) FBS (Atlanta Biologicals, USA), 100 units/mL penicillin (Gibco, USA), 100 µg/mL streptomycin (Gibco, USA), and 2 mM I-glutamine (Gibco, USA). After 7 days, the cell layer was washed with PBS and the adherent viable cells were harvested using 0.25% trypsin and 1 mM EDTA (Gibco, USA) for 5 min at 37° C., reseeded at 1000 cells per cm² in culture medium, and incubated for 7 days until they reached 70-80% confluence. Cells from passage number 3 were used for all experiments.

### Measurements of Creatinine and Blood Urea Nitrogen

Blood samples were collected every 3 days following treatment and at sacrifice. Blood was collected in heparinized tubes and centrifuged at 14,000 g for 10 min to obtain plasma samples

Creatinine concentrations were measured using an enzyme-linked immunosorbent assay (ELISA; Stanbio, TX, USA). Blood urea nitrogen (BUN) concentrations were measured using a QuantiChrom Urea Assay Kit (DIUR-500, BioAssay Systems, Hayward, California, USA).

### Histology and Immunohistochemistry

All harvested kidneys were perfused with 4% (vol/vol) paraformaldehyde in PBS, fixed in formalin for 24 hr and then embedded in paraffin. Samples were then sectioned in 6 µm thick slices for hematoxylin & eosin and immunohistochemistry. To determine the pathological score, hematoxylin & eosin-stained preparations were evaluated under a light microscope. Dilated tubules, tubular casts, necrosis and tubular degeneration were scored as described previously.⁵¹ In kidney images, glomeruli and tubular casts and fibrosis were assessed in non-overlapping fields (up to 20 for each section) using 40 objective images, in a single-blind fashion. Glomerular casts and tubular casts were assessed by calculating the percentage of the corresponding structure positive for cast formation. Fibrosis was quantified by calculating the percentage of glomeruli showing evidence of fibrosis. The scoring system used is described as follows. Kidneys showing no injury were marked 0. Kidneys exhibiting minimal (<10%), mild (10-25%), moderate (26-50%), extensive (51-75%) and severe (>75%) injuries were assigned scores of 1, 2, 3, 4 and 5, respectively.

For immunohistochemical staining, paraffin-embedded kidney sections were deparaffinized, hydrated, and antigen-retrieved; endogenous peroxidase activity was quenched by 3% H₂O₂. Sections were then blocked with 10% normal donkey serum, followed by incubation overnight at 4° C. with antibodies including anti-MCP-1, IL-6, and TNF-α. Next, samples were incubated with secondary antibody for 1 hour, and then incubated with DAB (3,3-diaminobenzidine) (Vector Laboratories, Burlingame, California USA). Slides were viewed with a Nikon Eclipse 80i microscope equipped with a digital camera (Nikon, Melville, New York, USA).

### Flow-Cytometric Analysis

In brief, kidney tissues were cut into small pieces and passed through a 70 µm cell strainer to remove any large pieces. The cells clusters were digested with collagenase dispase (Sigma Aldrich, St. Louis, Missouri, USA) at 37° C. for 30 min and then dissociated cells filtered (30 µm). The cells were then stained at 4° C. in a solution of PBS containing 2% fetal bovine serum. GFP fluorescence was examined using the FITC channel (excitation by blue laser at 488 nm and detection through a 525/550 nm filter). One million cells in 100 µL were incubated for 30 minutes in the presence of the antibody lactate dehydrogenase (LDH; Cell Signaling, CA, USA) under saturating conditions (5 µL), and then washed. Analysis was performed using the FACS analysis De Novo software (Glendale, CA, USA).

### Cell Culture

Human embryonic kidney 293 cells (HEK 293; ATCC, USA) were seeded on 100 cm² culture dishes to 60-70% confluence in complete medium containing 20% FBS for 3 days (culture medium was same as described above for BM-MSCs). For siRNA knockdown of HSP20, the cells were washed twice with PBS and then siRNA specific for HSP20 knockdown (OriGene, USA) was added (4 nM) for 24 hours. The cells were then harvested and analyzed by western blot and real-time PCR.

### Western Blot Analysis

Kidney lysate, cultured BM-MSCs and HEK cells were lysed in 1 × SDS sample buffer. The kidneys were lysed with RIPA solution containing 1% NP40, 0.1%SDS, 100 mg/ml PMSF, 1% protease inhibitor cocktail, and 1% phosphatase I and II inhibitor cocktail (Sigma, St Louis, Missouri, USA) on ice. The supernatants were collected after centrifugation at 13,000 × g at 4° C. for 30 min. Protein concentration was determined by bicinchoninic acid protein assay (Sigma Aldrich, USA). An equal amount of protein was loaded into a 10% or 15% SDS-PAGE and transferred onto polyvinylidene difluoride membranes. The primary antibodies were as follows: anti-ERK1/2, pAKT, BAX, BCL2, HSP20, IL-6, MCP-1, and anti-AMPKα (Cell Signaling Technology, USA), anti-cleaved caspase3, and PARP (Santa Cruz, USA), anti-β-actin (cat: sc-1616, Santa Cruz Biotechnology, USA). Quantification was performed by measuring the intensity of the signals with the aid of the National Institutes of Health Image J software package (Image J, NIH, USA)

### Real Time PCR

Total RNA was extracted using an RNeasy Mini kit (QIAGEN, CA, USA) and used to synthesize first-strand cDNA by reverse transcription (Applied Biosystems, CA, USA). The quality of cDNA was assessed by the ratio of the absorbance at 260 and 280 nm. Real-time PCR for mouse IGF-1, 1L-6, TNF_{α}, BAX, PARP, BCL2, Caspase3, HSP20 HSP40 and GAPDH was performed using TaqMan Gene Expression Assays (Applied Biosystems) and TaqMan Fast Master Mix (Applied Biosystems). All PCR probe sets were purchased from Applied Biosystems. The assays were performed in triplicates for each biological sample. For data analysis, we adopted the 2-^{ΔΔCt} method.⁵² The threshold cycle (Ct) of each target gene was first normalized to the Ct of GAPDH in each sample and then to the corresponding value in the control sample.

### β-Galactosidase (SA-β-Gal) Assay for HEK 293 Cells

Cellular senescence was measured by β-galactosidase staining. For assessment of β-galactosidase activity (blue stain positive cells), the cells were fixed with 4% paraformaldehyde for 30 min, washed with PBS, and stained with a β-galactosidase detecting kit (Thermos Fisher, USA) according to the manufacturer’s instruction and examined under a phase contrast microscope.

### Enzyme-Linked Immunosorbent Assay

In brief, we measured the expression level of cytokines in serum obtained from animals belonging to different groups with an ELISA (ELISA; R&D Systems, CA, USA) using 96-well array plates that were pre-coated with specific cytokine capture antibodies which included (MCP-1), IL-6, and TNF-α, as previously described.⁵³ We then confirmed the concentration of these targets through quantitative PCR.

### Statistical Analysis

All data are presented as the mean ± standard deviation (SD). Statistical analysis of the data was performed using the Sigma Stat software 4.0 (Systat Software, San Jose, California, USA) and GraphPad Prism (GraphPad Software, Inc., San Diego, California, USA). Comparison among three or more groups was made using one-way or two-way ANOVA, followed by the Tukey’s test. Comparison between two groups was made using Student’s t-test. P< 0.05 was considered statistically significant.

### References

1 Levey, A. S. & James, M. T. Acute Kidney Injury. Ann Intern Med 167, ITC66-ITC80, doi:10.7326/AITC201711070 (2017).

2 Pavkov, M. E., Harding, J. L. & Burrows, N. R. Trends in hospitalizations for acute kidney injury - United States, 2000-2014. MMWR Morb Mortal Wkly Rep 67, 289-293, doi:10.15585/mmwr.mm6710a2 (2018).

3 Carlson, N. et al. Trends in one-year outcomes of dialysis-requiring acute kidney injury in denmark 2005-2012: a population-based nationwide study. PLoS One 11, e0159944, doi:10.1371/journal.pone.0159944 (2016).

4 Kolhe, N. V., Muirhead, A. W., Wilkes, S. R., Fluck, R. J. & Taal, M. W. National trends in acute kidney injury requiring dialysis in England between 1998 and 2013. Kidney Int88, 1161-1169, doi:10.1038/ki.2015.234 (2015).

5 Coca, S. G., Singanamala, S. & Parikh, C. R. Chronic kidney disease after acute kidney injury: a systematic review and meta-analysis. Kidney Int 81, 442-448, doi:10.1038/ki.2011.379 (2012).

6 Chawla, L. S. & Kimmel, P. L. Acute kidney injury and chronic kidney disease: an integrated clinical syndrome. Kidney Int 82, 516-524, doi:10.1038/ki.2012.208 (2012).

7 Chawla, L. S., Eggers, P. W., Star, R. A. & Kimmel, P. L. Acute kidney injury and chronic kidney disease as interconnected syndromes. N Engl J Med 371, 58-66, doi:10.1056/NEJMra1214243 (2014).

8 Pannu, N. Bidirectional relationships between acute kidney injury and chronic kidney disease. Curr Opin Nephrol Hypertens 22, 351-356, doi10.1097/MNH.0b013e32835fe5c5 (2013).

9 Coca, S. G., Cho, K. C. & Hsu, C. Y. Acute kidney injury in the elderly: predisposition to chronic kidney disease and vice versa. Nephron Clin Pract 119 Suppl 1, c19-24, doi:10.1159/000328023 (2011).

10 Hsu, R. K. & Hsu, C. Y. The Role of Acute Kidney Injury in Chronic Kidney Disease. Semin Nephrol36, 283-292, doi:10.1016/j.semnephrol.2016.05.005 (2016).

11 Chertow, G. M., Burdick, E., Honour, M., Bonventre, J. V. & Bates, D. W. Acute kidney injury, mortality, length of stay, and costs in hospitalized patients. J Am Soc Nephrol 16, 3365-3370, doi:10.1681/ASN.2004090740 (2005).

12 Leither, M. D. et al. The impact of outpatient acute kidney injury on mortality and chronic kidney disease: a retrospective cohort study. Nephrol Dial Transplant 34, 493-501, doi:10.1093/ndt/gfy036 (2019).

13 Liu, K. D. & Brakeman, P. R. Renal repair and recovery. Crit Care Med 36, S187-192, doi:10.1097/CCM.0b013e318168ca4a (2008).

14 Bruno, S., Chiabotto, G. & Camussi, G. Concise review: different mesenchymal stromal/stem cell populations reside in the adult kidney. Stem Cells Transl Med 3, 1451-1455, doi:10.5966/sctm.2014-0142 (2014).

15 Herrera, M. B. et al. Mesenchymal stem cells contribute to the renal repair of acute tubular epithelial injury. IntJ Mol Med 14, 1035-1041 (2004).

16 Morigi, M. & De Coppi, P. Cell therapy for kidney injury: different options and mechanisms--mesenchymal and amniotic fluid stem cells. Nephron Exp Nephrol 126, 59, doi:10.1159/000360667 (2014).

17 Caplan, A. I. Why are MSCs therapeutic? New data: new insight. J Pathol 217, 318-324, doi:10.1002/path.2469 (2009).

18 Schrepfer, S. et al. Stem cell transplantation: the lung barrier. Transplant Proc 39, 573-576, doi:10.1016/j.transproceed.2006.12.019 (2007).

19 Santeramo, I. et al. Human kidney-derived cells ameliorate acute kidney injury without engrafting into renal tissue. Stem Cells Transl Med 6, 1373-1384, doi:10.1002/sctm.16-0352 (2017).

20 Leibacher, J. & Henschler, R. Biodistribution, migration and homing of systemically applied mesenchymal stem/stromal cells. Stem Cell Res Ther 7, 7, doi:10.1186/s13287-015-0271-2 (2016).

21 Eggenhofer, E. et al. Mesenchymal stem cells are short-lived and do not migrate beyond the lungs after intravenous infusion. Front Immunol 3, 297, doi:10.3389/fimmu.2012.00297 (2012).

22 Fischer, U. M. et al. Pulmonary passage is a major obstacle for intravenous stem cell delivery: the pulmonary first-pass effect. Stem Cells Dev 18, 683-692, doi:10.1089/scd.2008.0253 (2009).

23 Burks, S. R. et al. Investigation of cellular and molecular responses to pulsed focused ultrasound in a mouse model. PLoS One 6, e24730, doi:10.1371/journal.pone.0024730 (2011).

24 Burks, S. R., Ziadloo, A., Kim, S. J., Nguyen, B. A. & Frank, J. A. Noninvasive pulsed focused ultrasound allows spatiotemporal control of targeted homing for multiple stem cell types in murine skeletal muscle and the magnitude of cell homing can be increased through repeated applications. Stem Cells 31, 2551-2560, doi:10.1002/stem.1495 (2013).

25 Ziadloo, A. et al. Enhanced homing permeability and retention of bone marrow stromal cells by noninvasive pulsed focused ultrasound. Stem Cells 30, 1216-1227, doi:10.1002/stem.1099 (2012).

26 Burks, S. R. et al. Pulsed focused ultrasound pretreatment improves mesenchymal stromal cell efficacy in preventing and rescuing established acute kidney injury in mice. Stem Cells 33, 1241-1253, doi:10.1002/stem.1965 (2015).

27 Burks, S. R. et al. Mesenchymal stromal cell potency to treat acute kidney injury increased by ultrasound-activated interferon-gamma/interleukin-10 axis. J Cell Mol Med22, 6015-6025, doi:10.1111/jcmm.13874 (2018).

28 Bi, B., Schmitt, R., Israilova, M., Nishio, H. & Cantley, L. G. Stromal cells protect against acute tubular injury via an endocrine effect. J Am Soc Nephrol 18, 2486-2496, doi:10.1681/ASN.2007020140 (2007).

29 Izadifar, Z., Babyn, P. & Chapman, D. Mechanical and biological effects of ultrasound: a review of present knowledge. Ultrasound Med Biol 43, 1085-1104, doi:10.1016/j.ultrasmedbio.2017.01.023 (2017).

30 Bakthisaran, R., Tangirala, R. & Rao Ch, M. Small heat shock proteins: Role in cellular functions and pathology. Biochim Biophys Acta 1854, 291-319, doi:10.1016/j.bbapap.2014.12.019 (2015).

31 Haslbeck, M., Weinkauf, S. & Buchner, J. Small heat shock proteins: Simplicity meets complexity. JBiol Chem 294, 2121-2132, doi:10.1074/jbc.REV118.002809 (2019).

32 Mogk, A., Bukau, B. & Kampinga, H. H. Cellular Handling of Protein Aggregates by Disaggregation Machines. Mol Cell 69, 214-226, doi:10.1016/j.molcel.2018.01.004 (2018).

33 Haslbeck, M. & Vierling, E. A first line of stress defense: small heat shock proteins and their function in protein homeostasis. J Mol Biol 427, 1537-1548, doi:10.1016/j.jmb.2015.02.002 (2015).

34 Fan, G. C., Chu, G. & Kranias, E. G. Hsp20 and its cardioprotection. Trends Cardiovasc Med 15, 138-141, doi:10.1016/j.tcm.2005.05.004 (2005).

35 Wang, X. et al. Hsp20-engineered mesenchymal stem cells are resistant to oxidative stress via enhanced activation of Akt and increased secretion of growth factors. Stem Cells 27, 3021-3031, doi:10.1002/stem.230 (2009).

36 Fan, G. C. et al. Novel cardioprotective role of a small heat-shock protein, Hsp20, against ischemia/reperfusion injury. Circulation 111, 1792-1799, doi:10.116/01.CIR.0000160851.41872.C6 (2005).

37 Fan, G. C. et al. Heat shock protein 20 interacting with phosphorylated Akt reduces doxorubicin-triggered oxidative stress and cardiotoxicity. Circ Res 103, 1270-1279, doi:10.1161/CIRCRESAHA.108.182832 (2008).

38 Wang, X. et al. Overexpression of Hsp20 prevents endotoxin-induced myocardial dysfunction and apoptosis via inhibition of NF-kappaB activation. J Mol Cell Cardiol 47, 382-390, doi:10.1016/j.yjmcc.2009.05.016 (2009).

39 Fan, C. Y., Lee, S. & Cyr, D. M. Mechanisms for regulation of Hsp70 function by Hsp40. Cell Stress Chaperones 8, 309-316, doi:10.1379/1466-1268(2003)008<0309:mfrohf>2.0.co;2 (2003).

40 Lan, A. & Du, J. Potential role of Akt signaling in chronic kidney disease. Nephrol Dial Transplant 30, 385-394, doi:10.1093/ndt/gfu196 (2015).

41 Zhang, G. et al. Protective effect of tempol on acute kidney injury through PI3K/Akt/Nrf2 signaling pathway. Kidney Blood Press Res 41, 129-138, doi:10.1 159/000443414 (2016).

42 Manning, B. D. & Toker, A. AKT/PKB Signaling: Navigating the Network. Cell 169, 381-405, doi:10.1016/j.cell.2017.04.001 (2017).

43 Elmore, S. Apoptosis: a review of programmed cell death. Toxicol Pathol35, 495-516, doi:10.1080/01926230701320337 (2007).

44 Morigi, M., Rota, C. & Remuzzi, G. Mesenchymal Stem Cells in Kidney Repair. Methods Mol Biol 1416, 89-107, doi:10.1007/978-1-4939-3584-0_5 (2016).

45 Cleary, K. & Peters, T. M. Image-guided interventions: technology review and clinical applications. Annu Rev Biomed Eng 12, 119-142, doi:10.1146/annurev-bioeng-070909-105249 (2010).

46 Chari, S., Nguyen, A. & Saxe, J. Stem cells in the clinic. Cell Stem Cell 22, 781-782, doi:10.1016/j.stem.2018.05.017 (2018).

47 Fan, G. C. & Kranias, E. G. Small heat shock protein 20 (HspB6) in cardiac hypertrophy and failure. J Mol Cell Cardiol 51, 574-577, doi:10.1016/j.yjmcc.2010.09.013 (2011).

48 Gilmont, R. R., Somara, S. & Bitar, K. N. VIP induces PKA-mediated rapid and sustained phosphorylation of HSP20. Biochem Biophys Res Commun 375, 552-556, doi:10.1016/j.bbrc.2008.08.050 (2008).

49 Tessier, D. J., Komalavilas, P., McLemore, E., Thresher, J. & Brophy, C. M. Sildenafil-induced vasorelaxation is associated with increases in the phosphorylation of the heat shock-related protein 20 (HSP20). J Surg Res 118, 21-25, doi:10.1016/j.jss.2004.01.001 (2004).

50 Lin, Y. C. et al. Genome dynamics of the human embryonic kidney 293 lineage in response to cell biology manipulations. Nat Commun 5, 4767, doi:10.1038/ncomms5767 (2014).

51 Han, S. J., Li, H., Kim, M., Shlomchik, M. J. & Lee, H. T. Kidney proximal tubular TLR9 exacerbates ischemic acute kidney injury. J Immunol 201, 1073-1085, doi:10.4049/jimmunol.1800211 (2018).

52 Ullah, M. & Sun, Z. Klotho deficiency accelerates stem cells aging by impairing telomerase activity. J Gerontol A Biol Sci Med Sci, doi:10.1093/gerona/gly261 (2018).

53 Amsen, D., de Visser, K. E. & Town, T. Approaches to determine expression of inflammatory cytokines. Methods Mol Biol 511, 107-142, doi:10.1007/978-1-59745-447-6_5 (2009).

### Example 2 A Novel Approach to Deliver Therapeutic Exosomes Directly into the Injured Mouse Kidney via Its Arterial Blood Supply

### Introduction

Mesenchymal stromal cells (MSCs) have shown promising results for kidney regeneration in the context of AKI and CKD [4]. The therapeutic effect of MSC-based therapies is thought to come from their ability to home to damaged tissue and secrete soluble factors with regenerative properties. Due to the paracrine nature of this mechanism, the proximity of infused MSCs to the injured site is critical for therapeutic efficacy [8, 9]. However, it is known that when MSCs are injected IV, the vast majority of cells become trapped in the pulmonary microvasculature, in what is known as the pulmonary first-past effect [10-14]. To avoid this intrinsic limitation, many groups have opted to instead study MSC-derived exosomes which are cell-free extracellular vesicles that carry a cargo of regenerative molecules [15]. Exosomes have been shown to avoid the pulmonary first-pass effect and have a therapeutic effect on par with the MSCs from which they are derived [16]. However, unlike MSCs there is little knowledge on how to further optimize delivery of exosomes to target organs [17]. Locoregional delivery, via the arterial blood supply, is perhaps one of the most straightforward and clinically translatable strategies for doing so.

Here, we develop a reproducible microsurgical technique to deliver therapeutics directly into each kidney in the mouse, via its arterial blood supply, while ensuring no operative morbidity or mortality. To demonstrate the therapeutic utility of this technique, we apply it to a mouse model of cisplatin-induced acute kidney injury (AKI), in which we perform IA injection of mesenchymal stromal cell (MSC)-derived exosomes.

### Materials and Methods

### Animal Protocols

All experimental procedures were performed in accordance with guidelines and regulations of the Administrative Panel on Laboratory Animal Care (APLAC) at Stanford University. A total of 50 healthy CD1 female mice (8 weeks old; body weight 28-31 g) were purchased from Charles River Laboratories (Wilmington, Massachusetts, USA) and housed at constant temperature, humidity and provided with normal feeding in 12:12 light-dark cycles. After 1 week of housing in separate cages in the adaptive environment, mice were randomly divided into 3 groups, group 1 had 10 mice and the other 2 groups had 20 mice each. Group 1 mice were designated as the untreated control group which received normal feeding while the other 2 groups were used to establish AKI models by intraperitonially injecting cisplatin (12 mg/kg) on day 0. To compare the protective effect of IA delivered exosomes in cisplatin-induced AKI, group 2 animals (internal control group) received IA injection of normal saline and group 3 animals received IA injection of exosomes (150 µg / 100 g body weight) on day 3. Mice were sacrificed at day 12 after cisplatin injection at which point blood, urine and kidney samples were collected. One kidney was immediately immersed in 10% neutral buffered formalin for histological analysis while the other kidney was immediately frozen in liquid nitrogen for biochemical marker measurement and western blot analysis

### Exosome Isolation, Characterization, and Purification

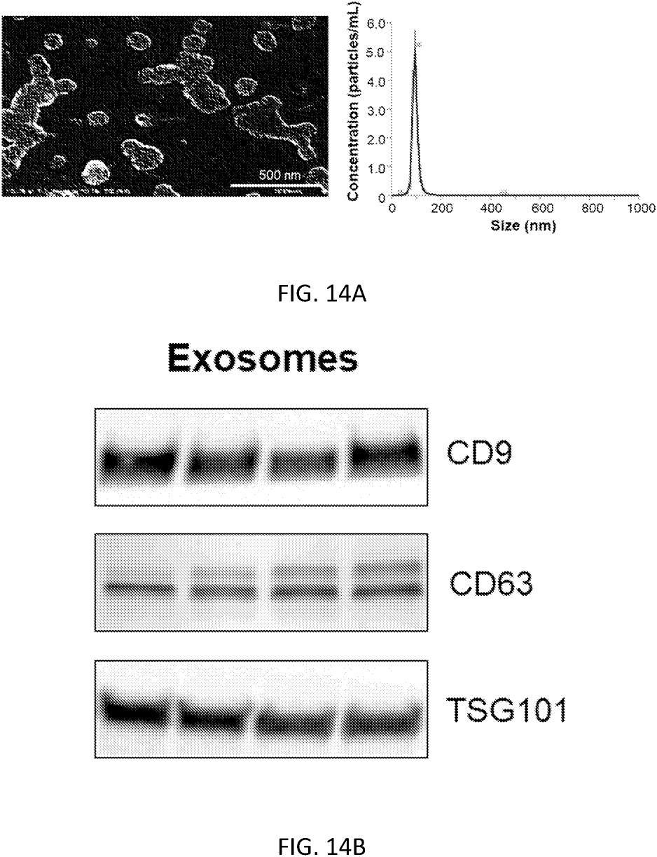

In our study we isolated and purified exosomes from cultured MSCs as previously described [18]. In brief, bone marrow-derived MSCs were cultured in a medium containing 20% fetal bovine serum (FBS), 100 U/mL penicillin and streptomycin (Thermo Fisher Scientific, USA), at room temperature with 5% CO₂ until passage 3. The passage 3 cells were cultured in serum free DMEM until they reached 80%-90% confluency overnight at room temperature, followed by centrifugation at 5,000 x g for 10 min at room temperature to remove cellular debris and the obtained supernatant was ultracentrifuged at 17,000 × g for 20 min and the exosomes were isolated from the supernatant obtained. An anion exchange resin (Q Sepharose Fast Flow, GE Healthcare, IL, USA) which was first balanced with 50 mM NaCl in 50 mM phosphate buffer and then washed with 100 mM NaCl in 50 mM phosphate buffer and later rinsed with 500 mM NaCl in 50 mM phosphate buffer was used to suspend conditioned medium. We used nanoparticle tracking analysis and transmission electron microscopy (TEM) to measure exosome size and number, which ranged from 20 to 180 nm with mean 118 nm and standard deviation 27 nm (FIG. 14A). Exosome surface markers CD9, CD63, and TSG101 were confirmed positive by Western blot (FIG. 14B).

### Intra-Arterial Injection Technique

Our technique for IA injection into the kidneys was optimized in an initial cohort of mice (n = 20) in which we established the vascular anatomy relevant to the kidneys and the optimal technique for therapeutic injection to both kidneys. This included examining different injection and suture sites as well as techniques to minimize dissection and operative time while ensuring adequate anatomical exposure. All surgeries were terminal and 100 µL of tattoo dye (1:10 dilution with normal saline) was used as a surrogate marker to determine the distribution of any therapeutic solution. For survival studies, we used our optimized technique and then administered 100 µL of normal saline, 50 µL to each kidney. Serum creatinine (SCr) and blood urea nitrogen (BUN) in mice at baseline and 24 hr after intra-arterial injection of saline were measured (Table 1).

Following isoflurane anesthesia, each animal was placed supine and its abdominal wall shaved. The animal was then prepped and draped in the usual sterile fashion and carefully opened with a midline incision, with the intestines carefully displaced cranially to expose each kidney. Both the inferior vena cava (IVC) and aorta were then visualized, with the latter covered by intra-abdominal adipose tissue.

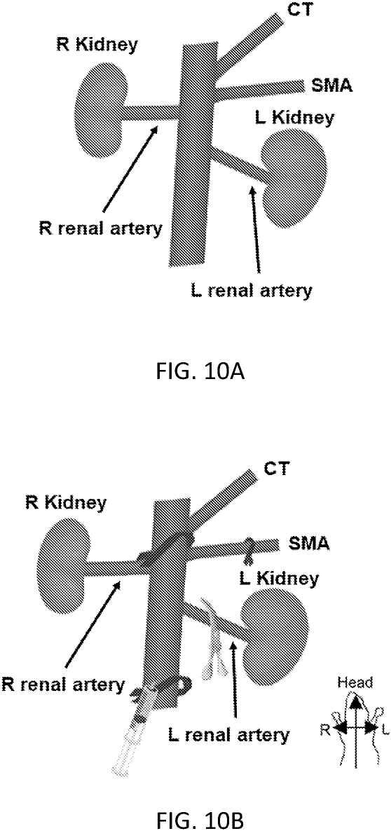

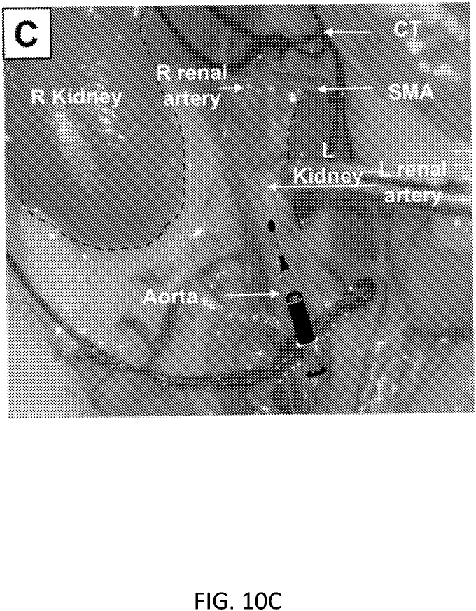

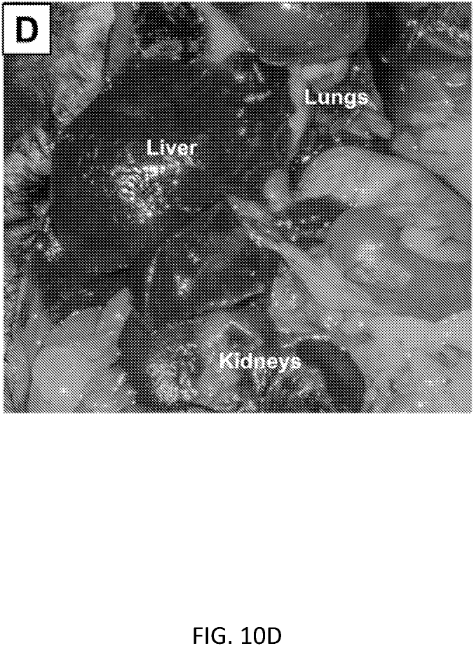

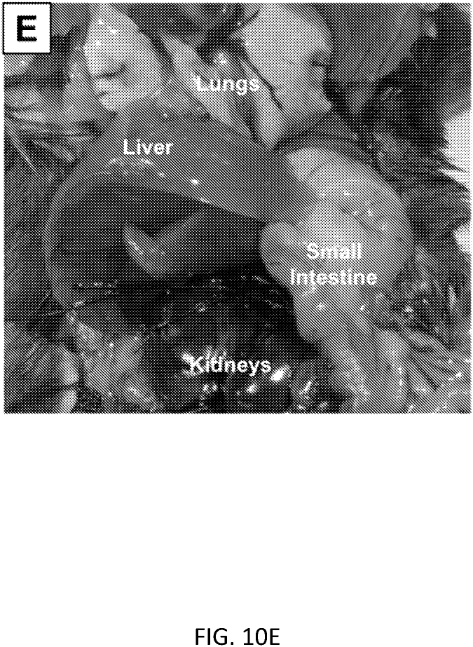

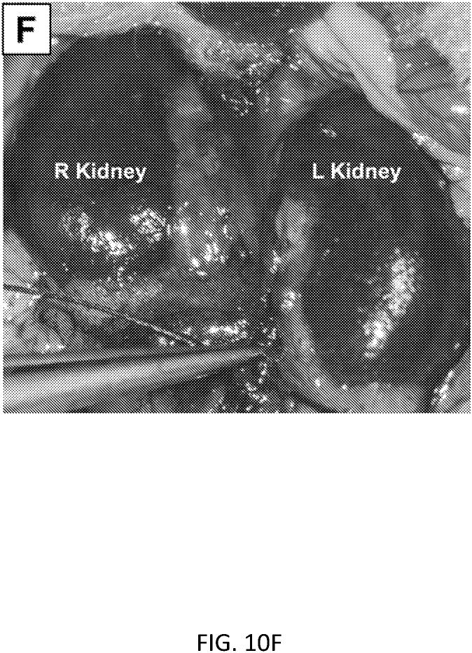

Arterial exposure: The adipose tissue surrounding the aorta was then carefully dissected to reveal the left renal artery, right renal artery, superior mesenteric artery (SMA) and celiac trunk (CT). We found that the safest plane of dissection is to start at the origin of the left renal artery (identified postero-inferiorly to the more prominent left renal vein) and then progress 1 cm superiorly along the length of the aorta to the origin of the right renal artery (which courses posteriorly around the IVC). This provides enough room for ligation of the aorta while also ensuring enough space to cannulate the aorta between the renal arteries. In our experience, we found that the location of the right renal artery is superior to the left renal artery; this is in contrast to humans where the right-sided liver positions the right renal artery slightly inferior to the left renal artery (FIG. 10A). Compared to the perpendicularly-angled origin of the right renal artery, the origin of the left renal artery is angled more caudally. Furthermore, we also found in the mouse that the SMA origin is located immediately superior to the right renal artery. Immediately superior to the SMA origin, the CT origin can be found.

Vascular preparation to isolate delivery to the kidneys: A total of three 4-0 nylon sutures (Ethicon) were used to temporarily ligate and isolate vessels at the following sites: two ties were used as a preventive measurement in case of bleeding, 1 cm distal to the left renal artery (i.e., for distal aorta ligation), one tie around the aorta between the SMA and CT (i.e., for proximal aorta ligation), and one tie around the SMA origin (i.e., for SMA ligation). Given that ligation of any vessel risks ischemic damage to the organ(s) which it supplies, our goal was to minimize both the number of vessels ligated and the time of any ligation. Although ligation of the aorta immediately superior and inferior to the renal arteries would be ideal, this was technically not possible due to the anatomical proximity of the SMA and CT to the origin of the right renal artery (FIG. 10B). However, with very careful dissection and mobilization of the posterior aorta, we were able to create enough room around the aorta between the CT and SMA to place a proximal suture. A distal suture was then placed around the aorta below the level of the left renal artery and another suture, around the SMA (FIG. 10B). None of the looped sutures were tied until immediately before the start of the injection; following sufficient repetition and practice we were able to limit any ischemia time (i.e., from suture ligation to un-ligation) to under 3 minutes. Once ready for injection, the looped sutures were then ligated in the following order: proximal aorta, SMA, and then distal aorta.

Injection technique: We determined that the optimal site of cannulation was immediately distal to the origin of the left renal artery, with the needle entering the vessel with a modest cranial angulation (FIG. 10B). Individual cannulation of the renal arteries was not possible due to their very small diameter. In our experience, we found that the largest bore needle which could be safely inserted into the aorta and then removed with hemorrhage control post-injection was a 34-gauge needle. Hence, a 34-gauge needle was then inserted into the aorta with gentle back-tension applied on the distal suture to help keep it in a perfect line to facilitate safe cannulation. Due to the proximity and downward-angle of the left renal artery, our initial studies showed that following injection of tattoo dye into the aorta, first the left kidney was completely stained with almost no staining of the right kidney. This was due to the longer distance from the needle tip to right renal artery, which was also more perpendicularly-angled compared to the left renal artery (FIG. 10A). Hence, we applied a metal clamp temporarily on the left renal artery for the first part of the injection to direct any injected solution preferentially into the right renal artery. After 50% of the solution is injected, the metal clamp was then removed and the reminder of the solution was injected which now preferentially flowed into the left kidney. At the end of the injection, the needle was removed and hemostasis was achieved following light pressure over the injection site for 1 minute with a Q-tip. The sutures were then un-ligated in the following order: distal aorta, SMA, and proximal aorta (i.e., distal to proximal) to prevent any barotrauma to the kidneys from the sudden inflow of blood. After confirming hemostasis, all intra-abdominal contents were returned to their original position and the abdominal wall closed using surgical staples. Animals were kept under warm light for 2 h after surgery until fully awake

### Measurements and Analysis of Kidney Function

Urine and blood samples were collected on day 12, at sacrifice. Blood was recovered by heart puncture in heparin-coated capillary blood collection tubes (Terumo), and it was centrifuged at 3,000 _{×} g for 10 min at 4° C. for measuring blood urea nitrogen (BUN), creatinine (Scr), NGAL, TNFα and IL-6 in plasma. The centrifugation step was repeated twice to minimize platelet contamination, and the clear plasma fraction was stored at -80° C. The levels of BUN concentrations were measured using the QuantiChrom Urea Assay Kit (DIUR-500, BioAssay Systems, Hayward, California, USA) and creatinine concentrations were measured using an enzyme-linked immunosorbent assay (ELISA; Stanbio, TX, USA). The levels of IL-6 and TNFα were measured by ELISA kit (R&D Systems) according to manufacturer’s instructions. Serum neutrophil gelatinase-associated lipocalin (NGAL) was measured using a NGAL Quantikine ELISA Kit (R&D Systems, USA). Urine samples were collected from both untreated control and both the treatment groups and evaluated for kidney injury molecule-1 (KIM-1), TIMP Metallopeptidase Inhibitor 1 (TIMP-1) and NGAL. The ELISA kit for KIM-1 and TIMP-1 were purchased from Cell signaling USA and samples were analyzed as per the manufacturer’s instructions and results were measured and calculated using ELISA reader at 450 nm (Bio-Rad, California, USA).

### Western Blotting

After sonication of the already sliced kidney tissues, cells were lysed using cell lysis buffer solution containing 50 mM Tris, pH 7.5, 0.3 M NaCl, 0.5% Triton X-100, 0.1% sodium azide with a mixture of protease inhibitor (Roche, USA) in 100 µL of buffer for 20 min at 4° C., then centrifuged at 18,500 _{×} g for 15 min. The pellet was discarded and the supernatant was kept for further analysis. Protein concentration was quantified using Micro-BCA (Thermo Scientific, USA) for both cell lysates and vesicle preparation in the presence of 0.2% SDS. Equivalent micrograms of proteins, in the case of tissues were homogenized in buffer 150 mM NaCl, 1 % Triton X-100, 0.5% deoxycholate, 0.1% SDS, 50 mM Tris and 1 mM EDTA. Proteins were separated on SDS-PAGE and transferred onto a nitrocellulose membrane. Membranes were then blocked with non-fat milk (5%) for 1 h and probed with primary antibodies at 4° C. overnight. Primary antibodies used in this study are as follows: AMPK (1:1000), phosphorylated AMPK (p-AMPK; 1:500), phosphorylated ERK (pERK; 1:1000), FGF2 (1:1000), Ki67 (1:1000), FGF23 (1:1000), NF-KkB (1:500) purchased from Santa Cruz Biotechnologies, USA. β-actin (Santa Cruz Biotechnology, Texas, USA) was used as an internal control. The membranes were then washed with PBS/0.01% Tween and incubated with anti-rabbit secondary antibody to IgG coupled to horseradish peroxidase at room temperature for 2 h (1:5000) (Santa Cruz Biotechnologies). Quantification was measured with the aid of chemiluminescence western blotting substrate (Roche) which enhance chemiluminescence, and ChemiDoc imager (Bio-Rad) was used to measure the intensity of the signals.

### Immunohistochemistry and Histology

The kidney tissues were fixed in 4% (vol/vol) paraformaldehyde in PBS for 24 h and later on imbedded in paraffin. Tissues were then processed and sectioned (6 µm) for immunostaining and histological staining. The sections were de-paraffinized and dehydrated by immersing them in graded ethanol concentrations for 1 h. Then antigens were retrieved using 10 mM sodium citrate buffer (pH 8.5) at 80° C. for 30 min. The tissue slices were blocked and permeabilized with PBS-T (0.2% Triton X-100 in PBS) containing 2% bovine serum for 30 min at room temperature. The sections were incubated overnight at 4° C. with the primary rabbit anti-human antibody to TNF- α, NF-kB, FGF2, Ki67 (1:200; Thermo Scientific, USA). Following incubation with Alexa 594-conjugated anti-rabbit antibody to immunoglobulin G (1:1000, Thermo Fisher Scientific, USA) and horseradish peroxidase (HRP) at room temperature for 2 h, DAB substrate (1 µg/mL, Cell Signaling, USA) was added to the sections let it stand for 30 min. Sections were stained using hematoxylin and eosin stain (Thermo Scientific) and observed under an optical microscope (Nikon, USA).

To determine the tubular casts score, hematoxylin & eosin-stained preparations were evaluated under a light microscope. Tubular casts were assessed in non-overlapping fields (up to 20 for each section) using 40 objective images, in a single-blind fashion. Scores were assigned by calculating the percentage of tubules positive for cast formation. Kidneys showing no injury were marked 0. Kidneys exhibiting minimal (<10%), mild (10-25%), moderate (26-50%), extensive (51-75%) and severe (≥75%) injuries were assigned scores of 1, 2, 3, 4 and 5, respectively.

### Real-Time PCR

The total RNA was extracted from kidney homogenized tissue using a Triazole Reagent (Sigma-Aldrich Missouri, USA). After digestion with DNase I, 2 µg RNA was reverse transcribed into cDNA as per the instructions in the Applied Biosystems reverse transcriptase kit (Applied Biosystems, CA, USA). The quality of cDNA was assessed by the ratio of the absorbance at 260 nm and 280 nm using an Agilent 2100 Bioanalyzer (Agilent Bioanalyzer, CA, USA). cDNA was then amplified by PCR in an iCycler Thermal Cycler (Bio-Rad, CA, USA) with SYBR Green (Applied Biosystems, CA, USA) and specific primers for KIM-1, TIMP-1, NAGL and NF-kB, GAPDH (Applied Biosystems, CA, USA) were used. The relative expression of mRNAs was calculated by the 2-^{ΔΔCt} method and normalized to glyceraldehyde 3-phosphate dehydrogenase (GAPDH).

### Statistical Analysis

The measured data was expressed as mean ± SD. Comparison between two or more groups is done by One-way ANOVA with Tukey’s multiple comparison test. Graph Pad Prism software (GraphPad, San Diego, CA), was used for all analysis, and the values of p<0.05 was considered statistically significant.

### Results

### IA Injection of Dye Selectively and Uniformly Labels the Kidneys

Our procedure for arterial exposure, vascular preparation, and injection are detailed in depth in the Methods section. Briefly, following exposure of the aorta, three sites were ligated with sutures: (1) the proximal aorta between the origins of the celiac trunk (CT) and superior mesenteric artery (SMA), (2) the SMA, and (3) the distal aorta (FIGS. 10B, 10C). A metal clamp is placed temporarily on the left renal artery to first allow delivery to the right renal artery; after injection of 50% of the volume, the clamp is removed, which results in preferential delivery to the left kidney due to natural flow.

Injection of tattoo dye into the distal aorta without described ligation technique resulted in non-targeted staining of the liver, lungs, and preferential staining of the left kidney due to the proximity and downward angle of the left renal artery origin (FIG. 10D). Injection of tattoo dye with our ligation and clipping technique resulted in targeted staining of both kidneys homogenously, without systemic staining (FIGS. 10E, 10F).

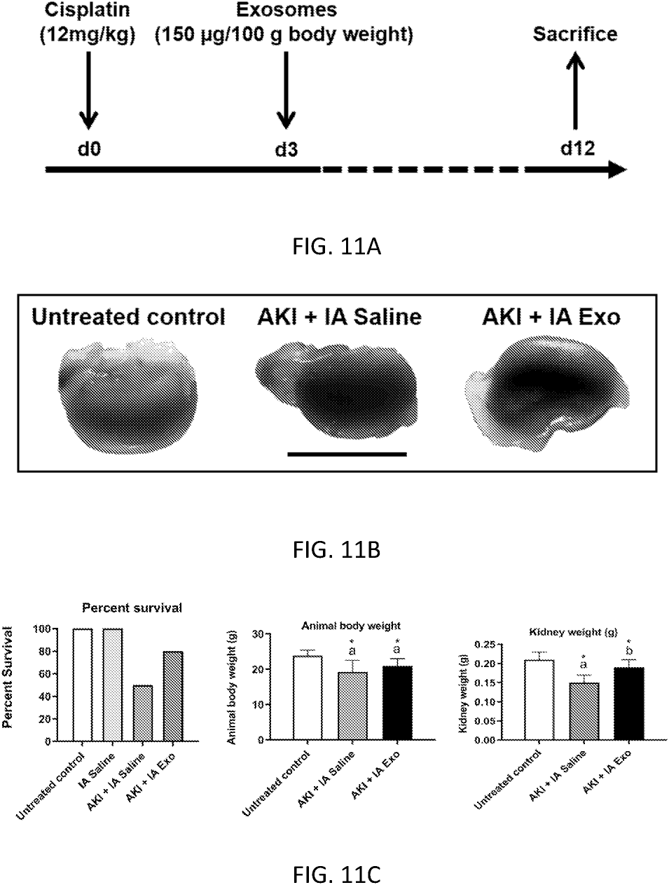

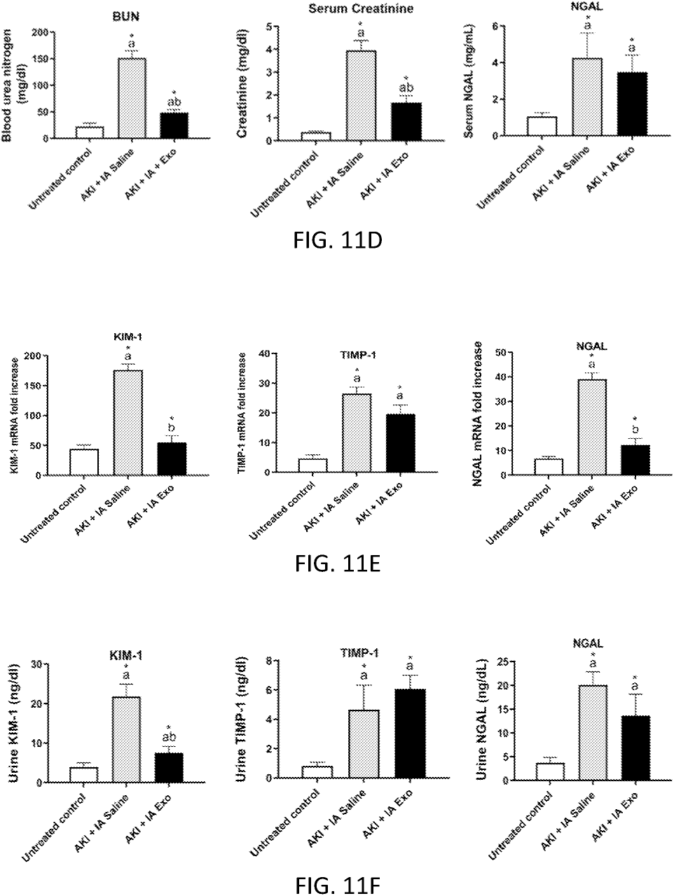

In our experience, the entire surgical procedure to deliver therapeutics, via IA injection into both kidneys, can be performed in approximately 35 minutes with no significant animal morbidity or mortality. Analysis of the serum from animals undergoing this procedure demonstrated no significant biochemical changes suggestive of kidney injury, including serum creatinine (SCr) and blood urea nitrogen (BUN) (Table 1). Any functionally significant iatrogenic kidney damage would have been detectable within this time period, without unnecessary sacrifice of animals for histologic biopsy. In addition, there was no mortality up to 12 days after the procedure in any of our experimental animals (FIG. 11C).

### IA Exosome Treatment Restores Kidney Function Following AKI

Following the induction of AKI with cisplatin and IA injection of saline, there was significant reduction in survival (100% vs. 50%), body weight (23.8±1.6 g vs. 19.2±3.4 g, p<0.05), and kidney weight (0.21±0.02 g vs. 0.15±0.02 g, p<0.05), with grossly visible atrophy and discoloration (FIGS. 11B, 11C) compared to untreated control animals. Compared to the AKI + IA saline group, animals treated with IA exosomes showed improved survival (50% vs. 80%), body weight (19.2±3.4 g vs. 20.9±2.1 g, p>0.05), and kidney weight (0.15±0.02 g vs. 0.19±0.02 g, p<0.05), as well as grossly-visible improvements in the size and color of the harvested kidneys.

Compared to untreated controls, animals in the AKI + IA saline group showed significantly elevated BUN (22.19±6.22 vs. 151.32±13.59 mg/dL, p<0.05), SCr (0.37±0.05 vs. 3.95±0.43 mg/dL, p<0.05) and serum NGAL (1.05±0.21 vs. 4.26±1.36 mg/mL, p<0.05) (FIG. 11D). Compared with the AKI + IA saline group, those treated with IA exosomes had significantly reduced BUN (151.32±13.59 vs. 48.28±5.87 mg/dL, p<0.05) and SCr (3.95±0.43 vs. 1.67±0.30 mg/dL, p<0.05) as well as decreased NGAL which did not reach statistical significance (4.26±1.36 vs.3.47±0.94 mg/mL; p>0.05).

Similar results were seen after measuring mRNA levels of injury molecules in the kidney tissue, KIM-1, TIMP-1, and NGAL. Compared to untreated controls, animals in the AKI + IA saline group showed significant upregulation in KIM-1 mRNA (176.2±9.8 vs. 44.2±6.4 relative expression, p<0.05), TIMP-1 (26.5±2.2 vs. 4.6±1.3 relative expression, p<0.05), and NGAL (37.0±2.5 vs. 6.7±0.9 relative expression, p<0.05). Compared to the AKI + IA saline animals, those treated with IA exosomes had significant downregulation of KIM-1 mRNA (176.2±9.8 vs. 55.0±11.3 relative expression, p<0.05) and NGAL mRNA (37.1±2.5 vs. 12.2±2.7 relative expression, p<0.05), as well as in TIMP-1 mRNA which did not reach statistical significance (26.5±2.2 vs. 19.6±3.0 relative expression, p>0.05) (FIG. 11D).

These trends were also recapitulated by measuring urine concentrations of kidney injury molecules. Compared to untreated controls, animals in the AKI + IA saline group had significant increases in urine KIM-1 (21.8±3.1 vs. 3.2±1.0 mg/dL, p<0.05), TIMP-1 (TIMP-1: 4.7±1.7 vs. 0.8±0.3 mg/dL, p<0.05), and NGAL (20.0±2.8 vs. 3.7±1.1 mg/dL, p<0.05) (FIG. 11E). Compared to the AKI + IA animals, treatment with IA exosomes significantly decreased urine KIM-1 (21.8±7.6 vs. 1.6±1.1 mg/dL, p<0.05), decreased urine NGAL though not reaching statistical significance (20.0±2.8 vs. 13.7±4.5 mg/dL, p>0.05), no effect on urine TIMP-1 (4.7±1.7 vs. 6.1±0.9 mg/dL, p>0.05).

### IA Exosome Treatment Attenuates Kidney Inflammation

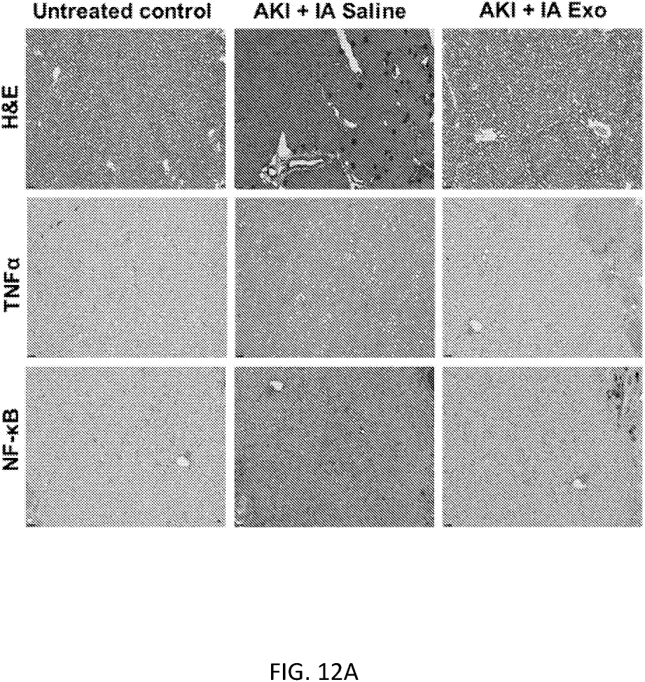

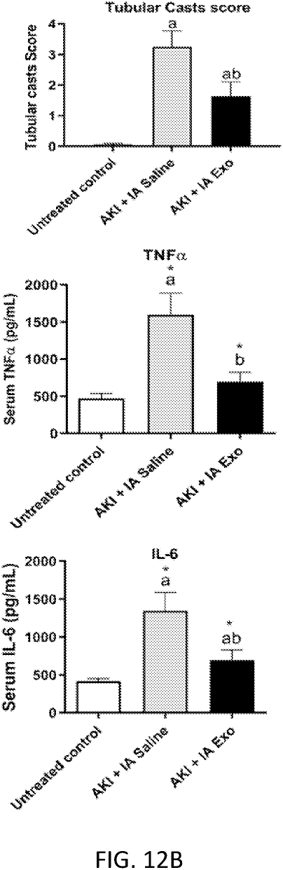

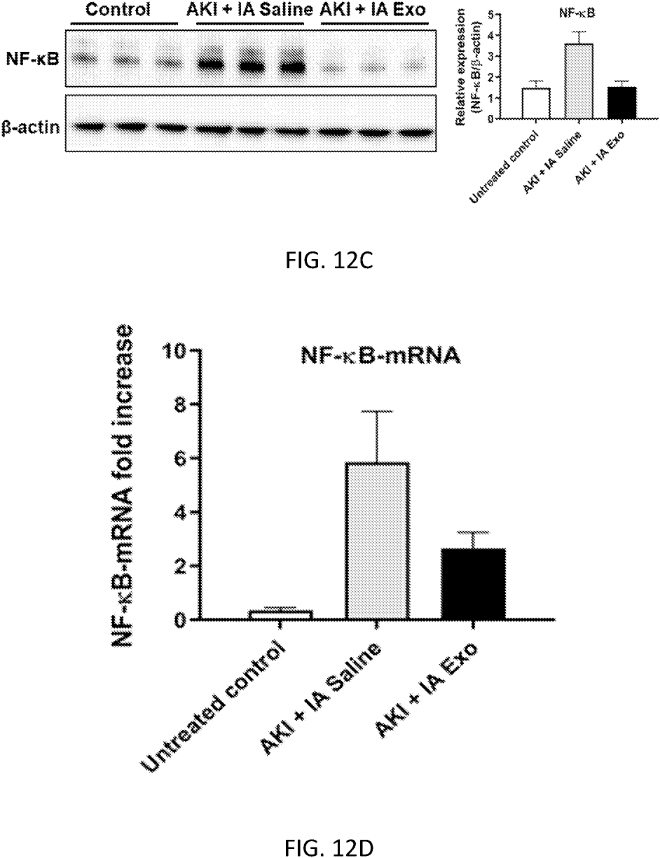

As inflammation is one of the major mechanisms by which AKI causes tissue damage, we analyzed the ability of IA exosomes to exert anti-inflammatory effects. Histological examination of kidney tissue from the AKI + IA saline group revealed extensive tubular cast formation compared to untreated control group (3.25±0.52 vs. 0.06±0.03 score, p<0.05), indicative of kidney injury (FIG. 12A). Mice treated with AKI + IA exosomes, however, showed significant decrease in casts formation compared to AKI + IA saline group (1.64±0.47 vs. 3.25±0.52 score, p<0.05). Immunohistochemical staining of the kidney showed that compared to untreated controls, those in the AKI + IA saline group had significant elevation of proinflammatory cytokine TNF-α and its downstream effector NF-kB (FIG. 12A). Treatment with IA exosomes appreciably decreased the signal of both these markers. Compared to untreated controls, serum levels of proinflammatory cytokines were significantly upregulated in the AKI + IA saline group, including TNF-α (469.2±68.8 vs. 1597.4±291.4 pg/mL, p<0.05) and IL-6 (414.9±35.1 vs. 1345.1±244.2 pg/mL, p<0.05) (FIG. 12B). Compared to the AKI + IA saline group, those treated with IA exosomes had significant downregulation of both TNF-α (698.7±125.3 vs. 1597.4±291.4 pg/mL, p<0.05) and IL-6 (694.5±244.2 vs. 1345.1±244.2 pg/mL, p<0.05).

Similar trends were seen in protein and mRNA expression of NF-kB, as measured in kidney lysate by Western blot and qRT-PCR, respectively (FIGS. 12C, 12D). Compared to the untreated controls, the AKI + IA saline group had upregulation of NF-kB protein (1.5±0.4 vs. 3.6±0.6 relative expression, p>0.05) and NF-kB mRNA (0.4±0.1 vs. 5.9±1.9 relative expression, p>0.05). Compared to the AKI + IA saline group, those treated with IA exosomes had downregulation of NF-kB protein (3.6±0.6 vs. 1.5±0.3 relative expression, p>0.05) and NF-kB mRNA (5.9±1.9 vs. 2.6±0.6 relative expression, p>0.05), though these differences did not reach statistical significance.

### IA Exosome Treatment Promotes Proliferation

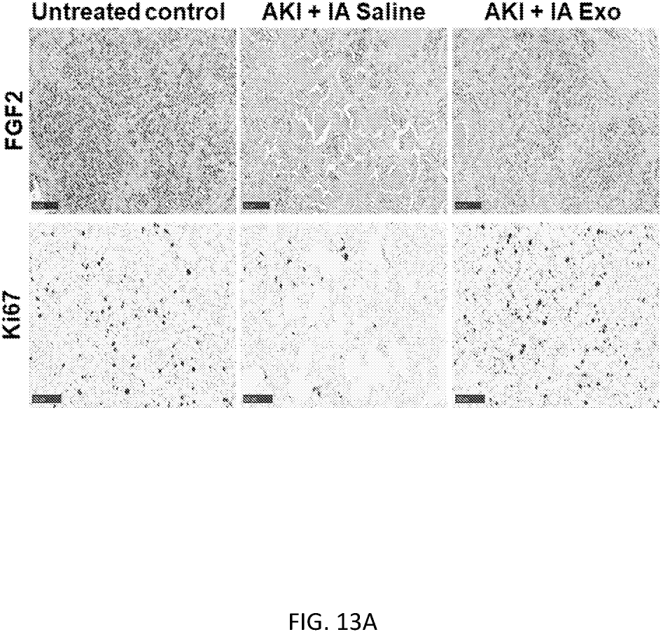

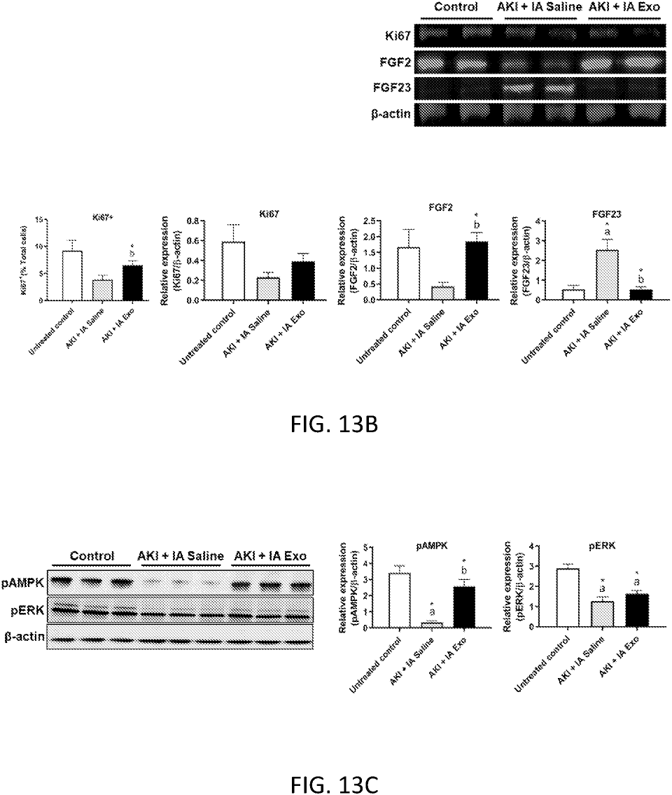

Cell proliferation is another major mechanism by which exosomes exert their therapeutic effect. Immunohistochemical staining of the kidney showed that compared to untreated controls, those in the AKI + IA saline group had lower expression of proliferation markers FGF2 and Ki67 (9.2±1.9% vs. 3.8±0.8% Ki67+, p>0.05) (FIG. 13A). Compared to the AKI + IA saline group, those treated with IA exosomes showed significant increase in Ki67+ cells (3.8±0.8% vs. 6.5±0.8% Ki67+, p<0.05).

These results were further corroborated via Western blot on kidney lysates. Compared to untreated controls, animals in the AKI + IA saline group had downregulation of Ki67 (0.6±0.2 vs. 0.2±0.1 relative expression, p>0.05) and FGF2 protein (1.7±0.6 vs. 0.4±0.1 relative expression, p>0.05), as well as upregulation of FGF23, a marker of kidney disease (0.5±0.2 vs. 2.5±0.5 relative expression, p<0.05) (FIG. 13B). Compared to AKI + IA saline group, those treated with IA exosomes showed upregulation of Ki67 (0.2±0.1 vs. 0.4±0.1 relative expression, p>0.05) and FGF2 (0.4±0.1 vs. 1.9±0.3 relative expression, p<0.05), as well as downregulation of FGF23 (2.1±0.5 vs. 0.5±0.1 relative expression, p<0.05).

To investigate the underlying signaling pathways responsible for these changes in proliferation, we conducted Western blot analysis of AMPK, a central regulator of cellular metabolism and growth, and ERK, a classical proliferation pathway. Compared to untreated controls, those in the AKI + IA saline group had significant downregulation of both pAMPK (3.4±0.4 vs. 0.3±0.1 relative expression, p<0.05) and pERK (2.9±0.2 vs. 1.3±0.2 relative expression, p<0.05) (FIG. 13C). Compared to the AKI + IA group, those treated with IA exosomes had significant upregulation of pAMPK (0.3±0.1 vs. 2.6±0.4 relative expression, p<0.05) and upregulation of pERK that did not reach statistical significance (1.3±0.2 vs. 1.6±0.2 relative expression, p>0.05).

### Discussion

In this study, we have developed a microsurgical technique for the injection of therapeutics into the mouse kidney, directly via its arterial blood supply. Our method is reproducible, while ensuring no operative morbidity or morality. Injection of tattoo dye using our method resulted in homogeneous staining of both kidneys, with almost no staining of off-target organs. We demonstrate the utility of our method by applying it to a mouse model of cisplatin-induced AKI. Intra-arterially delivered exosomes successfully restored physiological measures of kidney function, reduced histological and molecular markers of injury, attenuated local inflammation, and restored proliferative and regenerative signaling, demonstrating its potent regenerative potential.

Intra-arterial delivery of therapeutics to the kidney can be desirable for several reasons. These include minimizing systemic side effects while simultaneously increasing therapeutic efficacy; this can be attributed to increasing the delivery of therapeutics to the target site and avoiding of any potential first-pass metabolism for drugs or sequestration for cellular therapies. The development of IA therapeutics for kidney disease, however, relies on the availability of animal models and feasibility of surgical techniques. Previous studies that injected MSCs directly into the renal artery have used larger animal models, including cats [19], pigs [20], rats [21], and sheep [22]. Indeed, a meta-analysis of 21 studies applying MSC therapy to animal models of renal failure has suggested that IA delivery of MSCs has greater therapeutic effect compared to intravenous delivery [23]; it should be noted though that the studies used different animal and disease models, and none of them did a direct comparison between delivery routes. Regardless, these larger animals are often not readily accessible to researchers, or do not have as well-established models of kidney disease. Mice are by far the most comprehensive species in regards to models of renal pathology, with established models of acute kidney injury (AKI), chronic kidney disease (CKD), and kidney cancer, via nephrotoxic, genetic, autoimmune, metabolic, and ischemic etiologies [24, 25]. However, to our knowledge, there have not been any previous studies delivering therapeutics directly into the renal artery of mice [23], in part due to the technically challenging nature of injecting small vessels. Our technique thus paves the way for preclinical exploration of IA therapeutics for a wide range of renal pathologies.

As a demonstration of our technique, we applied it to a mouse model of cisplatin-induced AKI, treated with MSC-derived exosomes. Exosomes carry a cargo of therapeutic molecules that suppress inflammation and apoptosis and promote regeneration, and have shown promising preclinical results for treating AKI [15]. Since exosomes are not known to home to sites of injury like MSCs [26, 27], locoregional delivery may be one of the most direct ways to optimize exosome therapy. Our IA delivery of exosomes restored physiological markers of kidney function (BUN, creatinine), reduced histological and molecular markers of kidney injury (KIM-1, TIMP-1, NGAL, FGF23) [28], attenuated inflammation (TNF-α, IL-6), and spurred cellular proliferation (Ki67, FGF2). These effects may have been mediated through AMPK, which has a documented role in kidney regeneration by modulating energy metabolism, inflammation, and stress [29], as well as MAPK/ERK, a classical pathway promoting cellular proliferation [30]. Taken together, these results offer proof of principle of the feasibility and efficacy of IA delivery of therapeutics into the kidney. Of immediate interest are future studies comparing the effect of different MSC-based therapies (whole MSCs vs. MSC-derived exosomes) through different administration routes (intravenous, intra-arterial, or intraperitoneal). Such knowledge is critical for the translation of preclinical studies into feasible and effective therapeutics.

Finally, a point on the clinical-translational aspect of our technique deserves brief discussion here. Obviously, our microsurgical technique is quite invasive to the mouse, requiring opening of the peritoneal cavity and exposure of the abdominal aorta. However, establishing access to the renal artery in humans is relatively simple given modern interventional radiology techniques. Percutaneous access to the renal artery is easily achieved through the femoral, brachial, or radial arteries, and such techniques have long been in use for the treatment of conditions like renal artery stenosis [31]. Exosomes may outperform cellular therapies in this application, as their smaller size reduces the risk of embolic events and can be injected through catheters with less damage and shear stress. Thus, our technique is one that allows rapid translation to clinical trials given the correction indications.

In summary, we have developed the first known microsurgical technique for the direct delivery of therapeutics into the mouse kidney via its arterial blood supply. This technique should now allow researchers to study the effects of direct delivery of different oncological and regenerative therapies into the kidneys within the many well-established mouse models of renal pathology, with rapid clinical translation possible through modern interventional radiology techniques.

TABLE 1

Comparative markers of renal function pre- and post-injection. Serum creatinine (SCr) and blood urea nitrogen (BUN) in mice at baseline and 24 hr after intra-arterial injection of saline.

| | Reference ranges | Baseline | Post-operative (24 hr) |
|---|---|---|---|
| SCr (mg/dL) | 0.1-1.1 | 0.2±0.1 | 0.1±0.1 |
| BUN (mg/dL) | 20.3-24.7 | 26.0±2.3 | 28.0±6.4 |

### References

1. NIDDK. Kidney Disease Statistics for the United States. 2016; Available from: niddk.nih.gov/health-information/health-statistics/kidney-disease.

2. Siegel, R.L., K.D. Miller, and A. Jemal, Cancer statistics, 2020. CA Cancer J Clin, 2020. 70(1): p. 7-30.

3. Papazova, D.A., et al., Cell-based therapies for experimental chronic kidney disease: a systematic review and meta-analysis. Dis Model Mech, 2015. 8(3): p. 281-93.

4. Morigi, M., C. Rota, and G. Remuzzi, Mesenchymal Stem Cells in Kidney Repair. Methods Mol Biol, 2016. 1416: p. 89-107.

5. Rangel-Castilla, L., et al., Acute stroke endovascular treatment: tips and tricks. J Cardiovasc Surg (Torino), 2016. 57(6): p. 758-768.

6. Dind, A., et al., Contemporary Management of ST-Elevation Myocardial Infarction. Heart Lung Circ, 2017. 26(2): p. 114-121.

7. Habib, A., et al., Locoregional therapy of hepatocellular carcinoma. Clin Liver Dis, 2015. 19(2): p. 401-20.

8. Morigi, M. and P. De Coppi, Cell therapy for kidney injury: different options and mechanisms--mesenchymal and amniotic fluid stem cells. Nephron Exp Nephrol, 2014. 126(2): p. 59.

9. Lai, R.C., R.W. Yeo, and S.K. Lim, Mesenchymal stem cell exosomes. Semin Cell Dev Biol, 2015. 40: p. 82-8.

10. Schrepfer, S., et al., Stem cell transplantation: the lung barrier. Transplant Proc, 2007. 39(2): p. 573-6.

11. Santeramo, I., et al., Human kidney-derived cells ameliorate acute kidney injury without engrafting into renal tissue. Stem Cells Transl Med, 2017. 6(5): p. 1373-1384.

12. Leibacher, J. and R. Henschler, Biodistribution, migration and homing of systemically applied mesenchymal stem/stromal cells. Stem Cell Res Ther, 2016. 7: p. 7.

13. Eggenhofer, E., et al., Mesenchymal stem cells are short-lived and do not migrate beyond the lungs after intravenous infusion. Front Immunol, 2012. 3: p. 297.

14. Fischer, U.M., et al., Pulmonary passage is a major obstacle for intravenous stem cell delivery: the pulmonary first-pass effect. Stem Cells Dev, 2009. 18(5): p. 683-92.

15. Lv, L.L., et al., Therapeutic application of extracellular vesicles in kidney disease: promises and challenges. J Cell Mol Med, 2018. 22(2): p. 728-737.

16. Phinney, D.G. and M.F. Pittenger, Concise Review: MSC-Derived Exosomes for Cell-Free Therapy. Stem Cells, 2017. 35(4): p. 851-858.

17. Ullah, M., D.D. Liu, and A.S. Thakor, Mesenchymal Stromal Cell Homing: Mechanisms and Strategies for Improvement. iScience, 2019. 15: p. 421-438.

18. Kim, D.K., et al., Chromatographically isolated CD63+CD81 + extracellular vesicles from mesenchymal stromal cells rescue cognitive impairments after TBI. Proc Natl Acad Sci U S A, 2016. 113(1): p. 170-5.

19. Thomson, A.L., et al., Intra-arterial renal infusion of autologous mesenchymal stem cells for treatment of chronic kidney disease in cats: Phase I clinical trial. J Vet Intern Med, 2019. 33(3): p. 1353-1361.

20. Zhang, L., et al., Selective intrarenal delivery of mesenchymal stem cell-derived extracellular vesicles attenuates myocardial injury in experimental metabolic renovascular disease. Basic Res Cardiol, 2020. 115(2): p. 16.

21. De Martino, M., et al., Mesenchymal stem cells infusion prevents acute cellular rejection in rat kidney transplantation. Transplant Proc, 2010. 42(4): p. 1331-5.

22. Behr, L., et al., Intra renal arterial injection of autologous mesenchymal stem cells in an ovine model in the postischemic kidney. Nephron Physiol, 2007. 107(3): p. p65-76.

23. Wang, Y., et al., Systematic review and meta-analysis of mesenchymal stem/stromal cells therapy for impaired renal function in small animal models. Nephrology (Carlton), 2013. 18(3): p. 201-8.

24. Hewitson, T.D., T. Ono, and G.J. Becker, Small animal models of kidney disease: a review. Methods Mol Biol, 2009. 466: p. 41-57.

25. Bao, Y.W., et al., Kidney disease models: tools to identify mechanisms and potential therapeutic targets. Zool Res, 2018. 39(2): p. 72-86.

26. Herrera, M.B., et al., Mesenchymal stem cells contribute to the renal repair of acute tubular epithelial injury. Int J Mol Med, 2004. 14(6): p. 1035-41.

27. Morigi, M., et al., Mesenchymal stem cells are renotropic, helping to repair the kidney and improve function in acute renal failure. J Am Soc Nephrol, 2004. 15(7): p. 1794-804.

28. Wahl, P. and M. Wolf, FGF23 in chronic kidney disease. Adv Exp Med Biol, 2012. 728: p. 107-25.

29. Rajani, R., N.M. Pastor-Soler, and K.R. Hallows, Role of AMP-activated protein kinase in kidney tubular transport, metabolism, and disease. Curr Opin Nephrol Hypertens, 2017. 26(5): p. 375-383.

30. Zhang, W. and H.T. Liu, MAPK signal pathways in the regulation of cell proliferation in mammalian cells. Cell Res, 2002. 12(1): p. 9-18.

31. White, C.J., Catheter-based therapy for atherosclerotic renal artery stenosis. Prog Cardiovasc Dis, 2007. 50(2): p. 136-50.

### Example 3 Pulsed Focused Ultrasound Enhances the Therapeutic Effect of Mesenchymal Stromal Cell-Derived Extracellular Vesicles in Acute Kidney Injury

### Introduction

Acute kidney injury (AKI) is a condition characterized by a rapid deterioration of kidney function and is a common problem in hospitalized patients as well as those with co-morbid chronic diseases [1]. In the past few decades, there has been a substantial increase in hospitalizations for AKI, with the Centers for Disease Control and Prevention (CDC) estimating the number hospitalizations in the USA increasing over fourfold from 953,926 in 2000 to 3,959, 560 in 2014 [2]. Studies in other countries have shown similar trends, with dialysis-treated AKI reportedly increasing more than thirteen-fold in England from 1998 to 2013 [3] and approximately threefold in Denmark from 2000 to 2012 [4]. AKI is especially prevalent among critically ill patients and is often secondary to another pathology. In addition to being an independent risk factor for end-stage renal disease (ESRD) and death [5], multiple studies have shown that AKI may initiate the development of chronic kidney disease (CKD) or, where CKD is already present, accelerate its worsening [5-10]. Treatment for AKI is predominantly supportive, aimed at maintaining volume homeostasis and correcting biochemical abnormalities. Hence, there is an urgent need for new therapies that might actually repair injured kidneys and prevent the progression to worsening kidney disease.

Recently, there has been interest in the regenerative properties of mesenchymal stromal cell (MSC) therapy for the repair and regeneration of damaged kidney tissue [11, 12]. MSCs are a heterogeneous population of cells found in various adult tissues. Due to their immuno-modulatory and regenerative properties, as well as their ease of isolation and in vitro expansion, MSCs have been extensively explored as a platform for cellular therapy [13]. Early animal studies have shown that MSCs are capable of homing to damaged kidney tissue and contributing to renal repair [14, 15]. Recently, it has become clear that the therapeutic effects of MSCs come not from their ability to directly differentiate into new cells, but rather by the release of soluble factors and extracellular vesicles (EVs) with regenerative properties [16]. These molecules act through paracrine signaling to stimulate repair via anti-inflammatory, mitogenic, vasculotropic, and pro-survival pathways, which provide protection for surviving intrinsic epithelial cells and promote their proliferation [17, 18]. Since MSCs act via paracrine signaling, their proximity to the injured site is believed to be crucial for tissue regeneration. However, multiple studies have shown that when MSCs are injected intravenously, they are predominantly trapped in the lung microvasculature, in what is termed the pulmonary first-pass effect [19-23]. Consequently, instead of using whole cells, many studies have now begun investigating the use of purified EVs from MSC as a cell-free therapy [24], which are small enough to avoid pulmonary trapping [25]. MSC-derived EVs carry a cargo of regenerative molecules and have been shown to have a therapeutic effect in various animal models of disease [26, 27].

Despite the growing interest in EVs for regenerative applications, there remains an unmet need to improve their therapeutic effect. One promising avenue of optimization could be the use of pulsed focused ultrasound (pFUS) [28]. pFUS uses short-duration, high-intensity pulses of sound waves to non-destructively sonicate target tissues. When combined with imaging guidance, pFUS can be targeted precisely to locations deep within the body. Previous studies have shown that pFUS can enhance MSC therapy for AKI [29, 30], but different mechanisms have been proposed for this effect. The homing hypothesis posits that sound waves transiently upregulate local inflammatory and chemoattractive signals [31-33]. This local gradient of cytokines, chemokines, and trophic factors has been shown to promote the homing of MSCs to various tissues including the skeletal muscle [34, 35] and the kidney [29, 30, 32, 36]. In the setting of a mouse model of cisplatin-induced AKI, this increased homing of MSCs after pFUS was able to improve renal function more so than MSCs alone [30]. However, we have previously demonstrated that pFUS can enhance MSC therapy without local upregulation of cytokines or increased MSC homing [29]. Instead, pFUS was shown to act through protein intermediates to spur endogenous proliferation pathways, an effect which synergized with MSCs. These two mechanisms may not necessarily be at odds with one another, but may instead reflect differences in ultrasound parameters leading to differences in subsequent bioeffects [28]. Although there have been many studies on the effect of pFUS on MSC therapy, little is known about its effect on EV therapy. Hence, in this study, we use a mouse model of cisplatin-induced AKI to assess the effect of pFUS on MSC-derived EV therapy.

### Methods

### Extracellular Vesicle Isolation, Characterization, and Purification

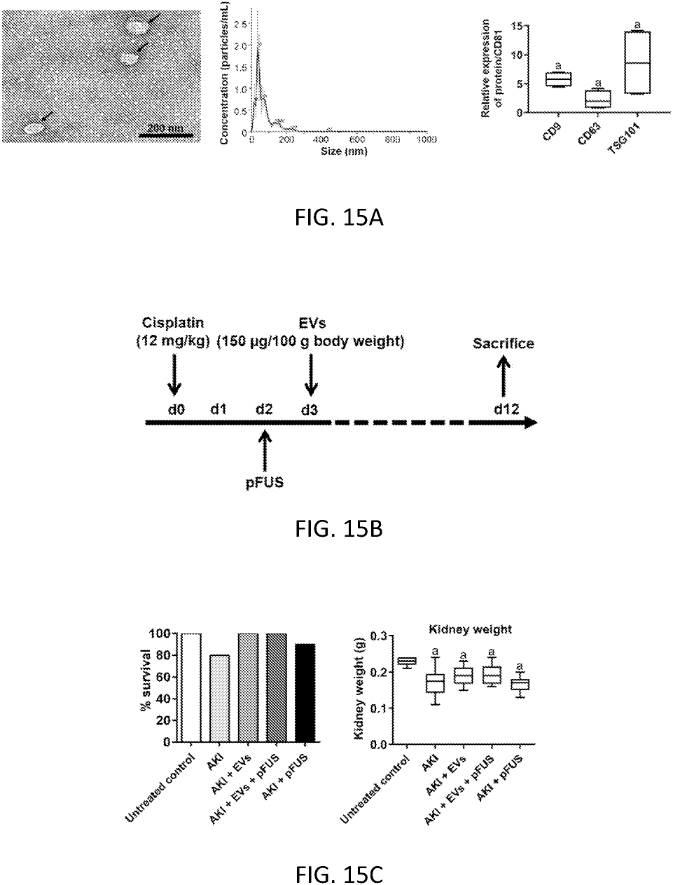

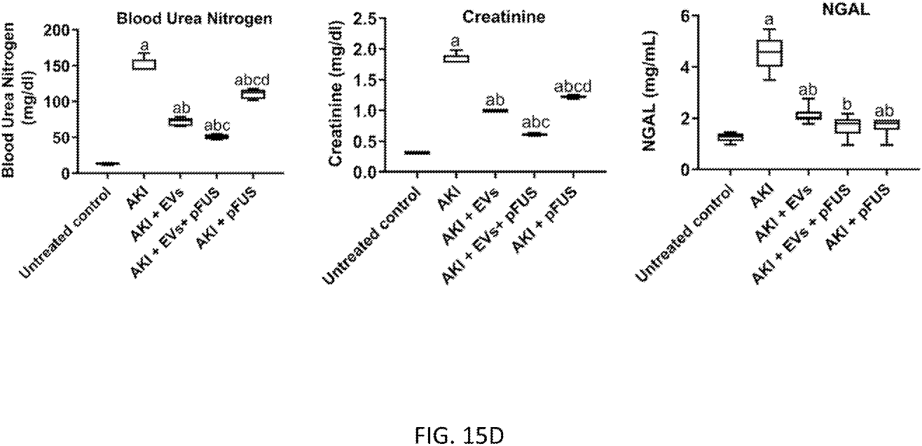

Human bone marrow-derived MSCs (BM-MSCs) pooled from three donors were purchased from ATCC (PCS-500-012), transduced with GFP, and cultured in tissue culture flasks containing proliferative medium containing 10% fetal bovine serum (FBS), Dulbecco’s modified Eagle medium (DMEM), 100 µg/mL penicillin/streptomycin, and 2 mmol/mL glutamine (Thermo Fisher Scientific, USA) at 5% CO₂ until passage 4, with culture media change every 2 days. The BM-MSCs were then plated in a flask at 2 _{×} 10⁴ cells/cm² with media containing DMEM, 100 µg/mL penicillin/streptomycin, 2 mmol/ mL glutamine, and 3% EV-free FBS for the isolation of EVs. After overnight incubation, the conditioned media were centrifuged at 4° C. at 300xg for 10 min to remove dead cells, 17,000xg for 10 min to remove cellular debris, and 110,000xg for 90 min to pellet EVs. EVs were washed with PBS and ultracentrifuged again before use. We then applied this medium at room temperature to a column containing the anion exchange resin (Q Sepharose Fast Flow, GE Healthcare, IL, USA) balanced with 50 mM NaCl in 50 mM phosphate buffer and washed with 100 mM NaCl in 50 mM phosphate buffer and then rinsed with 500 mM NaCl in 50 mM phosphate buffer. EV fractions (20-30 mL) were pooled, dialyzed against PBS, filter sterilized, and used for this study. Nanoparticle tracking analysis (NTA) was performed using Nano-Sight NS300 (ATA Scientific, Australia) for assessment of the size and number of the isolated EVs. NTA 3.3 Software was used for measurements and data output. The mean diameter was 118 nm and standard deviation 27 nm, with sizes ranging from 20 to 180 nm (FIG. 15A). EVs were characterized by expression of the following human protein markers: CD9, CD63, CD81, and TSG101, all of which were positive.

### Animal Experiments

All experimental procedures were performed in accordance with the guidelines and regulations of the Administrative Panel on Laboratory Animal Care (APLAC) at Stanford University. A total of 50 female CD1 mice (8 weeks old; body weight 30 ± 2 g) were purchased from Charles River Laboratories (Wilmington, MA, USA). All mice were housed for 1 week before any experiment in controlled conditions with 12-h light-dark cycles. EVs were isolated and purified by chromatography as previously described [37]. To investigate the protective effect of EVs and pFUS in cisplatin-induced AKI, animals were injected with cisplatin on day 0, administered pFUS on day 2, and treated with EVs on day 3 (FIG. 15B).

CD1 mice were randomly divided into 5 experimental groups, with each group containing 10 animals. Group 1 consisted of untreated control animals. For groups 2-5, animals received a single injection of cisplatin (12 mg/kg intraperitoneally) on day 0 to induce AKI. Group 2, AKI control, received no treatment; group 3, EVs alone, received EV treatment at a dose of 150 µg/100 g body weight via a tail vein injection on day 3; group 4, pFUS + EVs, received pFUS on day 2 followed by EVs on day 3; and group 5, pFUS alone, received pFUS only on day 2. At day 12 post-cisplatin injection, mice were sacrificed using an intraperitoneal injection of ketamine (100 mg/ kg) and xylazine (10 mg/kg) followed by cervical dislocation, at which point blood and kidney samples were collected. For blood samples, the blood was collected to obtain serum which was then stored at - 20° C. for further analysis. One kidney was immediately immersed in 10% neutral buffered formalin for histological analysis while the other kidney was immediately frozen in liquid nitrogen for biochemical marker measurement and Western blot analysis. Subsequent analyses were conducted in a single-blind manner wherever possible.

### Pulsed Focused Ultrasound

A 1.1-MHz central frequency custom high-intensity focused ultrasound (HIFU) therapy transducer (H-102NRE, Sonic Concepts, Bothell, WA, USA) with 49-mm central opening was used. The HIFU transducer was calibrated in a water tank filled with degassed and deionized water. The transducer was driven by an Agilent 33250A function generator (Agilent Technologies, Santa Clara, CA, USA) and connected to a 50-dB ENI 525LA linear power amplifier (ENI Technology, Inc., Rochester, NY, USA) and an impedance matching circuit (Sonic Concepts, Bothell, WA, USA). The transducer was excited at central 1.1-MHz frequency with 20 cycles at 100-Hz pulse repetition frequency (PRF) in a “burst” mode. A hydrophone (HNR-0500, Onda Corporation, Sunnyvale, CA, USA) was placed in the focal spot of the transducer (55 mm away from its surface) and an Acoustic Intensity Measurement System (AIMS III, Onda Corporation, Sunnyvale, CA, USA) was used for precise movement and positioning of the hydrophone and digitizing the waveforms. The measured beam profile (full width half-maximum area for pressure) at the focal area was 10 mm long and 1.5 mm in diameter. The intensity and pressure measurements were performed for negative peak pressures (NPP) up to 3 MPa in order to reduce risks related to hydrophone damage. The obtained intensities and NPP values were then scaled to the desired PRF and duty cycle (DC) and linearly extrapolated to higher pressures/intensities.

For image-guided therapy to the mouse kidneys, a setup of co-aligned transducers was employed. The imaging transducer (Siemens Acuson S2000 14 L5 sp., Siemens Corporation, WA, USA) was placed in the central opening of the HIFU transducer. Both transducers were then aligned and fixed in a custom-made 3D-printed holder. The HIFU transducer’s focal spot was fixed at 55 mm axial, 0 mm lateral distance from the central point of the imaging transducer. Any misalignment of the HIFU and imaging beam was checked several times in the water tank with the hydrophone and oscilloscope by assembling and disassembling the 3D-printed holder. On all measurements, the measured beam misalignment was less than 200 µm. The ultrasound guidance of the in vivo kidney therapy was done with the Siemens S2000 scanner (Siemens Medical Solutions, Issaquah, WA, USA). Mice were kept under isoflurane anesthesia (2.5% induction, 0.1% maintenance) and submerged vertically in the water tank with the head kept above the water surface and body temperature maintained at 37° C. The assembled holder with the HIFU and imaging transducers was connected to a translation stage and kept in the water at approximately 50 mm axial distance from the mouse. The mouse’s kidney was identified on the Siemens S2000 scanner and placed at the desired location, 55 mm axially and 0 mm laterally from the central point of the imaging transducer. To treat the whole kidney, 8 non-overlapping adjacent regions through the kidney were targeted for 30 s per region. The time to treat one kidney with these parameters was approximately 4 min. In order to deliver pFUS therapy to the animal, the HIFU transducer was used with the following parameters: 5% DC, 5 Hz PRF, 2.9 MPa PNP, and 272 W/cm² spatial average pulse average intensity (_{ISAPA}). After pFUS treatment, each mouse was removed from the water bath, dried, and placed in a recovery cage.

### Analysis of Kidney Function

Blood samples were collected retro-orbitally from anesthetized mice for analysis of biochemical levels, including blood urea nitrogen (BUN) and creatinine (Cr) using reagents purchased from Santa Cruz biotechnology USA according to the manufacturer’s instructions mentioned in the ELISA kit. Serum neutrophil gelatinase-associated lipocalin (NGAL) was measured using a mouse NGAL Quantikine ELISA Kit (R&D Systems, USA) according to the manufacturer’s instructions. The ELISA kit for kidney injury molecule-1 (KIM-1) and TIMP metallopeptidase inhibitor 1 (TIMP-1) were purchased from Cell signaling USA, samples were analyzed according to the manufacturer’s instructions, and results were measured and calculated at 450 nm via an ELISA reader (Bio-Rad, CA, USA) within 10 min. Urine samples were collected from both the control and AKI groups and also evaluated for KIM-1, TIMP-1, NGAL, and creatinine. Renal tissues were also homogenized, and the supernatant removed after 15 min of centrifugation at 3000xg at 4° C. Next, NGAL and KIM-1 levels were measured using commercial kits according to the manufacturer’s instructions (R&D Systems, USA).

### Histology, Immunohistochemistry, and Immunofluorescence

At day 12, the mice were sacrificed and then perfused with 4% (vol/vol) paraformaldehyde in PBS to remove the blood from the tissues. Next, whole kidney tissues were fixed for 24 h in 4% (vol/vol) paraformaldehyde After fixation and paraffin embedding, the kidney tissues were then sectioned into 6-µm slices for hematoxylin-eosin and trichrome staining. The slides were also stained with 4',6-diamidino-2-phenylindole (DAPI) (D1306, Thermo Fisher Scientific, Santa Clara, CA, USA) and processed for GFP signal quantification using ImageJ software (NIH, USA) and flow cytometry as described previously [38].

### Pathological Score for Tubular Injury

To determine kidney injury and rescue at the tissue level, we used a semi-quantitative histological scoring method previously defined by Klopfleisch et al. [39]. Injury was defined by dilated tubules, glomerular casts, tubular casts, necrosis, and tubular degeneration. The number of casts and tubular profiles showing necrosis were recorded in a single-blind fashion. Score 0 represents injury area less than 10%, whereas scores 1, 2, 3, and 4 represent injury involving 10-25%, 25-50%, 50- 75%, or > 75% of the visualized field, respectively. At least 20 random fields at _{×} 200 magnification were evaluated for each mouse with the average score calculated. For immunohistochemical staining, paraffin-embedded kidney sections were deparaffinized, hydrated, and antigen-retrieved, and endogenous peroxidase activity was quenched by 3% _{H202.} Sections were then blocked with 10% normal donkey serum, followed by incubation with different antibodies such as interleukin 6 (IL-6) (M620, Thermo Fisher Scientific, Santa Clara, CA, USA) and tumor necrosis factor alpha (TNF-α) (M3TNFAI, Thermo Fisher Scientific, Santa Clara, CA, USA) overnight at 4° C. After incubation with a secondary antibody for 1 h, sections were incubated with 3-3'-diaminobenzidine (DAB) (H-2200-30, Vector Laboratories, CA, USA). Slides were viewed with a microscope equipped with a digital camera (Nikon Eclipse 80i, USA) and then analyzed with a NanoZoomer (NanoZoomer S360Hamamatsu, USA).

### Western Blot Analysis

Kidney tissue was sliced into thin sections, followed by sonication and homogenization. The lysate was then placed in 1 _{×} SDS sample buffer in association with radioimmunoprecipitation assay (RIPA) buffer solution containing 1% NP40, 0.1% SDS, 100 mg/ml PMSF, 1% protease inhibitor cocktail, and 1% phosphatase I and II inhibitor cocktail (Sigma, St Louis, MO, USA) on ice. The supernatant was then collected following centrifugation at 13,000xg at 4° C. for 30 min. Protein concentration was determined by a bicinchoninic acid protein assay. An equal amount of protein was loaded into 10% or 15% SDS-PAGE and transferred onto polyvinylidene difluoride membranes. The primary antibodies were used at 1:200 dilution unless otherwise noted: Akt (2920S, Cell Signaling Technology, MA, USA), p-Akt (PA5-95669, Thermo Fisher Scientific, Santa Clara, CA, USA), KIM-1 (PA5-98302, Thermo Fisher Scientific, Santa Clara, CA, USA), NGAL (ABS 043-29-02, Thermo Fisher Scientific, Santa Clara, CA, USA), TIMP-1 (MA5-13688, Thermo Fisher Scientific, Santa Clara, CA, USA), TNF-α (MM350C, Thermo Fisher Scientific, Santa Clara, CA, USA), NF-kB (PA5-16545, Thermo Fisher Scientific, Santa Clara, CA, USA), endothelial nitric oxide synthase (eNOS) (5880S, Cell Signaling Technology, MA, USA), SIRT3 (8469S, Cell Signaling Technology, MA, USA), PCNA (13-3900, Thermo Fisher Scientific, Santa Clara, CA, USA), VEGF (MA5-13182, Thermo Fisher Scientific, Santa Clara, CA, USA), survivin (PA1 -16836, Thermo Fisher Scientific, Santa Clara, CA, USA), Bcl-2 (MA5-11757, Thermo Fisher Scientific, Santa Clara, CA, USA), BAX (MA5-14003, Thermo Fisher Scientific, Santa Clara, CA, USA), Caspase-3 (MA5-11521, Thermo Fisher Scientific, Santa Clara, CA, USA), p-ERK (MA5-15705, Thermo Fisher Scientific, Santa Clara, CA, USA), ERK (13-8600, Thermo Fisher Scientific, Santa Clara, CA, USA), p-MEK (sc-271914, Santa Cruz Biotechnology, TX, USA), MEK (sc6250, Santa Cruz Biotechnology, TX, USA), and anti-β-actin (sc-1616, 1:1000 dilution, Santa Cruz Biotechnology, TX, USA). Quantification was performed by measuring the intensity of the signals with the aid of the National Institutes of Health Image software package and Bio-Rad image software (Bio-Rad USA).

### Real-Time PCR

RNA was extracted from kidney homogenized tissue using a Triazole Reagent (Sigma-Aldrich MO, USA). After digestion with DNase I, 2 g RNA was reverse transcribed with the Applied Biosystems reverse transcriptase kit (Applied Biosystems, CA, USA). The quality of cDNA was assessed by the ratio of the absorbance at 260 nm and 280 nm using an Agilent 2100 Bioanalyzer (Agilent Bioanalyzer, CA, USA). cDNA was then amplified by PCR in an iCycler Thermal Cycler (Bio-Rad, CA, USA) with SYBR Green (Applied Biosystems, CA, USA) and specific primers for Kim1 (Mm01291075_m1, Thermo Fisher Scientific, Santa Clara, CA, USA), Timp1 (Mm01341361_m1, Thermo Fisher Scientific, Santa Clara, CA, USA), Ngal(Mm00443258_m1, Thermo Fisher Scientific, Santa Clara, CA, USA), IL6 (Mm00446190_m1, Thermo Fisher Scientific, Santa Clara, CA, USA), Tnfa (Mm00443258_m1, Thermo Fisher Scientific, Santa Clara, CA, USA), Nfkb1 (Mm00476361_m1, Thermo Fisher Scientific, Santa Clara, CA, USA), Gapdh (Mm99999915_g1, Thermo Fisher Scientific, Santa Clara, CA, USA), and 18S (Mm02601777_ g1, Thermo Fisher Scientific, Santa Clara, CA, USA); the latter two were used as housekeeping genes. The expression of marker genes was normalized to the endogenous GAPDH expression level and calculated with the 2^{-ΔΔCt} formula in % GAPDH expression [40].

### Statistical Analysis

All data are expressed as the mean ± standard deviation (SD) and analyzed with analysis of variance (ANOVA) followed by the Tukey test. Statistical analysis was performed using the GraphPad Prism 8.0 software (Graph-pad Software, Inc., San Diego, USA).

### Results

### EVs and pFUS Reduce the Clinical Manifestations of AKI

EVs were confirmed to be positive for the expression of CD9, CD63, and TSG101, with quantification normalized to the internal control CD81 (CD9: 5.77 ± 1.34 relative expression; CD63: 2.25 ± 1.66 relative expression; TSG101: 8.64 ± 6.10 relative expression) (FIG. 15A). Cisplatin was administered to induce AKI on day 0, after which animals were treated with pFUS on day 2 or EVs on day 3 (FIG. 15B). There was a reduction in the survival of animals in the AKI (80%) and pFUS alone (90%) groups at day 12, compared to untreated controls (100%) (FIG. 15C). However, animals treated with EVs alone or pFUS + EVs showed no mortality at day 12. A similar trend was observed in kidney weight following cisplatin administration: compared to untreated controls, kidney weight was significantly reduced among animals in the AKI (0.17 ± 0.04 vs. 0.23 ± 0.01 g, p < 0.05) and pFUS alone groups (0.17 ± 0.02 vs. 0.23 ± 0.01 g, p < 0.05). Compared to the AKI group, treatment with EVs alone partially ameliorated this reduction in kidney weight (0.19 ± 0.03 vs. 0.17 ± 0.04 g, p > 0.05) (FIG. 15C). These trends were also reflected in serum laboratory values. At day 12, animals in the AKI group showed significantly elevated levels of several kidney injury markers compared to untreated controls, including blood urea nitrogen (BUN) (13.27 ± 1.26 vs. 150.23 ± 9.62 mg/dL, p < 0.05), creatinine (SCr) (0.31 ± 0.01 vs. 1.83 ± 0.08 mg/ dL, p < 0.05), and NGAL (1.25 ± 0.19 vs. 4.54 ± 0.68 mg/ mL, p < 0.05) (FIG. 15D). Compared to AKI, treatment with EVs alone significantly reduced the levels of all three injury markers (BUN 150.23 ± 9.26 vs. 72.23 ± 5.61 mg/dL, p < 0.05; SCr 1.83 ± 0.08 vs. 0.99 ± 0.01 mg/dL, p < 0.05; NGAL 4.53 ± 0.68 vs. 2.11 ± 0.33 mg/mL, p < 0.05), and the combined treatment of pFUS + EVs further reduced all these markers compared to treatment with EVs alone (BUN 50.74 ± 3.34 vs. 72.23 ± 5.61 mg/ dL, p < 0.05; SCr 0.61 ± 0.02 vs. 0.99 ± 0.01 mg/dL, p < 0.05; NGAL 1.70 ± 0.42 vs. 2.11 ± 0.33 mg/mL, p > 0.05).

### EV Homing to the Kidney is not Enhanced Following pFUS

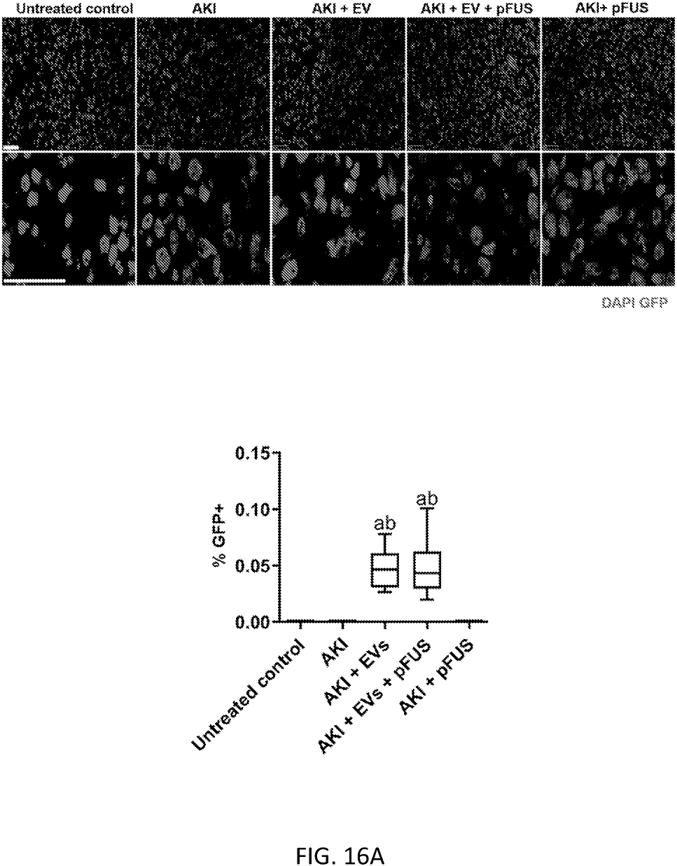

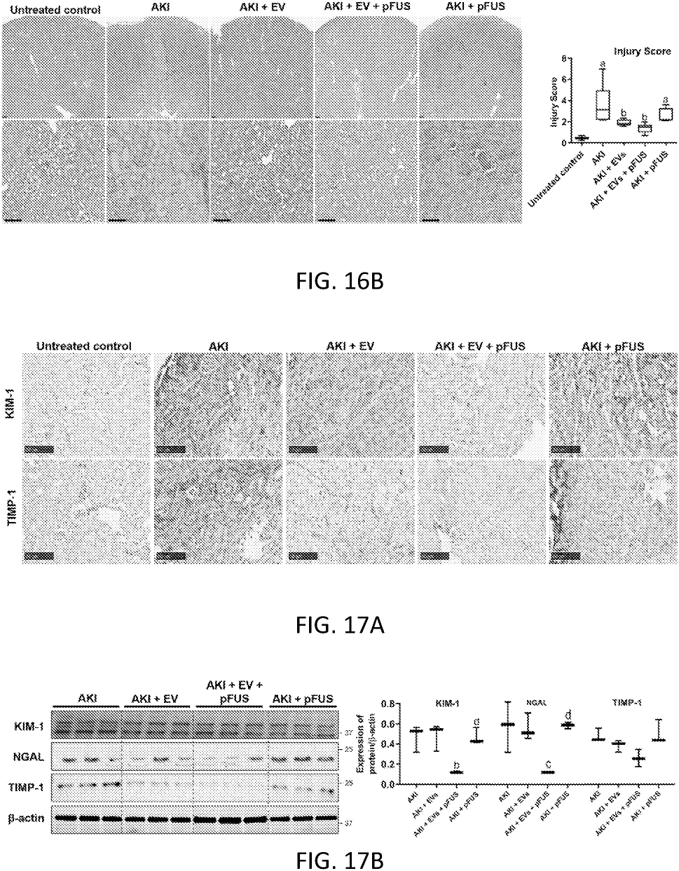

Following the development of AKI in mice, IV-administered EVs were able to home to the injured kidney, as demonstrated by the detection of GFP-labeled EVs within the kidney tissue at day 12 (FIG. 16A). However, pFUS had no additional effect on EV homing, as demonstrated by both histological detection and flow cytometry analysis (0.05 ± 0.02% vs. 0.05 ± 0.03% GFP positive, p > 0.05). As expected, no expression of GFP was detected in the kidneys of animals not treated with EVs.

### EVs and pFUS Reverse Histological and Molecular Markers of Kidney Damage

Histological evaluation of kidney tissue demonstrated that cisplatin induced a significant increase in injury compared to untreated controls, as demonstrated by the presence of morphological changes within the kidney parenchyma, including glomerular casts, tubular casts, and fibrosis (FIG. 16B). This injury was reflected in a higher histological injury score in the AKI group compared to untreated controls (3.65 ± 1.81 vs. 0.43 ± 0.16, p < 0.05). Compared to the AKI group, those treated with EVs alone showed a dramatically lower injury score (3.65 ± 1.81 vs. 1.90 ± 0.31, p < 0.05). pFUS alone also resulted in a lower injury score (3.65 ± 1.81 vs. 2.57 ± 0.66, p > 0.05) that did not reach statistical significance. Animals in the combined pFUS + EVs group had further reduced injury scores compared to those receiving either EVs alone (1.40 ± 0.45 vs. 1.90 ± 0.31, p > 0.05) or pFUS alone (1.40 ± 0.45 vs. 2.57 ± 0.66, p > 0.05), though these differences did not reach statistical significance (FIG. 16B).

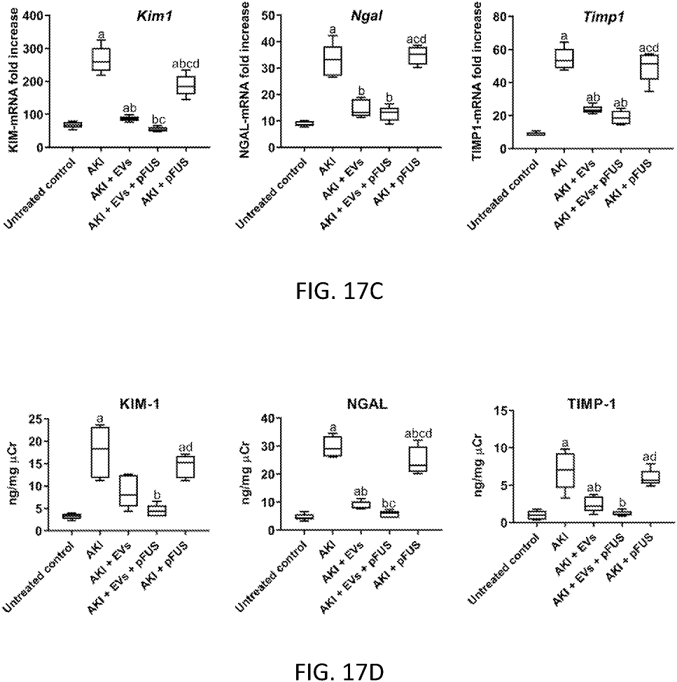

These trends were further confirmed by measuring the levels of tissue damage markers KIM-1 and TIMP-1. Immunohistochemical (IHC) staining of kidney tissue showed that untreated controls do not express these two molecules, but cisplatin-induced AKI strongly upregulated both markers (FIG. 17A). Although pFUS alone was not able to reduce their expression, EVs alone were able to partially reduce these two markers. Moreover, the combination of pFUS + EV treatment almost completely eliminated their presence (FIG. 17A). Western blot analysis further confirmed these trends: compared to the AKI group, EVs alone were able to partially reduce levels of KIM-1 (0.55 ± 0.16 vs. 0.38 ± 0.17 normalized expression, p > 0.05) and TIMP-1 (0.64 ± 0.40 vs. 0.38 ± 0.08 normalized expression, p > 0.05), but combined EVs + pFUS treatment showed much greater reductions in KIM-1 (0.55 ± 0.16 vs. 0.12 ± 0.02 normalized expression, p < 0.05), NGAL (0.44 ± 0.23 vs. 0.08 ± 0.02 normalized expression, p < 0.05), and TIMP-1 (0.64 ± 0.40 vs. 0.19 ±0.11 normalized expression, p > 0.05) (FIG. 17B). qRT-PCR analysis on mRNA expression in kidney tissue (FIG. 17C), as well as ELISA analysis on urine samples, further recapitulated these trends (FIG. 17D).

### EVs And pFUS Reduce Inflammatory Cytokines in the Setting of AKI

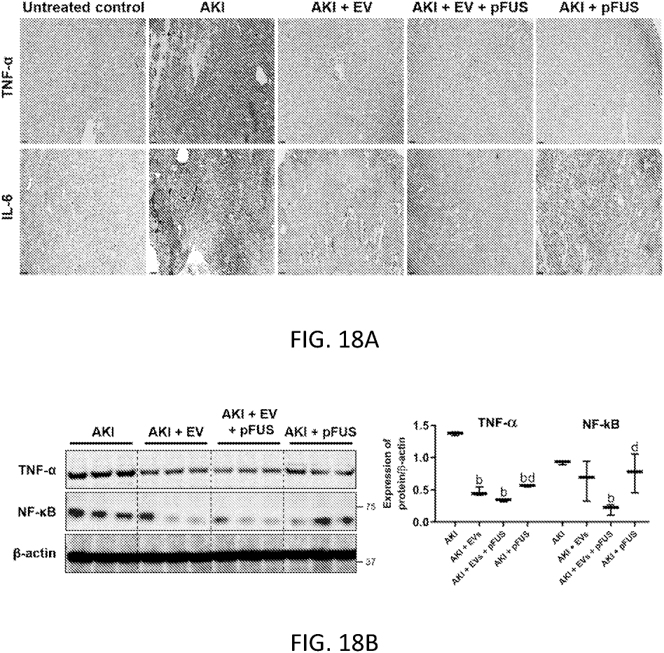

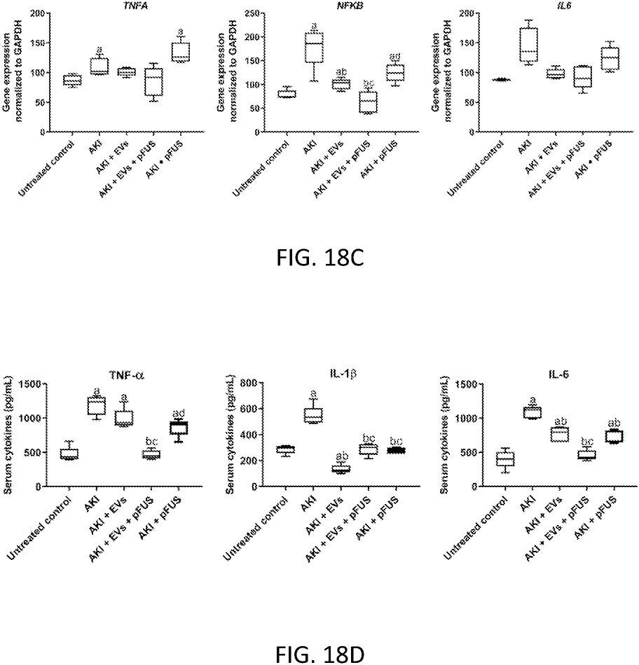

Having established the regenerative capabilities of EVs, we next sought to elucidate the biological mechanisms through which they reverse AKI-induced kidney damage. We found by IHC that cisplatin-induced AKI is characterized by high levels of inflammatory markers in the kidney, including TNF-α and IL-6 (FIG. 18A). Treatment with either EVs, pFUS, or the combined treatment dramatically reduced the expression of these cytokines. Western blot analysis further supported these trends. Combined pFUS + EV treatment reduced TNF-α more so than EVs alone (0.34 ± 0.02 vs. 0.47 ± 0.07 normalized expression, p > 0.05), as was the case with its downstream signaling protein NF-kB (0.20 ± 0.08 vs. 0.65 ± 0.31 normalized expression, p > 0.05), though these results did not reach statistical significance (FIG. 18B). Similar trends were observed with qRT-PCR analysis of kidney tissue mRNA: compared to untreated controls, cisplatin-induced AKI resulted in elevated transcript levels of Nfkb (78.88 ± 9.85 vs. 178.76 ± 41.93 relative expression, p < 0.05), IL6 (88.08 ± 1.65 vs. 144.56 ± 30.67 relative expression, p > 0.05), and TNFα (87.07 ± 9.06 vs. 108.95 ± 14.96 relative expression, p < 0.05) (FIG. 18C). Treatment with EVs, with or without pFUS, was able to bring transcript levels of these cytokines back to levels comparable to those of untreated controls. ELISA analysis also showed a significant reduction in serum cytokine levels when comparing the AKI group with those treated with pFUS + EVs, including TNF-α (1187 ± 142 vs. 463 ± 65 pg/mL, p < 0.05), IL-6 (1087 ± 90 vs. 454 ± 78 pg/mL, p < 0.05), and IL-1β (547 ± 75 vs. 290 ± 47 pg/mL, p < 0.05) (FIG. 18D). In fact, animals treated with the combined pFUS + EVs showed significantly more potent reduction in IL-6 and TNF-α compared to those treated with EVs alone (IL-6 454 ± 78 vs. 769 ± 109 pg/mL, p < 0.05; TNF-α 463 ± 65 vs. 987 ± 143 pg/mL, p < 0.05; IL-1β 290 ± 47 vs. 135 ± 33 pg/mL, p < 0.05).

### EVs and pFUS Promote Proliferation and Inhibit Apoptosis

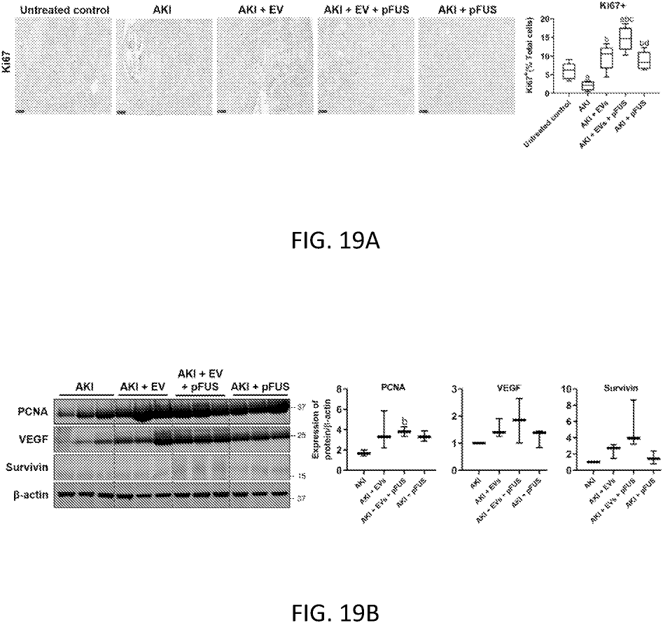

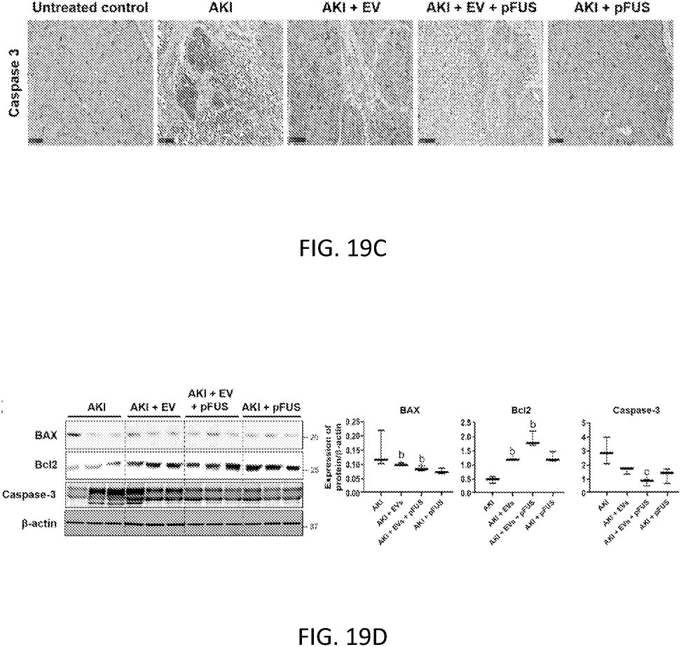

Modulation of cell proliferation and apoptosis may be another mechanism by which EVs may exert their therapeutic effect. Using IHC staining, we found that compared to untreated controls, the AKI group had dramatically reduced cell proliferation in the kidney, as measured by the proliferation marker Ki67 (6.21 ± 2.26% vs. 2.01 ± 1.21% Ki67⁺, p < 0.05) (FIG. 19A). Compared to the AKI group, the percentage of proliferating cells was restored after treatment with EVs alone (9.7 ± 3.35% vs. 2.01 ± 1.21% Ki67⁺, p < 0.05) or pFUS alone (8.77 ± 2.45% vs. 2.01 ± 1.21% Ki67⁺, p < 0.05), and the combined pFUS + EV treatment showed the greatest effect (14.67 ± 3.20% vs. 2.01 ± 1.21% Ki67⁺, p < 0.05), significantly more than either treatment alone. We also measured the expression of several proliferation markers by Western blot, including PCNA, VEGF, and survivin. EVs alone were able to somewhat increase PCNA, VEGF, and survivin expression compared to the AKI group, though the difference did not reach statistical significance. pFUS alone was only able to increase PCNA levels. Compared to the AKI group, the combination of pFUS + EVs was able to increase the levels of PCNA (1.68 ± 0.30 vs. 3.80 ± 0.47 normalized expression, p < 0.05), VEGF (1.02 ± 0.46 vs. 1.85 ± 0.25 normalized expression, p > 0.05), and survivin (0.23 ± 0.04 vs. 1.16 ± 0.44 normalized expression, p > 0.05), more so than either treatment alone (FIG. 19B).

In addition to proliferation, we also measured apoptosis markers (BAX, Bcl-2, Caspase-3) in the kidney tissue. IHC analysis showed that AKI strongly upregulated Caspase-3, indicative of significant apoptosis following injury (FIG. 19C). Both EVs alone and pFUS alone reduced the amount of Caspase-3, but the combined treatment of pFUS + EVs showed an even greater reduction. Western blot analysis further showed that EVs alone strongly upregulated the expression of anti-apoptotic proteins like Bcl-2 compared to the AKI group (1.18 ± 0.02 vs. 0.47 ± 0.11 normalized expression, p < 0.05) and that the combination of pFUS + EVs resulted in an even greater increase compared to EVs alone (1.88 ± 0.27 vs. 1.18 ± 0.02 normalized expression, p > 0.05) (FIG. 19D). On the other hand, compared to the AKI group, pro-apoptotic genes like BAX and Caspase-3 were downregulated by both EVs alone (BAX 0.10 ± 0.01 vs. 0.15 ± 0.06 normalized expression, p < 0.05; Caspase-3 1.63 ± 0.24 vs. 2.99 ± 0.95 normalized expression, p > 0.05) and by pFUS alone (BAX 0.07 ± 0.01 vs. 0.15 ± 0.06 normalized expression, p > 0.05; Caspase-3 1.29 ± 0.53 vs. 2.99 ± 0.94 normalized expression, p > 0.05). The combination of pFUS + EVs resulted in a significantly stronger downregulation of Caspase-3 than either treatment with EVs alone (0.80 ± 0.23 vs. 1.63 ± 0.24 normalized expression, p < 0.05) or pFUS alone (0.80 ± 0.23 vs. 1.29 ± 0.53 normalized expression, p > 0.05).

### EVs And pFUS Synergistically Activate MAPK and Akt Signaling

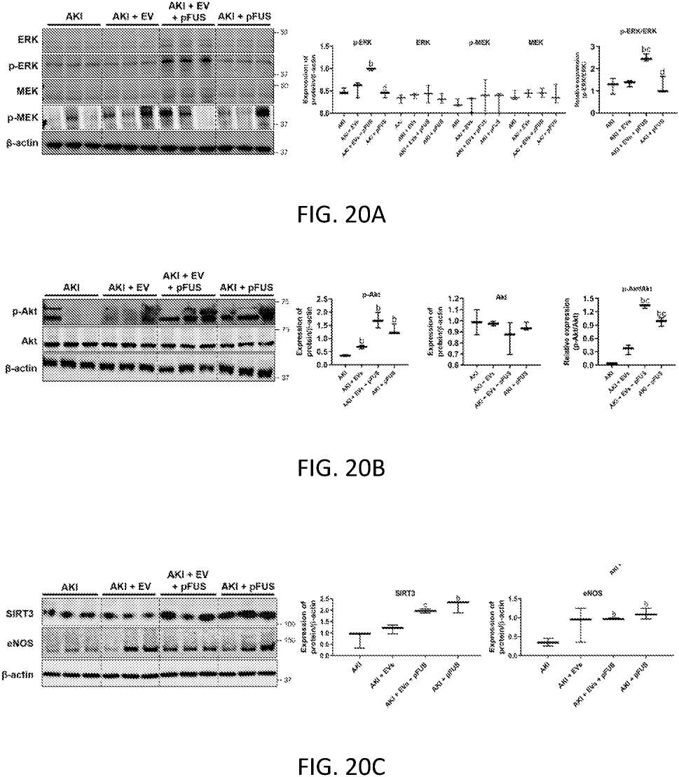

We next sought to identify the molecular signaling pathways used by EVs to exert their regenerative properties. Western blot analysis of kidney tissue showed that compared to the AKI group, combined treatment with pFUS + EVs significantly upregulated MAPK/ERK signaling, as measured by the relative expression of phosphorylated to total ERK (1.23 ± 0.35 vs. 2.49 ± 0.16, p < 0.05) (FIG. 20A). However, no differences were found in the levels of phosphorylated or total MEK across the experimental groups. We also measured PI3K/Akt signaling based on the relative expression of phosphorylated to total Akt. Compared to the AKI group, PI3K/Akt signaling was upregulated by EVs alone (0.34 ± 0.03 vs. 0.68 ± 0.07, p < 0.05) and by pFUS alone (0.34 ± 0.03 vs. 1.32 ± 0.21, p < 0.05), with the combined treatment of pFUS + EVs resulting in an even greater upregulation (0.34 ± 0.03 vs. 1.69 ± 0.29, p < 0.05) (FIG. 20B). Finally, we measured the protein expression of SIRT3 and eNOS, two markers of regeneration and angiogenesis. Compared to the AKI group, SIRT3 was modestly upregulated by EVs alone (0.76 ± 0.37 vs. 1.18 ± 0.19 normalized expression, p > 0.05) and significantly upregulated by pFUS alone (0.76 ± 0.37 vs. 2.19 ± 0.27 normalized expression, p < 0.05). pFUS + EVs resulted in significantly higher upregulation of SIRT3 compared to EVs alone (1.96 ± 0.09 vs. 1.18 ± 0.19 normalized expression, p < 0.05). Compared to the AKI group, eNOS was upregulated in all three treatment groups: EVs alone (0.35 ± 0.11 vs. 0.97 ± 0.02 normalized expression, p < 0.05), pFUS alone (0.35 ± 0.11 vs. 1.12 ± 0.14 normalized expression, p < 0.05), and pFUS + EVs (0.35 ± 0.11 vs. 0.85 ± 0.46 normalized expression, p > 0.05) (FIG. 20C). However, in this case, the combined pFUS + EV treatment was not superior to the individual treatments.

### Discussion

In this study, we demonstrated that EVs are capable of reversing cisplatin-induced AKI and that this regenerative effect is enhanced when they are used in combination with pFUS. We have demonstrated several mechanisms by which EVs and pFUS exert a synergistic therapeutic effect, including (1) a reduction in inflammatory cytokines, (2) an increase in cell proliferation, and (3) a decrease in apoptosis. These effects are mediated by increased MAPK/ERK and PI3K/Akt signaling and upregulation of the SIRT3 and eNOS pathways.

pFUS presents a promising method for optimizing MSC-based therapies [28]. It is a non-invasive procedure that can be precisely targeted to deep structures in the body and has an excellent safety profile [32, 34]; indeed, focused ultrasound is already FDA-approved for various clinical applications [41]. Burks et al. have previously tested pFUS in the setting of cisplatin-induced AKI, though there are some discrepancies between their studies and ours [30]. In AKI, they found that pFUS alone did not significantly improve kidney function (creatinine and BUN), promote cell proliferation (Ki67 and p-Akt), or reduce apoptosis and necrosis. However, pFUS alone did elicit these effects in our current and previous experiments [29, 42]. This discrepancy may result from different ultrasound transducers and settings; given that there is a lack of data on how the biological effects of pFUS are influenced by specific ultrasound parameters, direct comparison between studies is difficult.

These discrepancies suggest that there are different molecular mechanisms by which pFUS operates, depending on ultrasound parameters. The studies by Burks et al. show that pFUS transiently activates TNF-α and IL-1 α expression in the sonicated tissue, which act through NF-kB signaling to drive cyclooxygenase-2 (COX2) activity; in turn, this promotes the expression of several homing factors and increases the local accumulation of MSCs, thus augmenting their therapeutic effect [43, 44]. Our previous studies have shown, however, that pFUS can improve MSC therapy independently of improved homing [29]. Instead, pFUS modulates the expression of heat shock proteins (HSPs), leading to various downstream effects such as increased proliferation through PI3K/Akt signaling [29] and suppression of inflammation [42]. In line with these previously reported mechanisms, the current study has identified the involvement of MAPK/ERK and PI3K/Akt signaling, canonical pathways for cell proliferation and survival that play a role in facilitating regeneration of damaged tissue [45, 46]. Our study also implicates the SIRT3 and eNOS pathways. SIRT3 is a mitochondrial protein deacetylase known for its ability to regulate energy demand during stressful conditions, eliminate reactive oxygen species, and prevent apoptosis [47]. eNOS, the endothelial nitric oxide synthase, has been shown to play an important role in neovascularization [48]. The involvement of these two pathways further suggests that EVs and pFUS can induce angiogenesis and stimulate regeneration.

One notable result of our study is that pFUS did not increase EV homing to the kidney. Homing is not a passive process, but an active one that requires a sequential series of molecular interactions. The homing process for MSCs is well-characterized: in order to travel from circulation into a target tissue, they must undergo (1) tethering by selectins, (2) activation by chemokines, (3) arrest by integrins, (4) transmigration or diapedesis across the endothelial layer, and (5) extravascular migration to the target tissue mediated by cytokines [49]. pFUS at certain parameters can enhance this process by upregulating homing molecules at the target tissue, especially chemokines like SDF-1 and cell adhesion molecules like VCAM-1 [31-34]. Many studies have demonstrated increased MSC homing following pFUS [30, 32, 34, 35], but whether the same effect would be true of EVs was unknown. In our study, pFUS did not enhance EV homing. One possible explanation may lie in different pFUS parameters: different sonication intensities are known to elicit distinct biological effects, only some of which may generate the cytokine gradient responsible for enhanced homing [28, 50]. Another possible explanation is that the biology of EVs may not permit the same mechanisms of homing as their parent MSCs. In order for EVs to show enhanced homing following pFUS, they must express the relevant homing factors on their cell surface. EVs include particles that are generated within endosomal compartments and released when their surrounding compartment fuses with the cell membrane [26]. Hence, EVs would not necessarily have the same surface markers as their parent MSCs. However, they still express surface markers relevant for homing [51], such as the selectin receptor CD44 [52, 53] which facilitates initial tethering and rolling adhesion [54], and various integrins which facilitate homing to specific tissues [52, 53, 55]. More systematic efforts to characterize EV surface proteins would be valuable for assessing the feasibility of improving EV homing and reveal possible strategies for doing so. To our knowledge, there has only been one other study on the effect of pFUS on EV homing, though in a rather different setting. Bai et al. reported that pFUS increases the homing of blood serum-derived EVs to the brain by 4.5-fold [56]. However, several reasons may explain the discrepancy with our results. First, their EVs are derived from different sources and thus may express different homing factors. Second, the increased homing is likely more the result of blood-brain barrier opening, a known effect of pFUS on the brain [57], not true improvement of the molecular homing process. Finally, different ultrasound parameters could have elicited different biological effects between our studies.

Most previous studies attribute the enhanced therapeutic outcomes following pFUS to increased MSC homing. However, our results suggest there are other different mechanisms by which pFUS independently facilitates tissue regeneration. pFUS on its own seems to exert biological effects sufficient for tissue regeneration. Based on our data, pFUS alone was able to reduce inflammatory cytokines, induce PI3K/Akt signaling with subsequent increases in proliferating cells and decreased apoptosis, and upregulate SIRT3 and eNOS. Consistent with these findings, a previous study has shown that ultrasound can prevent AKI by stimulating cholinergic anti-inflammatory pathways [58], though that study used unfocused ultrasound. These effects of pFUS alone were observed in some previous studies [29, 42], but not others [30], which may be an effect of different ultrasound parameters. It is also possible that there may be some sensitization effect, wherein pFUS sensitizes the target tissue to the regenerative molecules carried by EVs, thereby increasing their therapeutic efficacy; this hypothesis, though exciting, requires further exploration.

### Conclusions

Our study demonstrates that the combination of pFUS + EVs is therapeutic in the setting of cisplatin-induced AKI, superior to either EVs or pFUS alone. AKI represents a growing clinical concern for which there are no curative therapies. Though there has been considerable preclinical success in using EVs therapeutically, more optimization remains to be done before they may reach clinical utility. EVs are an attractive cell-free alternative to MSC therapy. Their small size bypasses the pulmonary first-pass effect, where the vast majority of IV-infused MSCs become entrapped in the lung microvasculature [19-23]. EVs also obviate the need for a constant supply of cells and avoid concerns regarding unwanted engraftment or immune rejection [59]. Rigorous molecular characterization of the mechanisms underlying EV and pFUS therapy, alongside a better understanding of pFUS parameters, will be key to fully realizing their clinical potential.

### References

1. Levey AS, James MT. Acute kidney injury. Ann Intern Med. 2017;167(9): ITC66-80.

2. Pavkov ME, Harding JL, Burrows NR. Trends in hospitalizations for acute kidney injury-United States, 2000-2014. MMWR Morb Mortal Wkly Rep. 2018;67(10):289-93.

3. Kolhe NV, et al. National trends in acute kidney injury requiring dialysis in England between 1998 and 2013. Kidney Int. 2015;88(5):1161-9.

4. Carlson N, et al. Trends in one-year outcomes of dialysis-requiring acute kidney injury in Denmark 2005-2012: a population-based nationwide study. PLoS One. 2016;11(7):e0159944.

5. Coca SG, Singanamala S, Parikh CR. Chronic kidney disease after acute kidney injury: a systematic review and meta-analysis. Kidney Int. 2012;81(5):442-8.

6. Chawla LS, Kimmel PL. Acute kidney injury and chronic kidney disease: an integrated clinical syndrome. Kidney Int. 2012;82(5):516-24.

7. Chawla LS, et al. Acute kidney injury and chronic kidney disease as interconnected syndromes. N Engl J Med. 2014;371 (1 ):58-66.

8. Pannu N. Bidirectional relationships between acute kidney injury and chronic kidney disease. Curr Opin Nephrol Hypertens. 2013;22(3):351-6.

9. Coca SG, Cho KC, Hsu CY. Acute kidney injury in the elderly: predisposition to chronic kidney disease and vice versa. Nephron Clin Pract. 2011; 119(Suppl 1):c19-24.

10. Hsu RK, Hsu CY. The role of acute kidney injury in chronic kidney disease. Semin Nephrol. 2016;36(4):283-92.

11. Mo M, et al. Mesenchymal stem cell subpopulations: phenotype, property and therapeutic potential. Cell Mol Life Sci. 2016;73(17):3311-21.

12. Fan XL, et al. Mechanisms underlying the protective effects of mesenchymal stem cell-based therapy. Cell Mol Life Sci. 2020;77(14):2771-94.

13. Murphy MB, Moncivais K, Caplan Al. Mesenchymal stem cells: environmentally responsive therapeutics for regenerative medicine. Exp Mol Med. 2013;45:e54.

14. Herrera MB, et al. Mesenchymal stem cells contribute to the renal repair of acute tubular epithelial injury. Int J Mol Med. 2004;14(6):1035-41.

15. Morigi M, et al. Mesenchymal stem cells are renotropic, helping to repair the kidney and improve function in acute renal failure. J Am Soc Nephrol. 2004;15(7):1794-804.

16. Caplan Al. Why are MSCs therapeutic? New data: new insight. J Pathol. 2009;217(2):318-24.

17. Morigi M, De Coppi P. Cell therapy for kidney injury: different options and mechanisms--mesenchymal and amniotic fluid stem cells. Nephron Exp Nephrol. 2014;126(2):59.

18. Lai RC, Yeo RW, Lim SK. Mesenchymal stem cell exosomes. Semin Cell Dev Biol. 2015;40:82-8.

19. Schrepfer S, et al. Stem cell transplantation: the lung barrier. Transplant Proc. 2007;39(2):573-6.

20. Santeramo I, et al. Human kidney-derived cells ameliorate acute kidney injury without engrafting into renal tissue. Stem Cells Transl Med. 2017;6(5): 1373-84.

21. Leibacher J, Henschler R. Biodistribution, migration and homing of systemically applied mesenchymal stem/stromal cells. Stem Cell Res Ther. 2016;7:7.

22. Eggenhofer E, et al. Mesenchymal stem cells are short-lived and do not migrate beyond the lungs after intravenous infusion. Front Immunol. 2012;3:297.

23. Fischer UM, et al. Pulmonary passage is a major obstacle for intravenous stem cell delivery: the pulmonary first-pass effect. Stem Cells Dev. 2009; 18(5):683-92.

24. Lv LL, et al. Therapeutic application of extracellular vesicles in kidney disease: promises and challenges. J Cell Mol Med. 2018;22(2):728-37.

25. Phinney DG, Pittenger MF. Concise review: MSC-derived exosomes for cell-free therapy. Stem Cells. 2017;35(4):851-8.

26. Cheng L, et al. Focus on mesenchymal stem cell-derived exosomes: opportunities and challenges in cell-free therapy. Stem Cells Int. 2017;2017:6305295.

27. Ullah M, et al. A novel approach to deliver therapeutic extracellular vesicles directly into the mouse kidney via its arterial blood supply. Cells. 2020;9(4): 937.

28. Liu DD, et al. The role of ultrasound in enhancing mesenchymal stromal cell-based therapies. Stem Cells Transl Med, 2020;9(8):850-66.

29. Ullah M, et al. Reversing acute kidney injury using pulsed focused ultrasound and MSC therapy: a role for HSP-mediated PI3K/AKT signaling. Mol Ther Methods Clin Dev. 2020;17:683-94.

30. Burks SR, et al. Pulsed focused ultrasound pretreatment improves mesenchymal stromal cell efficacy in preventing and rescuing established acute kidney injury in mice. Stem Cells. 2015;33(4):1241-53.

31. Burks SR, et al. Investigation of cellular and molecular responses to pulsed focused ultrasound in a mouse model. PLoS One. 2011;6(9):e24730.

32. Ziadloo A, et al. Enhanced homing permeability and retention of bone marrow stromal cells by noninvasive pulsed focused ultrasound. Stem Cells. 2012;30(6):1216-27.

33. Jang KW, et al. Molecular and histological effects of MR-guided pulsed focused ultrasound to the rat heart. J Transl Med. 2017;15(1):252.

34. Burks SR, et al. Noninvasive pulsed focused ultrasound allows spatiotemporal control of targeted homing for multiple stem cell types in murine skeletal muscle and the magnitude of cell homing can be increased through repeated applications. Stem Cells. 2013;31(11):2551-60.

35. Tebebi PA, et al. Improving the therapeutic efficacy of mesenchymal stromal cells to restore perfusion in critical limb ischemia through pulsed focused ultrasound. Sci Rep. 2017;7:41550.

36. Burks SR, et al. Mesenchymal stromal cell potency to treat acute kidney injury increased by ultrasound-activated interferon-gamma/interleukin-10 axis. J Cell Mol Med. 2018;22(12):6015-25.

37. Kim DK, et al. Chromatographically isolated CD63+CD81+ extracellular vesicles from mesenchymal stromal cells rescue cognitive impairments after TBI. Proc Natl Acad Sci USA. 2016;113(1):170-5.

38. Bartosh TJ, et al. Cancer cells enter dormancy after cannibalizing mesenchymal stem/stromal cells (MSCs). Proc Natl Acad Sci U S A. 2016;113(42):E6447-56.

39. Klopfleisch R. Multiparametric and semiquantitative scoring systems for the evaluation of mouse model histopathology--a systematic review. BMC Vet Res. 2013;9:123.

40. Pfaffl MW. A new mathematical model for relative quantification in real-time RT-PCR. Nucleic Acids Res. 2001;29(9):e45.

41. Tempany CM, et al. Focused ultrasound surgery in oncology: overview and principles. Radiology. 2011;259(1):39-56.

42. Ullah M, et al. HSP70-mediated NLRP3 inflammasome suppression underlies reversal of acute kidney injury following extracellular vesicle and focused ultrasound combination therapy. IntJ Mol Sci. 2020;21(11):4085.

43. Tebebi PA, et al. Cyclooxygenase-2 or tumor necrosis factor-alpha inhibitors attenuate the mechanotransductive effects of pulsed focused ultrasound to suppress mesenchymal stromal cell homing to healthy and dystrophic muscle. Stem Cells. 2015;33(4):1173-86.

44. Burks SR, et al. Anti-inflammatory drugs suppress ultrasound-mediated mesenchymal stromal cell tropism to kidneys. Sci Rep. 2017;7(1):8607.

45. Zhang W, Liu HT. MAPK signal pathways in the regulation of cell proliferation in mammalian cells. Cell Res. 2002;12(1):9-18.

46. Manning BD, Toker A. AKT/PKB signaling: navigating the network. Cell. 2017; 169(3):381-405.

47. Ansari A, et al. Function of the SIRT3 mitochondrial deacetylase in cellular physiology, cancer, and neurodegenerative disease. Aging Cell. 2017;16(1):4-16.

48. Bir SC, et al. Emerging role of PKA/eNOS pathway in therapeutic angiogenesis for ischaemic tissue diseases. Cardiovasc Res. 2012; 95(1):7-18.

49. Ullah M, Liu DD, Thakor AS. Mesenchymal stromal cell homing: mechanisms and strategies for improvement. iScience. 2019;15:421-38.

50. Razavi M, et al. Effect of pulsed focused ultrasound on the native pancreas. Ultrasound Med Biol. 2019;46(3):630-8.

51. Anderson JD, et al. Comprehensive proteomic analysis of mesenchymal stem cell exosomes reveals modulation of angiogenesis via nuclear factor-kappaB signaling. Stem Cells. 2016;34(3):601-13.

52. Choi HY, et al. Microparticles from kidney-derived mesenchymal stem cells act as carriers of proangiogenic signals and contribute to recovery from acute kidney injury. PLoS One. 2014;9(2):e87853.

53. Bruno S, et al. Mesenchymal stem cell-derived microvesicles protect against acute tubular injury. J Am Soc Nephrol. 2009;20(5): 1053-67.

54. Sackstein R, et al. Ex vivo glycan engineering of CD44 programs human multipotent mesenchymal stromal cell trafficking to bone. Nat Med. 2008; 14(2):181-7.

55. Shimaoka M, et al. Connexins and integrins in exosomes. Cancers (Basel). 2019;11(1):106.

56. Bai L, et al. Ultrasound facilitates naturally equipped exosomes derived from macrophages and blood serum for orthotopic glioma treatment. ACS Appl Mater Interfaces. 2019;11(16):14576-87.

57. Kovacs Zl, et al. Disrupting the blood-brain barrier by focused ultrasound induces sterile inflammation. Proc Natl Acad Sci U S A. 2017;1 14(1):E75-84.

58. Gigliotti JC, et al. Ultrasound prevents renal ischemia-reperfusion injury by stimulating the splenic cholinergic anti-inflammatory pathway. J Am Soc Nephrol. 2013;24(9):1451-60.

59. Lou G, et al. Mesenchymal stem cell-derived exosomes as a new therapeutic strategy for liver diseases. Exp Mol Med. 2017;49(6):e346.

### Example 4 HSP70-Mediated NLRP3 Inflammasome Suppression Underlies Reversal of Acute Kidney Injury Following Extracellular Vesicle and Focused Ultrasound Combination Therapy

### Introduction

Acute kidney injury (AKI) is the sudden loss of renal function, usually due to ischemia, nephrotoxic agents, or urinary tract obstructions [1]. Although AKI is a relatively common condition, especially in hospitalized and chronically ill patients, treatments remain largely supportive, despite mortality associated with this condition being as high as 20% [2]. Hence, there is growing interest in developing regenerative therapies for AKI that can repair renal injury as well as prevent its progression to chronic kidney disease.

AKI is associated with both systemic and intrarenal inflammation, which are believed to be key components underlying its pathophysiology [3]. Although inflammation in the acute phase can facilitate tissue repair following injury, disruption of this process can lead to persistent inflammation, causing tissue damage and fibrosis [4]. Many molecular mediators of inflammation have been identified in AKI [5], which include the NLRP3 inflammasome [6], toll-like receptors (TLRs) [7], and various secreted cytokines that promote neutrophil- and monocyte-mediated inflammatory responses [5,8]. Indeed, blockade of innate immune receptors seems to confer protection against AKI in several preclinical studies [9-12].

Another therapeutic strategy for immune modulation lies in mesenchymal stromal cell (MSC)-based therapies [13]. MSCs are multipotent cells that have been investigated as a cell therapy for regenerative medicine applications, including AKI [14,15]. Their therapeutic effect arises from their ability to home to damaged tissue and secrete extracellular vesicles (EVs) and other factors that act in a paracrine manner to exert proliferative, pro-survival, and anti-inflammatory effects [16-19]. More recent studies have begun exploring purified MSC-derived EVs as a cell-free alternative to MSC therapy [20-22]; the advantages of using EVs compared to MSCs include their higher safety profile, ability to cross barriers with minimal sequestration in the pulmonary microvasculature following intravenous infusion, lower immunogenicity, and avoidance of complications related to stem cell-induced tumor formation [23-28].

While EVs can achieve a therapeutic effect comparable to their parent MSCs in the context of AKI, there remains great interest in optimizing their efficacy. Pulsed focused ultrasound (pFUS), where target organs are selectively treated with focused sound waves, has recently emerged as a method to improve MSC-based therapies [29]. Pre-treatment of target organs with pFUS has been shown to locally upregulate cytokines and trophic factors, improve MSC homing, and subsequently their therapeutic efficacy [30-32]. However, the full range of mechanisms underlying pFUS has yet to be elucidated [29,33], and its effect on EV therapy is particularly lacking. Here, we assess the effect of combination therapy with pFUS and MSC-derived EVs in a mouse model of cisplatin-induced AKI, evaluating in particular their ability to suppress AKI-related inflammation.

### 2. Results

### 2.1. Reversal of AKI Using EVs and pFUS

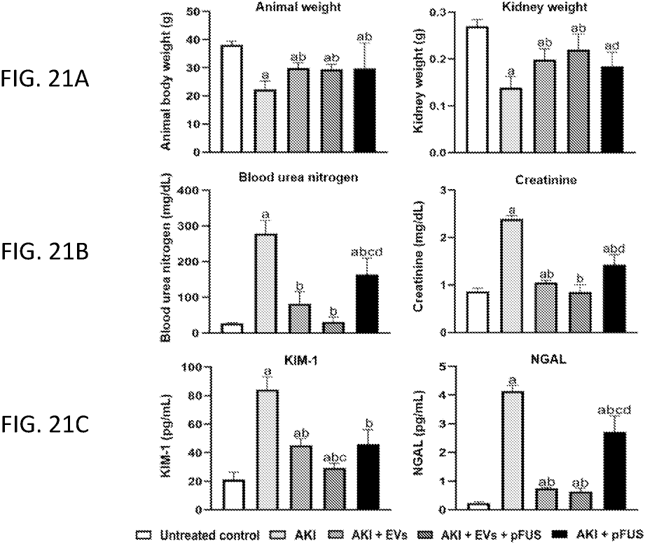

Following the induction of AKI using cisplatin at day zero, mice were either administered pFUS at day 2, EVs at day 3, or a combination of both. Compared to untreated controls, mice with AKI showed a significant decrease in the animal body weight (38.14 ± 1.35 vs. 22.29 ± 3.04 g, p < 0.05) and kidney weight (0.27 ±0.01 vs. 0.14 ±0.02 g, p < 0.05) (FIG. 21 A), significant increases in blood urea nitrogen (BUN) (27.27 ± 1.52 vs. 278.48 + 37.29 mg/dL, p < 0.05) and serum creatinine (SCr) (0.86 ±0.07 mg/dL vs. 2.39 ±0.07, p < 0.05) (FIG. 21 B), and significant increases in serum concentration of molecular injury markers KIM-1 (21.17 ±5.17 vs. 84.19 ±8.96 pg/mL, p < 0.05) and NGAL (0.23 ±0.04 vs. 4.14 ±0.20 pg/mL, p < 0.05) (FIG. 21C).

Compared to mice in the AKI group, those treated with pFUS alone demonstrated a significant increase in body weight (22.29 ±3.04 vs. 29.71 ±9.15 g, p < 0.05) and non-significant increase in kidney weight (0.14 ± 0.02 vs. 0.18 + 0.03 g, p > 0.05) (FIG. 21A), a significant decrease in BUN (278.48 ± 37.29 vs. 164.33 ± 45.74 mg/dL, p < 0.05) and SCr (2.39 ± 0.07 vs. 1.43 ±0.21 mg/dL, p < 0.05) (FIG. 21B), and a significant decrease in serum KIM-1 (84.19 ±8.96 vs. 45.95 ± 10.28 pg/mL, p < 0.05) and NGAL (4.14 +0.20 vs. 2.71 +0.56 pg/mL, p < 0.05) (FIG. 21C).

Compared to mice in the AKI group, those treated with EVs alone also demonstrated a significant increase in body weight (22.29 ± 3.04 vs. 29.85 ± 1.95 g, p < 0.05) and kidney weight (0.14 ±0.02 vs. 0.20 ±0.02 g, p < 0.05) (FIG. 21A), a significant decrease in BUN (278.48 ± 37.29 vs. 81.89 ± 34.11 mg/dL, p < 0.05) and SCr (2.39 ± 0.07 vs. 1.05 ± 0.05 mg/dL, p < 0.05) (FIG. 21B), and a significant decrease in serum KIM-1 (84.19 ±8.96 pg/mL vs. 45.18 ±4.71, p < 0.05) and NGAL (4.14 ±0.20 pg/mL vs. 0.64 ±0.11, p < 0.05) (FIG. 21C). Overall, the effect of EVs alone was more pronounced on than that of pFUS alone.

Notably, combined treatment with EVs and pFUS often had a greater effect than either alone. Compared to those treated with EVs alone, mice treated with pFUS + EVs had higher kidney weight (0.20 ±0.02 vs. 0.22 ±0.03 g, p > 0.05) (FIG. 21A), lower BUN (81.89 ±34.11 vs. 31.00 ±13.61 mg/dL, p >0.05) and SCr (1.05 ± 0.05 vs. 0.85 ± 0.15 mg/dL, p >0.05) (FIG. 21B), and lower serum levels of KIM-1 (45.18 ± 4.71 vs. 29.36 ± 3.29 pg/mL, p < 0.05) and NGAL (0.75 ± 0.03 vs. 0.64 ± 0.11, p > 0.05) (FIG. 21C). Though these differences often did not reach statistical significance there was a consistent trend for all measured physiological and molecular markers.

### 2.2. HSP70-Mediated Regulation of the NLRP3 Inflammasome

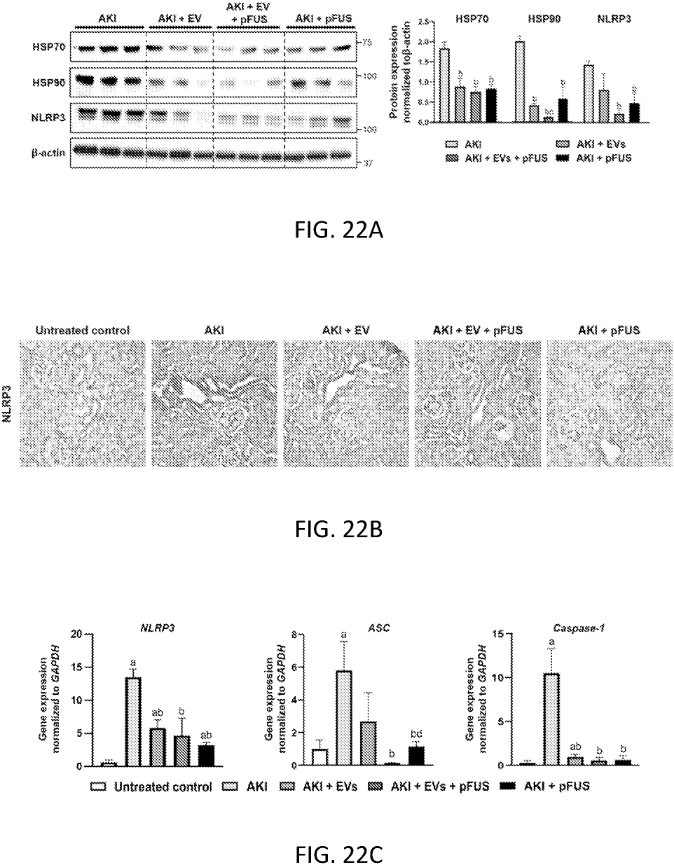

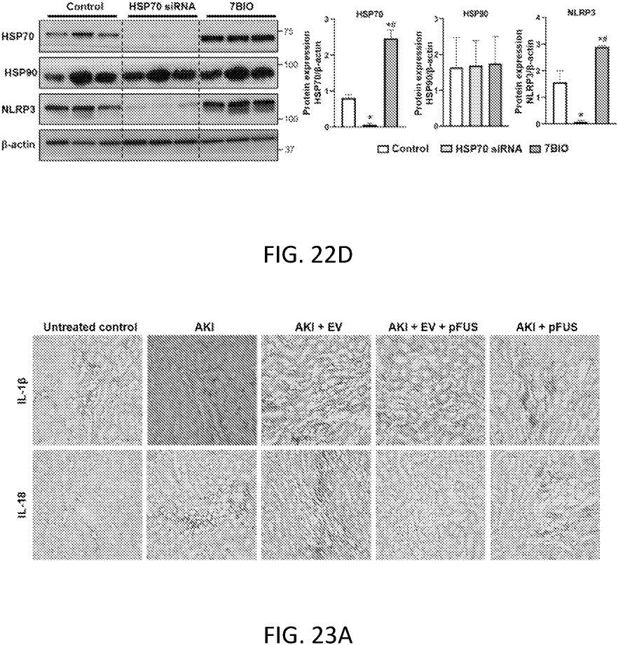

Through Western blot analysis, we found that the heat shock proteins HSP70 and HSP90 are strongly downregulated in the kidneys by both EVs alone (HSP70: 0.89 ± 0.21 vs. 1.23 ± 0.16 normalized expression, p < 0.05; HSP90: 0.41 ± 0.04 vs. 1.99 ± 0.12 normalized expression, p < 0.05) and by pFUS alone (HSP70: 0.84 ± 0.11 vs. 1.23 ± 0.16 normalized expression, p < 0.05; HSP90: 0.58 ± 0.28 vs. 1.99 ± 0.12 normalized expression, p < 0.05) compared to the AKI group (FIG. 22A). The combined pFUS + EVs treatment downregulated these proteins more so than EVs alone (HSP70: 0.75 ± 0.13 vs. 0.89 ± 0.21 normalized expression, p > 0.05; HSP90: 0.12 ± 0.01 vs. 0.41 ±0.40 normalized expression, p < 0.05).

As HSPs are known to regulate inflammation [34], we assessed whether there was any correlation between the expression of HSP70/90 and inflammasome proteins. Compared to the AKI group, NLRP3 was suppressed in the kidneys by both EVs alone (1.42 ± 0.10 vs. 0.81 ± 0.41 normalized expression, p > 0.05) and pFUS alone (1.42 ± 0.10 vs. 0.47 ± 0.26 normalized expression, p < 0.05) (FIG. 22A). The combined pFUS + EV treatment, however, resulted in the most potent suppression of NLRP3 compared the AKI group (1.42 ± 0.10 vs. 0.21 ± 0.09 normalized expression, p < 0.05). IHC staining for NLRP3 on kidney tissue recapitulated these findings, with the strongest NLRP3 staining in the AKI condition and markedly reduced staining following treatment with EVs, pFUS, or both (FIG. 22B). Similar trends were observed using qRT-PCR analysis of NLRP3 in kidney lysate, as well as two other inflammasome components, apoptosis-associated speck-like protein containing a CARD (ASC) and Caspase-1 (FIG. 22C). Compared to untreated controls, those in the AKI group had significant elevations of ASC and Caspase-1 in the kidney (ASC: 1.00 ± 0.56 vs. 5.80 ± 1.78 normalized expression, p < 0.05; Caspase-1: 0.29 +0.24 vs. 10.49 ± 2.82 normalized expression, p < 0.05). Compared to the AKI group, expression of these genes was suppressed following treatment with either EVs alone (ASC: 5.80 ± 1.78 vs. 2.69 ±1.74 normalized expression, p > 0.05; Caspase-1: 10.49 ± 2.82 vs. 0.97 ±0.29 normalized expression, p < 0.05) or pFUS alone (ASC: 5.80 ± 1.78 vs. 1.15 ±0.30 normalized expression, p < 0.05; Caspase-1: 10.49 ± 2.82 vs. 0.60 ± 0.49 normalized expression, p < 0.05). Combined treatment with pFUS + EVs resulted in an even greater suppression of these genes compared to EVs alone (ASC: 0.14 ± 0.03 vs. 2.69 ±1.74 normalized expression, p > 0.05; Caspase-1: 0.57 ±0.31 vs. 0.96 +0.29 normalized expression, p > 0.05), though the differences did not reach statistical significance.

We next sought to validate the mechanistic link between HSPs and the NLRP3 inflammasome. HSP90 has already been documented to positively regulate the NLRP3 inflammasome [35-37], but HSP70 has a less straightforward role [38-40], leading us to further investigate the latter. We treated human embryonic kidney (HEK) cells with either an HSP70 siRNA or with 7BIO (a non-apoptotic cell death inducer). Knockdown of HSP70 suppressed expression of the inflammasome protein NLRP3, while administration of 7BIO upregulated both HSP70 and NLRP3 (FIG. 22D), suggesting that HSP70 is a positive regulator of the NLRP3 inflammasome. Neither condition affected HSP90 expression.

### 2.3. Suppression of Inflammation Following EV and pFUS Therapy

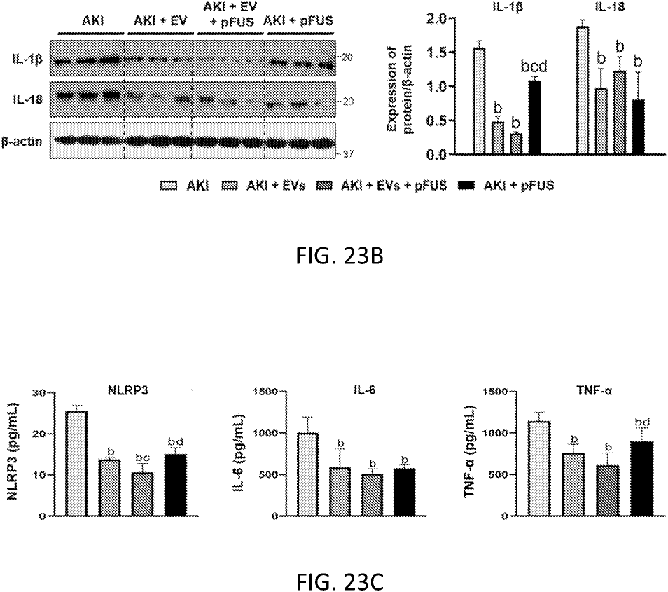

Given that pFUS and EVs suppress the NLRP3 inflammasome, we next assessed the extent of systemic and intrarenal inflammation following treatment. Using IHC, we found that IL-1β and IL-18, two pro-inflammatory cytokines activated by the NLRP3 inflammasome, were significantly increased in the AKI condition, while treatment with EVs, pFUS, or both reduced their expression (FIG. 23A). These results were confirmed by Western blot: compared to the AKI group, IL-1β and IL-18 were suppressed by both EVs alone (IL-1β: 1.57 ± 0.10 vs. 0.48 ± 0.07 relative expression, p < 0.05; IL-18: 1.88 ±0.10 vs. 0.98 ±0.28 relative expression, p < 0.05) and pFUS alone (IL-1β: 1.57 ±0.10 vs. 1.08 +0.07 relative expression, p <0.05; IL-18: 1.88 +0.10 vs. 0.81 ± 0.40 relative expression, p < 0.05) (FIG. 23B). The combined pFUS + EVs treatment resulted in more potent suppression of IL-1β than either EVs alone (0.48 ± 0.07 vs. 0.31 ± 0.01 relative expression, p > 0.05) or pFUS alone (1.08 ± 0.07 vs. 0.31 ± 0.01 relative expression, p < 0.05). Similar trends were observed using serum ELISA of NLRP3, IL-6, and TNF-α, with significant decreases following treatment with either EVs alone or pFUS alone, and the most potent decreases following combination therapy with pFUS + EVs (FIG. 23C).

### 3. Discussion

We have shown that the pretreatment of kidneys suffering from AKI with pFUS enhances the therapeutic effect of MSC-derived EVs. This synergistic effect is at least in part due to downregulation of HSP70, which in turn reduces the formation of the NLRP3 inflammasome, resulting in the attenuation of the pro-inflammatory environment characteristic of AKI (FIG. 4).

pFUS is a non-invasive procedure with an excellent safety profile that can be precisely targeted to deep body tissues, and is already FDA-approved for several clinical applications [41]. pFUS has previously been shown to enhance MSC therapy for AKI. However, the mechanism by which this occurs has yet to be fully understood, likely due to differences in ultrasound parameters used between various groups [29]. Some studies have reported that pFUS upregulates local cytokines which serve as a homing signal for MSCs, thereby increasing their accumulation in sonicated tissue and increasing their therapeutic effect [31]. On the other hand, we have previously found that pFUS may have an independent therapeutic effect in AKI, and can enhance MSC therapy independent of increased homing [33]. Consistent with our previous study, we have found here that pFUS is independently able to attenuate NLRP3-mediated inflammation, with subsequent improvements in physiological kidney function. Additionally, we demonstrate that pFUS acts synergistically with EV therapy to reverse AKI.

The NLRP3 inflammasome is an intracellular protein complex consisting of NLRP3, ASC, and pro-caspase-1, which upon activation releases active caspase-1 that proceeds to convert pro-inflammatory cytokines IL-1β and IL-18 into their mature form [42]. The inflammasome has been shown to be upregulated in both mouse models of AKI and human renal biopsies from different pathologies [6]. NLRP3 also has inflammasome-independent effects in tubular epithelial cells [9], including participating in SMAD2 and SMAD3 phosphorylation in response to TGF-β signaling, triggering renal fibrosis [43]. Though there have been previous reports on the suppression of the NLRP3 inflammasome by MSCs [44-46] and MSC-derived EVs [47,48], our study is the first to show that pFUS has both an independent and synergistic role in its regulation.

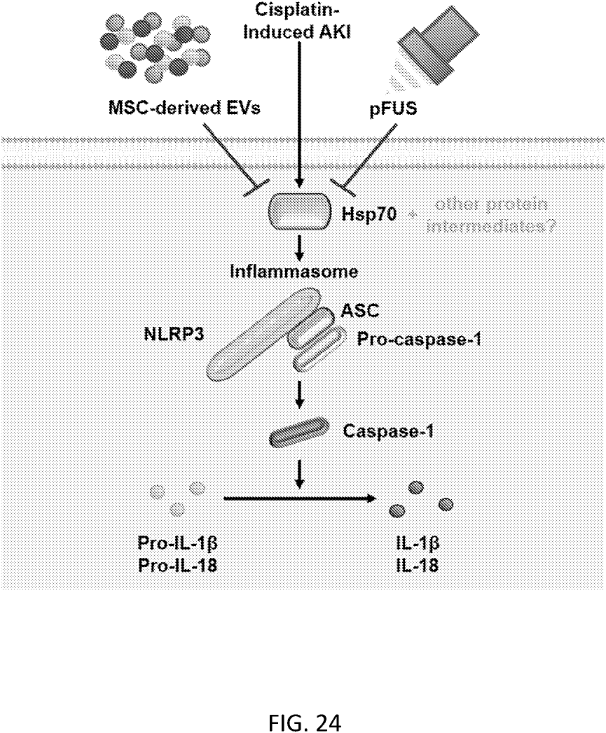

Heat shock proteins (HSPs) are molecular chaperones known to broadly regulate inflammation, including the formation of the NLRP3 inflammasome [34]. However, the exact direction of their HSP70, which in turn reduces the formation of the NLRP3 inflammasome, resulting in the attenuation of the pro-inflammatory environment characteristic of AKI (FIG. 24). regulation appears to be context dependent. HSP90 has been shown to be a positive regulator of the NLRP3 inflammasome in various studies [35-37]. HSP70 has also been shown to be a positive regulator of airway inflammation, with HSP70 knockout mice showing significant reductions in airway inflammation compared to wild type mice following intratracheal antigen challenge [39]. Extracellular HSP70 has been shown to act as a cytokine, binding to monocytes through CD14 and activating NF-kB signaling to increase the production of IL-1β, IL-6, and TNF-α [38]. On the contrary, intracellular HSP70 has also been shown to inhibit NLRP3 inflammasome activation in a mouse model of peritonitis [40], where HSP70 deficiency caused worsened NLRP3-dependent peritonitis and enhanced caspase-1 activation and IL-1 β production by macrophages, while genetic or heat shock-induced HSP70 overexpression had the opposite effect. The highly context-dependent effects of HSP70 on NLRP3 inflammasome regulation highlights the need for careful studies investigating their molecular links and the involved cell types, details that become crucial should HSP inhibitors be considered for preclinical investigation [49].

Our study has found that in cisplatin-induced AKI, HSP70, HSP90, and NLRP3 are all highly upregulated, and can be suppressed with pFUS and EV therapy. We found that HSP70 knockdown in vitro leads to significant suppression of NLRP3 expression, suggesting HSP70 to be a positive regulator of the NLRP3 inflammasome. We thus propose a mechanism by which pFUS and EVs, likely through intermediate effectors, converge to suppress HSP70, which reduces NLRP3 inflammasome formation and subsequent release of proinflammatory cytokines (FIG. 24). Alternative mechanisms must also be considered, including the possibility of another protein targeting both HSP70 and NLRP3. It would be necessary to repeat our experiments in an HSP70 knockout mouse before we can conclude whether the observed therapeutic effect is in fact dependent on HSP70.

In summary, our study demonstrates that pFUS has both independent and synergistic therapeutic effects when used in combination with MSC-derived EVs to treat cisplatin-induced AKI. Both pFUS and EV converge to suppress HSP70/90, which leads to decreased expression of the NLRP3 inflammasome and downstream pro-inflammatory cytokines, ultimately improving kidney function. The growing worldwide prevalence and morbidity of AKI prompts the development of regenerative therapies to restore kidney function and avoid progression to chronic kidney disease. Though EVs have seen substantial preclinical success for AKI, improving their therapeutic efficacy may be necessary for clinical translation. The safety profile and non-invasive nature of pFUS make it an attractive tool, though much remains to be understood about its physiological and molecular effects. Careful characterization of these mechanisms will serve to further its development and optimization as a clinical tool.

### 4. Methods

### 4.1. Animal Experiments

All experimental procedures were performed in accordance with guidelines from the Administrative Panel on Laboratory Animal Care (APLAC) of Stanford University. 40 female CD1 mice were purchased from Charles River Laboratories (Wilmington, MA, USA). Animals were 6 weeks old with body weight in the range of 25-30 g, and were housed for 1 week with 12 h light-dark cycles prior to the start of experiments.

CD1 mice were randomly divided into five groups with 8 animals in each group. Group 1 consisted of untreated control animals. Mice in groups 2-5 received a single intraperitoneal injection of cisplatin (10 mg/kg) on day 0 to induce AKI. Group 2: AKI control, which received no treatment; Group 3: AKI + EVs, which received EVs treatment at a dose of 200 µg/100 g body weight on day 3; Group 4: AKI + EVs + pFUS, which received pFUS on day 2 followed by EVs on day 3; Group 5: AKI + pFUS alone, which received pFUS on day 2. Mice were sacrificed at day 9 after cisplatin injection, at which point the blood and kidney samples were collected. Serum was obtained by centrifugation at 4° C. at 300_{×} g for 10 min and stored at -20° C. for further analysis. For histological analysis one kidney was immersed in 10% neutral buffered formalin, while the other kidney was frozen in liquid nitrogen for molecular analysis.

### 4.2. Extracellular Vesicle Isolation and Purification

Extracellular vesicles (EVs) were isolated from bone marrow-derived mesenchymal stromal cells (BM-MSCs) pooled from three human donors purchased from ATCC. BM-MSCs were cultured in modified Eagle’s medium (α-MEM) supplemented with 20% fetal bovine serum (FBS) and 100 U/mL penicillin and streptomycin (Thermo Fisher Scientific, Fremont, CA, USA), and incubated at 37° C. with 5% CO₂. Cells were maintained until passage 3, at which point cells were cultured for 5 more days until they reached 80-90% confluence. Cells were then incubated in fresh serum-free Dulbecco’s modified Eagle medium (DMEM) overnight. The resulting conditioned media was centrifuged at 5000× g for 10 min at 25° C. The supernatant from the previous step was then ultracentrifuged at 17,000× g for 20 min. The second supernatant was used to isolate EVs using an anion exchange resin (Q Sepharose Fast Flow, GE Healthcare, Chicago, IL, USA). The resin prepared in three steps: (1) balancing with 50 mM NaCl in 50 mM phosphate buffer, (2) washing with 100 mM NaCl in 50 mM phosphate buffer, and (3) rinsing with 500 mM NaCl in 50 mM phosphate buffer. The supernatant was then applied to the resin. EV fractions were collected, filter sterilized, and stored at 4° C. EVs were characterized by expression of surface markers (CD9, CD81, TSG101). EV size was characterized by nanoparticle tracking analysis (size range 20-180 nm, mean 118 nm, standard deviation 27 nm), as well as transmission electron microscopy (TEM), as previous reported [22].

### 4.3. Pulsed Focused Ultrasound

Pulsed focused ultrasound (pFUS) was conducted using a setup of co-aligned transducers with image guidance. pFUS was administered using a 1.1 MHz central frequency custom high-intensity focused ultrasound (HIFU) therapy transducer with 49 mM central opening (H-102NRE, Sonic Concepts, Bothell, WA, USA), with an imaging transducer (Siemens Acuson S2000 14L5 sp, Siemens Corporation, WA, USA) positioned at the central opening of the HIFU transducer. The HIFU transducer was calibrated in a water tank filled with degassed and deionized water as previously described [33]. A custom-made 3D-printed holder was used to align and fix both transducers in place, with the focal spot of the HIFU transducer secured at 55 mM axial and 0 mM lateral to the central point of the imaging transducer. Alignments of the HIFU and imaging beams were checked several times in a water tank containing a hydrophone and oscilloscope. All calibrations resulted in a beam misalignment of less than 200 µm. Mice were anesthetized and submerged vertically, with their heads kept above the water surface. The 3D-printer holder holding both HIFU and imaging transducers was then connected to a translation stage and placed in the water at about 50 mM axial distance from the mice. The imaging transducer was used to identify the mouse’s kidney, and the kidney was placed at the focal spot of the HIFU transducer 55 mM axially and 0 mM laterally from the central point of the imaging transducer. To treat the whole kidney, 8 non-overlapping adjacent regions through the kidney were targeted for 30 s per region. The time to treat one kidney with these parameters was approximately 4 min. In order to deliver pFUS therapy to the animal, the HIFU transducer was used with the following parameters: 5% duty cycle (DC), 5 Hz pulse repetition frequency (PRF), 2.9 MPa peak negative pressure (PNP), and 272 W/cm² spatial average pulse average intensity (_{ISAPA}). After pFUS treatment, each mouse was removed from the water bath, dried, and placed in a recovery cage.

### 4.4. Analysis of Kidney Function

ELISA kits were used to measure blood urea nitrogen (BUN) and serum creatinine (SCr) (Santa Cruz Biotechnology, Dallas, TX, USA), serum neutrophil gelatinase-associated lipocalin (NGAL) (R&D Systems, Minneapolis, MN, USA), and kidney injury molecule-1 (KIM-1), NLRP3, IL-6, and TNF-α (Cell Signaling Technology, Danvers, MA, USA). All samples were analyzed according to the manufacturer’s instructions.

### 4.5. Histology, Immunohistochemistry, and Immunofluorescence

Animals were perfused with 4% (vol/vol) paraformaldehyde in PBS, and whole kidney tissues were fixed in formalin for 24 h. Kidney tissues were then sectioned into 6 µm slices for hematoxylin-eosin and trichrome staining. For immunohistochemical staining, first the paraffin embedded sections were deparaffinized, hydrated and antigen-retrieved. Donkey serum was used to block the slides followed by incubation of slides at 4° C. overnight with primary antibodies against NLRP3, IL-1β and IL-18. Later the slides were incubated with secondary antibodies for 1 h and then incubated with 3,3-diaminobenzidine (DAB) (Vector Laboratories, Burlingame, CA, USA) and slides were viewed with a Nikon Eclipse 80i microscope prepared with a digital camera (Nikon, Melville, NY, USA).

### 4.6. NLRP3 Knockdown by siRNA and Overexpression by 7BIO

HEK cells were purchased from the American Type Culture Collection (ATCC, Manassas, VA, USA) and were grown until passage 3 in Dulbecco’s Modified Eagle’s Medium (DMEM) supplemented with 10% fetal bovine serum (FBS), 150 U/mL penicillin, and 150 mg/mL streptomycin. Passage 3 cells were transfected with NLRP3-specific siRNA (final concentration 25 nM; Thermo Fisher Scientific, Fremont, CA, USA) using lipofectamine RNAiMAX transfection reagent (Thermo Fisher Scientific, Fremont, CA, USA) according to the manufacturer’s instructions. Similarly, passage 3 HEK cells were incubated with 5 µM of 7-bromoindirubin-3'-oxime (7BIO) (Bioscience Visions, San Diego, CA, USA) for 24 h, to activate the NLRP3 inflammasome. Protein was isolated from above cells and quantified using Western blot as described below.

### 4.7. Western Blot Analysis

Thin sections of kidney tissue were sonicated and homogenized. The lysate was then placed in 1 × SDS sample buffer in association with radioimmunoprecipitation buffer (RIPA) solution comprising 1% NP40, 0.1% SDS, 100 mg/mL PMSF, 1% protease inhibitor cocktail, and 1% phosphatase I and II inhibitor cocktail (Sigma Aldrich, St Louis, MO, USA) on ice. The supernatant was collected by centrifugation at 13,000× g at 4° C. for 30 min. Protein concentration was measured using a bicinchoninic acid protein assay. An equal amount of protein was then loaded into 10% or 15% SDS-PAGE and transferred onto polyvinylidene difluoride membranes. The primary antibodies were as follows: HSP70 (sc-32239, Santa Cruz Biotechnology, Dallas, TX, USA, 1:400 dilution), HSP90 (sc-101494, Santa Cruz Biotechnology, Dallas, TX, USA, 1:400 dilution), NLRP3 (sc06-23, Invitrogen, Waltham, MA, USA, 1:400dilution), IL-1β (M421B, Thermo Fisher Scientific, Fremont, CA, USA, 1:200 dilution), IL-18 (PA5-79481, Thermo Fisher Scientific, Fremont, CA, USA, 1:200 dilution), anti-β-actin (sc-1616, Santa Cruz Biotechnology, Dallas, TX, USA, 1:200 dilution). Quantification of the Western blot was done by measuring the intensity of the signals using National Institutes of Health Image software package and Bio-Rad image software (Bio-Rad, Hercules, CA, USA).

### 4.8. Quantitative Polymerase Chain Reaction

Extraction of RNA from homogenized kidney tissues was performed using Triazole Reagent (Sigma Aldrich, St Louis, MO, USA) and digested using DNase 1. Reverse transcription was done using reverse transcriptase kit per manufacturer’s instructions (Applied Biosystems, Fremont, CA, USA). cDNA quality was measured by calculating the ratio of absorbance at 260 and 280 nm using an Agilent 2100 Bioanalyzer (Agilent, Santa Clara, CA, USA). Amplification of cDNA was performed using an iCycler Thermal Cycler (Bio-Rad, Hercules, CA, USA) with SYBR Green (Applied Biosystems, Fremont, CA, USA) and specific primers for NLRP3 (Mm00840904-m1), ASC (Mm00445747-g1) and Caspase-1 (Mm00438023-m1), and GAPDH (Mm99999915-g1) which was used as a housekeeping gene (Thermo Fisher, Fremont, CA, USA). Expression of these genes was normalized to GAPDH expression and calculated using the formula 2-^{ΔΔCt} and expressed in % GAPDH expression.

### 4.9. Statistical Analysis

All data are presented as the mean ± standard deviation (SD) derived from at least three separate independent experiments. Three or more samples were analyzed with analysis of variance (ANOVA) followed by Tukey’s multiple comparison test. Statistical analysis was completed using the GraphPad Prism 6.0.4 software (GraphPad Software, San Diego, CA, USA).

### References

1. Levey, A.S.; James, M.T. Acute Kidney Injury. Ann. Intern. Med. 2017, 167, ITC66-ITC80.

2. Susantitaphong, P.; Cruz, D.N.; Cerdá, J.; Abulfaraj, M.; Alqahtani, F.; Koulouridis, I.; Jaber, B.L. World incidence of AKI: A meta-analysis. Clin. J. Am. Soc. Nephrol. 2013, 8, 1482-1493.

3. Rabb, H.; Griffin, M.D.; McKay, D.B.; Swaminathan, S.; Pickkers, P.; Rosner, M.H.; Kellum, J.A.; Ronco, C. Inflammation in AKI: Current Understanding, Key Questions, and Knowledge Gaps. J. Am. Soc. Nephrol. 2016, 27, 371-379.

4. Medzhitov, R. Inflammation 2010: New adventures of an old flame. Cell 2010, 140, 771-776.

5. Kurts, C.; Panzer, U.; Anders, H.J.; Rees, A.J. The immune system and kidney disease: Basic concepts and clinical implications. Nat. Rev. Immunol. 2013, 13, 738-753.

6. Vilaysane, A.; Chun, J.; Seamone, M.E.; Wang, W.; Chin, R.; Hirota, S.; Li, Y.; Clark, S.A.; Tschopp, J.; Trpkov, K.; et al. The NLRP3 inflammasome promotes renal inflammation and contributes to CKD. J. Am. Soc. Nephrol. 2010, 21, 1732-1744.

7. Valles, P.G.; Lorenzo, A.G.; Bocanegra, V.; Valles, R. Acute kidney injury: What part do toll-like receptors play? Int. J. Nephrol. Renov. Dis. 2014, 7, 241-251 .

8. Bolisetty, S.; Agarwal, A. Neutrophils in acute kidney injury: Not neutral any more. Kidney Int. 2009, 75, 674-676.

9. Shigeoka, A.A.; Mueller, J.L.; Kambo, A.; Mathison, J.C.; King, A.J.; Hall, W.F.; Correia Jda, S.; Ulevitch, R.J.; Hoffman, H.M.; McKay, D.B. An inflammasome-independent role for epithelial-expressed Nlrp3 in renal ischemia-reperfusion injury. J. Immunol. 2010, 185, 6277-6285.

10. Shigeoka, A.A.; Kambo, A.; Mathison, J.C.; King, A.J.; Hall, W.F.; da Silva Correia, J.; Ulevitch, R.J.; McKay, D.B. Nod1 and nod2 are expressed in human and murine renal tubular epithelial cells and participate in renal ischemia reperfusion injury. J. Immunol. 2010, 184, 2297-2304.

11. Shigeoka, A.A.; Holscher, T.D.; King, A.J.; Hall, F.W.; Kiosses, W.B.; Tobias, P.S.; Mackman, N.; McKay, D.B. TLR2 is constitutively expressed within the kidney and participates in ischemic renal injury through both MyD88-dependent and -independent pathways. J. Immunol. 2007, 178, 6252-6258.

12. Kim, M.G.; Koo, T.Y.; Yan, J.J.; Lee, E.; Han, K.H.; Jeong, J.C.; Ro, H.; Kim, B.S.; Jo, S.K.; Oh, K.H.; et al. IL-2/anti-IL-2 complex attenuates renal ischemia-reperfusion injury through expansion of regulatory T cells. J. Am. Soc. Nephrol. 2013, 24, 1529-1536.

13. Wang, M.; Yuan, Q.; Xie, L. Mesenchymal Stem Cell-Based Immunomodulation: Properties and Clinical Application. Stem. Cells Int. 2018, 2018, 3057624.

14. Herrera, M.B.; Bussolati, B.; Bruno, S.; Fonsato, V.; Romanazzi, G.M.; Camussi, G. Mesenchymal stem cells contribute to the renal repair of acute tubular epithelial injury. Int. J. Mol. Med. 2004, 14, 1035-1041.

15. Morigi, M.; Imberti, B.; Zoja, C.; Corna, D.; Tomasoni, S.; Abbate, M.; Rottoli, D.; Angioletti, S.; Benigni, A.; Perico, N.; et al. Mesenchymal stem cells are renotropic, helping to repair the kidney and improve function in acute renal failure. J. Am. Soc. Nephrol. 2004, 15, 1794-1804.

16. Morigi, M.; De Coppi, P. Cell therapy for kidney injury: Different options and mechanisms-Mesenchymal and amniotic fluid stem cells. Nephron Exp. Nephrol. 2014, 126, 59.

17. Lai, R.C.; Yeo, R.W.; Lim, S.K. Mesenchymal stem cell exosomes. Semin Cell Dev. Biol. 2015, 40, 82-88.

18. Ullah, M.; Liu, D.D.; Thakor, A.S. Mesenchymal Stromal Cell Homing: Mechanisms and Strategies for Improvement. iScience 2019, 15, 421-438.

19. Schubert, R.; Sann, J.; Frueh, J.T.; Ullrich, E.; Geiger, H.; Baer, P.C. Tracking of Adipose-Derived Mesenchymal Stromal/Stem Cells in a Model of Cisplatin-Induced Acute Kidney Injury: Comparison of Bioluminescence Imaging versus qRT-PCR. Int. J. Mol. Sci. 2018, 19, 2564.

20. Lv, L.L.; Wu, W.J.; Feng, Y.; Li, Z.L.; Tang, T.T.; Liu, B.C. Therapeutic application of extracellular vesicles in kidney disease: Promises and challenges. J. Cell Mol. Med. 2018, 22, 728-737.

21. Phinney, D.G.; Pittenger, M.F. Concise Review: MSC-Derived Exosomes for Cell-Free Therapy. Stem Cells 2017, 35, 851-858.

22. Ullah, M.; Liu, D.D.; Rai, S.; Razavi, M.; Choi, J.; Wang, J.; Concepcion, W.; Thakor, A.S. A Novel Approach to Deliver Therapeutic Extracellular Vesicles Directly into the Mouse Kidney via Its Arterial Blood Supply. Cells 2020, 9, 937.

23. Schrepfer, S.; Deuse, T.; Reichenspurner, H.; Fischbein, M.P.; Robbins, R.C.; Pelletier, M.P. Stem cell transplantation: The lung barrier. Transplant. Proc. 2007, 39, 573-576.

24. Santeramo, I.; Herrera Perez, Z.; Illera, A.; Taylor, A.; Kenny, S.; Murray, P.; Wilm, B.; Gretz, N. Human kidney-derived cells ameliorate acute kidney injury without engrafting into renal tissue. Stem Cells Transl. Med. 2017, 6, 1373-1384.

25. Leibacher, J.; Henschler, R. Biodistribution, migration and homing of systemically applied mesenchymal stem/stromal cells. Stem Cell Res. Ther. 2016, 7, 7.

26. Eggenhofer, E.; Benseler, V.; Kroemer, A.; Popp, F.C.; Geissler, E.K.; Schlitt, H.J.; Baan, C.C.; Dahlke, M.H.; Hoogduijn, M.J. Mesenchymal stem cells are short-lived and do not migrate beyond the lungs after intravenous infusion. Front. Immunol. 2012, 3, 297.

27. Fischer, U.M.; Harting, M.T.; Jimenez, F.; Monzon-Posadas, W.O.; Xue, H.; Savitz, S.I.; Laine, G.A.; Cox, C.S., Jr. Pulmonary passage is a major obstacle for intravenous stem cell delivery: The pulmonary first-pass effect. Stem Cells Dev. 2009, 18, 683-692.

28. Lou, G.; Chen, Z.; Zheng, M.; Liu, Y. Mesenchymal stem cell-derived exosomes as a new therapeutic strategy for liver diseases. Exp. Mol. Med. 2017, 49, e346.

29. Liu, D.D.; Ullah, M.; Concepcion, W.; Dahl, J.J.; Thakor, A.S. The role of ultrasound in enhancing mesenchymal stromal cell-based therapies. Stem Cells Transl. Med. 2020.

30. Ziadloo, A.; Burks, S.R.; Gold, E.M.; Lewis, B.K.; Chaudhry, A.; Merino, M.J.; Frenkel, V.; Frank, J.A. Enhanced homing permeability and retention of bone marrow stromal cells by noninvasive pulsed focused ultrasound. Stem Cells 2012, 30, 1216-1227.

31. Burks, S.R.; Nguyen, B.A.; Tebebi, P.A.; Kim, S.J.; Bresler, M.N.; Ziadloo, A.; Street, J.M.; Yuen, P.S.; Star, R.A.; Frank, J.A. Pulsed focused ultrasound pretreatment improves mesenchymal stromal cell efficacy in preventing and rescuing established acute kidney injury in mice. Stem Cells 2015, 33, 1241-1253.

32. Burks, S.R.; Nagle, M.E.; Bresler, M.N.; Kim, S.J.; Star, R.A.; Frank, J.A. Mesenchymal stromal cell potency to treat acute kidney injury increased by ultrasound-activated interferon-gamma/interleukin-10 axis. J. Cell Mol. Med. 2018, 22, 6015-6025.

33. Ullah, M.; Liu, D.D.; Rai, S.; Dadhania, A.; Jonnakuti, S.; Concepcion, W.; Thakor, A.S. Reversing Acute Kidney Injury Using Pulsed Focused Ultrasound and MSC Therapy: A Role for HSP-Mediated PI3K/AKT Signaling. Mol. Ther. Methods Clin. Dev. 2020, 17, 683-694.

34. Martine, P.; Rebe, C. Heat Shock Proteins and Inflammasomes. Int. J. Mol. Sci. 2019, 20, 4508.

35. Mayor, A.; Martinon, F.; De Smedt, T.; Petrilli, V.; Tschopp, J. A crucial function of SGT1 and HSP90 in inflammasome activity links mammalian and plant innate immune responses. Nat. Immunol. 2007, 8, 497-503.

36. Piippo, N.; Korhonen, E.; Hytti, M.; Skottman, H.; Kinnunen, K.; Josifovska, N.; Petrovski, G.; Kaarniranta, K.; Kauppinen, A. Hsp90 inhibition as a means to inhibit activation of the NLRP3 inflammasome. Sci. Rep. 2018, 8, 6720.

37. Zuo, Y.; Wang, J.; Liao, F.; Yan, X.; Li, J.; Huang, L.; Liu, F. Inhibition of Heat Shock Protein 90 by 17-AAG Reduces Inflammation via P2X7 Receptor/NLRP3 Inflammasome Pathway and Increases Neurogenesis After Subarachnoid Hemorrhage in Mice. Front. Mol. Neurosci. 2018, 11, 401.

38. Asea, A.; Kraeft, S.K.; Kurt-Jones, E.A.; Stevenson, M.A.; Chen, L.B.; Finberg, R.W.; Koo, G.C.; Calderwood, S.K. HSP70 stimulates cytokine production through a CD14-dependant pathway, demonstrating its dual role as a chaperone and cytokine. Nat. Med. 2000, 6, 435-442.

39. Yombo, D.J.K.; Mentink-Kane, M.M.; Wilson, M.S.; Wynn, T.A.; Madala, S.K. Heat shock protein 70 is a positive regulator of airway inflammation and goblet cell hyperplasia in a mouse model of allergic airway inflammation. J. Biol. Chem. 2019, 294, 15082-15094.

40. Martine, P.; Chevriaux, A.; Derangere, V.; Apetoh, L.; Garrido, C.; Ghiringhelli, F.; Rebe, C. HSP70 is a negative regulator of NLRP3 inflammasome activation. Cell Death Dis. 2019, 10, 256.

41. Tempany, C.M.; McDannold, N.J.; Hynynen, K.; Jolesz, F.A. Focused ultrasound surgery in oncology: Overview and principles. Radiology 2011, 259, 39-56.

42. Guo, H.; Callaway, J.B.; Ting, J.P. Inflammasomes: Mechanism of action, role in disease, and therapeutics. Nat. Med. 2015, 21, 677-687.

43. Wang, W.; Wang, X.; Chun, J.; Vilaysane, A.; Clark, S.; French, G.; Bracey, N.A.; Trpkov, K.; Bonni, S.; Duff, H.J.; et al. Inflammasome-independent NLRP3 augments TGF-beta signaling in kidney epithelium. J. Immunol. 2013, 190, 1239-1249.

44. Oh, J.Y.; Ko, J.H.; Lee, H.J.; Yu, J.M.; Choi, H.; Kim, M.K.; Wee, W.R.; Prockop, D.J. Mesenchymal stem/stromal cells inhibit the NLRP3 inflammasome by decreasing mitochondrial reactive oxygen species. Stem Cells 2014, 32, 1553-1563.

45. Miteva, K.; Pappritz, K.; Sosnowski, M.; El-Shafeey, M.; Muller, I.; Dong, F.; Savvatis, K.; Ringe, J.; Tschope, C.; Van Linthout, S. Mesenchymal stromal cells inhibit NLRP3 inflammasome activation in a model of Coxsackievirus B3-induced inflammatory cardiomyopathy. Sci. Rep. 2018, 8, 2820.

46. Li, S.; Wu, H.; Han, D.; Ma, S.; Fan, W.; Wang, Y.; Zhang, R.; Fan, M.; Huang, Y.; Fu, X.; et al. A Novel Mechanism of Mesenchymal Stromal Cell-Mediated Protection against Sepsis: Restricting Inflammasome Activation in Macrophages by Increasing Mitophagy and Decreasing Mitochondrial ROS. Oxidative Med. Cell Longev. 2018, 2018, 3537609.

47. Liu, Y.; Lou, G.; Li, A.; Zhang, T.; Qi, J.; Ye, D.; Zheng, M.; Chen, Z. AMSC-derived exosomes alleviate lipopolysaccharide/d-galactosamine-induced acute liver failure by miR-17-mediated reduction of TXNIP/NLRP3 inflammasome activation in macrophages. EBioMedicine 2018, 36, 140-150.

48. Chen, L.; Lu, F.B.; Chen, D.Z.; Wu, J.L.; Hu, E.D.; Xu, L.M.; Zheng, M.H.; Li, H.; Huang, Y.; Jin, X.Y.; et al. BMSCs-derived miR-223-containing exosomes contribute to liver protection in experimental autoimmune hepatitis. Mol. Immunol. 2018, 93, 38-46.

49. Chatterjee, S.; Burns, T.F. Targeting Heat Shock Proteins in Cancer: A Promising Therapeutic Approach. Int. J. Mol. Sci. 2017, 18, 1978.

1. A method of treating damaged kidney tissue in a subject, the method comprising locally administering to the damaged kidney tissue a therapeutically effective amount of pulsed focused ultrasound (pFUS) therapy in combination with a therapeutically effective amount of mesenchymal stromal cells (MSCs) or MSC-derived extracellular vesicles.

2. The method of claim 1, wherein the MSCs or the MSC-derived extracellular vesicles are administered locally to the damaged kidney tissue intra-arterially via a renal artery.

3. The method of claim 1, wherein the pFUS therapy is administered with an ultrasound frequency ranging from 20 kHz to 5.0 MHz, a pulse repetition frequency (PRF) of about 5 Hz, an ultrasound duty cycle ranging from 0.01% to 100%, and a negative peak pressure (NPP) ranging from 0.1 MPa to 10 MPa.

4-12. (canceled)

13. The method of claim 1, wherein the pFUS therapy is administered to the subject for at least 30 seconds.

14. The method of claim 13, wherein the pFUS therapy is administered to the subject for a period ranging from about 1 minute to about 5 minutes.

15. The method of claim 1, wherein the pFUS therapy is administered at a single location or at multiple locations in the kidney.

16. The method of claim 1, further comprising imaging the damaged kidney tissue.

17. The method of claim 16, wherein said imaging is performed by ultrasound, magnetic resonance imaging (MRI), computed tomography (CT), or scintigraphy.

18. The method of claim 1, wherein the MSCs are from bone marrow or adipose tissue.

19. The method of claim 1, wherein the kidney tissue is damaged from an acute kidney injury or chronic kidney disease.

20. The method of claim 19, wherein the acute kidney injury is caused by chemotherapy, a chemical exposure, surgery, or a traumatic physical injury.

21. The method of claim 1, wherein the MSC-derived extracellular vesicles are exosomes or microvesicles.

22. The method of claim 21, wherein the MSC-derived exosomes comprise one or more surface markers selected from the group consisting of CD9, CD63, and TSG101.

23. (canceled)

24. The method of claim 1, wherein the MSC-derived extracellular vesicles have diameters ranging from about 20 nm to 180 nm.

25. The method of claim 24, wherein the MSC-derived extracellular vesicles have a mean diameter of 118 nm.

26. The method of claim 1, wherein multiple cycles of treatment are administered to the subject.

27. A method of suppressing NLRP3 inflammasome-mediated inflammation in a subject, the method comprising locally administering to damaged kidney tissue an effective amount of pulsed focused ultrasound (pFUS) in combination with an effective amount of mesenchymal stromal cells (MSCs) or MSC-derived extracellular vesicles.

28. The method of claim 27, wherein the MSCs or the MSC-derived extracellular vesicles are administered locally to the damaged kidney tissue intra-arterially via a renal artery.

29. The method of claim 27, wherein the pFUS is administered with an ultrasound frequency ranging from 20 kHz to 5.0 MHz, a pulse repetition frequency (PRF) of about 5 Hz, an ultrasound duty cycle ranging from 0.01% to 100%, and a negative peak pressure (NPP) ranging from 0.1 MPa to 10 MPa.

30-40. (canceled)

41. The method of claim 27, wherein the pFUS therapy is administered at a single location or at multiple locations in the kidney.

42-49. (canceled)
